SEQUENCE LISTING (378 sequences)

SEQ ID NO: 1 - DNA, 23 bp, Enterococcus faecalis
acaggtgttgaaatgttccgtaa

SEQ ID NO: 2 - DNA, 23 bp, Enterococcus faecalis
acgtctgttgtacggaagtagaa

SEQ ID NO: 3 - DNA, 23 bp, Clostridium perfringens
caggaatcgaaatgttcagaaag

SEQ ID NO: 4 - DNA, 23 bp, Clostridium perfringens
acgtctgttgttctgaagtagaa

SEQ ID NO: 5 - DNA, 23 bp, Corynebacterium jeikeium
acctccatcgagatgttcaacaa

SEQ ID NO: 6 - DNA, 18 bp, Corynebacterium jeikeium
ggtggtgcggaagtagaa

SEQ ID NO: 7 - DNA, 23 bp, Capnocytophaga canimorsus
acaggagttgagatgttccgtaa

SEQ ID NO: 8 - DNA, 23 bp, Capnocytophaga canimorsus
acgtcagttgtacgaacatagaa

SEQ ID NO: 9 - DNA, 21 bp, Artificial Sequence
  primer that targets broad spectrum of bacterial species
ggtwgtngctgcgactgacgg

SEQ ID NO: 10 - DNA, 20 bp, Artifical Sequence
  primer that targets broad spectrum of bacterial species
tcaatcgcacgctctggttc

SEQ ID NO: 11 - DNA, 19 bp, Artificial Sequence
  Staphylococcus sp.
aacgtggtcaagtwttagc

SEQ ID NO: 12 - DNA, 20 bp, Artificial Sequence
  Staphylococcus sp.
gtacggaartagaattgwgg

SEQ ID NO: 13 - DNA, 21 bp, Artificial Sequence
  Streptococcus sp.
gtggratngcngcctttatcg

SEQ ID NO: 14 - DNA, 20 bp, Artificial Sequence
  Streptococcus sp.
atngcctgrctcatcatacg

SEQ ID NO: 15 - DNA, 24 bp, Artificial Sequence
  Candida sp.
caagatggaytcygtyaantggga

SEQ ID NO: 16 - DNA, 24 bp, Artificial Sequence
  Candida sp.
catcttgcaatggcaatctcaatg

SEQ ID NO: 17 - DNA, 24 bp, Candida krusei
catcttgtaatggtaatcttaatg

SEQ ID NO: 18 - DNA, 22 bp, Artificial Sequence
  Aspergillus sp.
gttccagacynccaagtatgag

SEQ ID NO: 19 - DNA, 23 bp, Artificial Sequence
  Aspergillus sp.
atttcgttgtaacgatcctcgga

SEQ ID NO: 20 - DNA, 24 bp, Aspergillus flavus
gatttcgttgtaacgatcctgaga

SEQ ID NO: 21 - DNA, 23 bp, Aspergillus terreus
atttcgttgtaacggtcctcaga

SEQ ID NO: 22 - DNA, 20 bp, Artificial Sequence
  primer that targets broad spectrum of bacterial species
tgatgccgrtngaagacgtg

SEQ ID NO: 23 - DNA, 20 bp, Artificial Sequence
  primer that targets broad spectrum of bacterial species
agyttgcggaacatttcaac

SEQ ID NO: 24 - DNA, 16 bp, Streptomyces avermitilis
ggccagtccgtcctcg

SEQ ID NO: 25 - DNA, 18 bp, Streptomyces avermitilis
gatgccggtgaccgtggt

SEQ ID NO: 26 - DNA, 19 bp, Artificial Sequence
  Escherichia coli / Shigella sp.
gtgggaagcgaaaatcctg

SEQ ID NO: 27 - DNA, 20 bp, Acinetobacter baumannii
tacttctgcgtcgaatttag

SEQ ID NO: 28 - DNA, 18 bp, Acinetobacter baumannii
acttctgcgtcgaattta

SEQ ID NO: 29 - DNA, 17 bp, Acinetobacter baumannii
cttctgcgtcgaattta

SEQ ID NO: 30 - DNA, 20 bp, Acinetobacter baumannii
gtaaccatttaagaatggag

SEQ ID NO: 31 - DNA, 18 bp, Acinetobacter baumannii
aaccatttaagaatggag

SEQ ID NO: 32 - DNA, 20 bp, Acinetobacter lwoffii
cacgaagaagaacaccacag

SEQ ID NO: 33 - DNA, 17 bp, Acinetobacter lwoffii
gaagaagaacaccacag

SEQ ID NO: 34 - DNA, 20 bp, Aeromonas caviae
ttcacgcttcacgccacgca

SEQ ID NO: 35 - DNA, 18 bp, Aeromonas caviae
tcacgcttcacgccacgc

SEQ ID NO: 36 - DNA, 18 bp, Aeromonas caviae
cggtagcccttgaagaac

SEQ ID NO: 37 - DNA, 17 bp, Aeromonas caviae
ggtagcccttgaagaac

SEQ ID NO: 38 - DNA, 20 bp, Aeromonas hydrophila
cagtgcaccgatgttctcgc

SEQ ID NO: 39 - DNA, 20 bp, Aeromonas hydrophila
acgcagcagtgcaccgatgt

SEQ ID NO: 40 - DNA, 18 bp, Aeromonas hydrophila
acgcagcagtgcaccgat

SEQ ID NO: 41 - DNA, 20 bp, Aeromonas hydrophila
gaagaacggggtatgacgac

SEQ ID NO: 42 - DNA, 18 bp, Aeromonas hydrophila
agaacggggtatgacgac

SEQ ID NO: 43 - DNA, 17 bp, Aeromonas hydrophila
gaacggggtatgacgac

SEQ ID NO: 44 - DNA, 20 bp, Artificial Sequence
  Bacillus anthracis / Bacillus cereus
acagaaccgctttttgcaag

SEQ ID NO: 45 - DNA, 20 bp, Artificial Sequence
  Bacillus anthracis / Bacillus cereus
tgaatttagcgtgagctttt

SEQ ID NO: 46 - DNA, 20 bp, Artificial Sequence
  Bacillus anthracis / Bacillus cereus
agataatacgaaaacttcag

SEQ ID NO: 47 - DNA, 18 bp, Artificial Sequence
  Bacillus anthracis / Bacillus cereus
agataatacgaaaacttc

SEQ ID NO: 48 - DNA, 20 bp, Bacillus subtilis
ttgaatttgctgtgtggagt

SEQ ID NO: 49 - DNA, 18 bp, Bacillus subtilis
tgaatttgctgtgtggag

SEQ ID NO: 50 - DNA, 20 bp, Capnocytophaga canimorsus
tgcttcaccacggtcaagga

SEQ ID NO: 51 - DNA, 18 bp, Capnocytophaga canimorsus
cttcaccacggtcaagga

SEQ ID NO: 52 - DNA, 20 bp, Capnocytophaga canimorsus
ttgatttcagttttatcgat

SEQ ID NO: 53 - DNA, 20 bp, Citrobacter braakii
ttcttcacgcttgataccac

SEQ ID NO: 54 - DNA, 18 bp, Citrobacter braakii
ttcttcacgcttgatacc

SEQ ID NO: 55 - DNA, 17 bp, Citrobacter braakii
ttcttcacgcttgatac

SEQ ID NO: 56 - DNA, 20 bp, Artificial Sequence
  Citrobacter braakii / Klebsiella oxytoca
cggcttgatagagcccggct

SEQ ID NO: 57 - DNA, 18 bp, Artificial Sequence
  Citrobacter braakii / Klebsiella oxytoca
cggcttgatagagcccgg

SEQ ID NO: 58 - DNA, 17 bp, Artificial Sequence
  Citrobacter braakii / Klebsiella oxytoca
cggcttgatagagcccg

SEQ ID NO: 59 - DNA, 16 bp, Artificial Sequence
  Citrobacter braakii / Klebsiella oxytoca
cggcttgatagagccc

SEQ ID NO: 60 - DNA, 18 bp, Citrobacter freundii complexe
cccggcttagccagtacc

SEQ ID NO: 61 - DNA, 20 bp, Clostridium perfringens
attgttccaacttgagctaa

SEQ ID NO: 62 - DNA, 18 bp, Clostridium perfringens
attgttccaacttgagct

SEQ ID NO: 63 - DNA, 20 bp, Corynebacterium jeikeium
tgcggggtgtactcgcccgg

SEQ ID NO: 64 - DNA, 18 bp, Corynebacterium jeikeium
tgcggggtgtactcgccc

SEQ ID NO: 65 - DNA, 17 bp, Corynebacterium jeikeium
tgcggggtgtactcgcc

SEQ ID NO: 66 - DNA, 16 bp, Corynebacterium jeikeium
tgcggggtgtactcgc

SEQ ID NO: 67 - DNA, 20 bp, Enterobacter aerogenes
ggcttgatgctgcccggctt

SEQ ID NO: 68 - DNA, 18 bp, Enterobacter aerogenes
ggcttgatgctgcccggc

SEQ ID NO: 69 - DNA, 18 bp, Enterobacter cloacae complex
gcctggcttcgccagaac

SEQ ID NO: 70 - DNA, 18 bp, Enterobacter cloacae complex
ggcttgattgagcctggc

SEQ ID NO: 71 - DNA, 17 bp, Enterobacter cloacae complex
ggcttgattgagcctgg

SEQ ID NO: 72 - DNA, 20 bp, Enterobacter sakazakii
gttctcgcccgcacggcctt

SEQ ID NO: 73 - DNA, 18 bp, Enterobacter sakazakii
tctcgcccgcacggcctt

SEQ ID NO: 74 - DNA, 17 bp, Enterobacter sakazakii
tctcgcccgcacggcct

SEQ ID NO: 75 - DNA, 16 bp, Enterobacter sakazakii
ttctcgcccgcacggc

SEQ ID NO: 76 - DNA, 19 bp, Enterobacter sakazakii
cacctacgttctcgcccgc

SEQ ID NO: 77 - DNA, 17 bp, Enterobacter sakazakii
cctacgttctcgcccgc

SEQ ID NO: 78 - DNA, 20 bp, Enterococcus faecalis
gtgattgtagctggtttagc

SEQ ID NO: 79 - DNA, 18 bp, Enterococcus faecalis
gtgattgtagctggttta

SEQ ID NO: 80 - DNA, 19 bp, Enterococcus faecalis
ttttgtgtgtggagtgatt

SEQ ID NO: 81 - DNA, 20 bp, Enterococcus faecalis
tacttcagctttgaattttg

SEQ ID NO: 82 - DNA, 19 bp, Enterococcus faecium
gagcgtagtctaacaattt

SEQ ID NO: 83 - DNA, 18 bp, Enterococcus faecium
agcgtagtctaacaattt

SEQ ID NO: 84 - DNA, 20 bp, Artificial Sequence
  Enterococcus faecium / Enterococcus hirae
gtgtgattgtacctggttta

SEQ ID NO: 85 - DNA, 17 bp, Artificial Sequence
  Enterococcus faecium / Enterococcus hirae
tgtgattgtacctggtt

SEQ ID NO: 86 - DNA, 20 bp, Artificial Sequence
  Enterococcus faecium / Enterococcus hirae
ttcttcttttgtcaacacgt

SEQ ID NO: 87 - DNA, 17 bp, Artificial Sequence
  Enterococcus faecium / Enterococcus hirae
cttcttttgtcaacacg

SEQ ID NO: 88 - DNA, 20 bp, Artificial Sequence
  Escherichia coli / Escherichia fergusonii / Shigella sp. / Salmonella choleraesuis
gcttgatggtgcccggctta

SEQ ID NO: 89 - DNA, 18 bp, Artificial Sequence
  Escherichia coli / Escherichia fergusonii / Shigella sp. / Salmonella choleraesuis
cttgatggtgcccggctt

SEQ ID NO: 90 - DNA, 20 bp, Gemella haemolysans
acgttcgatgtcttcacgag

SEQ ID NO: 91 - DNA, 18 bp, Gemella haemolysans
gttcgatgtcttcacgag

SEQ ID NO: 92 - DNA, 17 bp, Gemella haemolysans
ttcgatgtcttcacgag

SEQ ID NO: 93 - DNA, 20 bp, Gemella morbillorum
acatcagctacgaattgagt

SEQ ID NO: 94 - DNA, 18 bp, Gemella morbillorum
catcagctacgaattgag

SEQ ID NO: 95 - DNA, 17 bp, Gemella morbillorum
atcagctacgaattgag

SEQ ID NO: 96 - DNA, 20 bp, Haemophilus influenzae
accgatgttttcacctgcac

SEQ ID NO: 97 - DNA, 17 bp, Haemophilus influenzae
cgatgttttcacctgca

SEQ ID NO: 98 - DNA, 16 bp, Haemophilus influenzae
cgatgttttcacctgc

SEQ ID NO: 99 - DNA, 20 bp, Haemophilus influenzae
ttgaacctggtttcgctaat

SEQ ID NO: 100 - DNA, 18 bp, Haemophilus influenzae
tgaacctggtttcgctaa

SEQ ID NO: 101 - DNA, 20 bp, Kingella kingae
cacgcaacaatacaccaacg

SEQ ID NO: 102 - DNA, 20 bp, Kingella kingae
cacgcaataatacaccaacg

SEQ ID NO: 103 - DNA, 20 bp, Kingella kingae
cttcagcttcaaatttagtg

SEQ ID NO: 104 - DNA, 20 bp, Kingella kingae
ttcttctttgctcaacacat

SEQ ID NO: 105 - DNA, 20 bp, Kingella kingae
ttcttctttgctcaatacat

SEQ ID NO: 106 - DNA, 18 bp, Klebsiella oxytoca
tgcggcttgatagagccc

SEQ ID NO: 107 - DNA, 20 bp, Klebsiella oxytoca
ttggacaggatataaacttc

SEQ ID NO: 108 - DNA, 20 bp, Klebsiella oxytoca
agtgtgacggccgccttcgt

SEQ ID NO: 109 - DNA, 20 bp, Klebsiella pneumoniae
cgggttgatggtgcccggct

SEQ ID NO: 110 - DNA, 18 bp, Klebsiella pneumoniae
gggttgatggtgcccggc

SEQ ID NO: 111 - DNA, 17 bp, Klebsiella pneumoniae
ggttgatggtgcccggc

SEQ ID NO: 112 - DNA, 16 bp, Klebsiella pneumoniae
gttgatggtgcccggc

SEQ ID NO: 113 - DNA, 20 bp, Morganella morganii
cagaacaccgacgttctcac

SEQ ID NO: 114 - DNA, 17 bp, Morganella morganii
gaacaccgacgttctca

SEQ ID NO: 115 - DNA, 20 bp, Morganella morganii
ttcgatttcttcacgcttgg

SEQ ID NO: 116 - DNA, 18 bp, Morganella morganii
cgatttcttcacgcttgg

SEQ ID NO: 117 - DNA, 17 bp, Morganella morganii
gatttcttcacgcttgg

SEQ ID NO: 118 - DNA, 20 bp, Neisseria gonorrhoeae
gttggcgaaaaacggggtat

SEQ ID NO: 119 - DNA, 18 bp, Neisseria gonorrhoeae
ttggcgaaaaacggggta

SEQ ID NO: 120 - DNA, 20 bp, Neisseria meningitidis
tcttctttgctcagtacgta

SEQ ID NO: 121 - DNA, 18 bp, Neisseria meningitidis
cttctttgctcagtacgt

SEQ ID NO: 122 - DNA, 20 bp, Neisseria meningitidis
cggtagttggcgaagaacgg

SEQ ID NO: 123 - DNA, 18 bp, Neisseria meningitidis
cggtagttggcgaagaac

SEQ ID NO: 124 - DNA, 17 bp, Neisseria meningitidis
ggtagttggcgaagaac

SEQ ID NO: 125 - DNA, 20 bp, Pasteurella multocida
ttttgataacacgtaaactt

SEQ ID NO: 126 - DNA, 40 bp, Artificial Sequence
  Internal control tag sequence derivated from Pseudomonas aeruginosa
ctggtcggcataggacggagcttcgcggtggatgccccag

SEQ ID NO: 127 - DNA, 30 bp, Artificial Sequence
  Internal control tag sequence derivated from Pseudomonas aeruginosa
gcataggacggagcttcgcggtggatgccc

SEQ ID NO: 128 - DNA, 20 bp, Artificial Sequence
  Internal control tag sequence derivated from Pseudomonas aeruginosa
ggacggagcttcgcggtgga

SEQ ID NO: 129 - DNA, 35 bp, Artificial Sequence
  Internal control tag sequence derivated from Pseudomonas aeruginosa
gcgccgccgaacaggcctaccttgccgcccttggc

SEQ ID NO: 130 - DNA, 20 bp, Artificial Sequence
  Internal control tag sequence derivated from Pseudomonas aeruginosa
atgatccggcccagggtcgc

SEQ ID NO: 131 - DNA, 20 bp, Propionibacterium acnes
catgccgcgaacgacatcct

SEQ ID NO: 132 - DNA, 20 bp, Propionibacterium acnes
ggctgtagtgggagaagaac

SEQ ID NO: 133 - DNA, 20 bp, Proteus mirabilis
acctacgttctcacctgcac

SEQ ID NO: 134 - DNA, 20 bp, Proteus mirabilis
ttcacgttttgtaccacgca

SEQ ID NO: 135 - DNA, 18 bp, Proteus mirabilis
cacgttttgtaccacgca

SEQ ID NO: 136 - DNA, 20 bp, Proteus mirabilis
cagtacttgtccacgttcga

SEQ ID NO: 137 - DNA, 19 bp, Proteus mirabilis
caaatttgttgtgtgggtt

SEQ ID NO: 138 - DNA, 17 bp, Proteus mirabilis
caaatttgttgtgtggg

SEQ ID NO: 139 - DNA, 18 bp, Proteus mirabilis
agcctttgaagaatggag

SEQ ID NO: 140 - DNA, 18 bp, Proteus mirabillis
ctacgttctcacctgcac

SEQ ID NO: 141 - DNA, 17 bp, Proteus mirabillis
ctacgttctcacctgca

SEQ ID NO: 142 - DNA, 20 bp, Providencia rettgeri
acctggttttgccagtactt

SEQ ID NO: 143 - DNA, 18 bp, Providencia rettgeri
acctggttttgccagtac

SEQ ID NO: 144 - DNA, 17 bp, Providencia rettgeri
acctggttttgccagta

SEQ ID NO: 145 - DNA, 20 bp, Pseudomonas aeruginosa
gcagcaggataccaacgttc

SEQ ID NO: 146 - DNA, 17 bp, Pseudomonas aeruginosa
cagcaggataccaacgt

SEQ ID NO: 147 - DNA, 16 bp, Pseudomonas aeruginosa
agcaggataccaacgt

SEQ ID NO: 148 - DNA, 20 bp, Pseudomonas aeruginosa
gccacgctctacgtcttcac

SEQ ID NO: 149 - DNA, 18 bp, Pseudomonas aeruginosa
gccacgctctacgtcttc

SEQ ID NO: 150 - DNA, 17 bp, Pseudomonas aeruginosa
gccacgctctacgtctt

SEQ ID NO: 151 - DNA, 16 bp, Pseudomonas aeruginosa
gccacgctctacgtct

SEQ ID NO: 152 - DNA, 18 bp, Salmonella choleraesuis
ggcttgatggtgcccggc

SEQ ID NO: 153 - DNA, 17 bp, Salmonella choleraesuis
ggcttgatggtgcccgg

SEQ ID NO: 154 - DNA, 16 bp, Salmonella choleraesuis
ggcttgatggtgcccg

SEQ ID NO: 155 - DNA, 19 bp, Artificial Sequence
  Serratia sp.
ctttgctcaggatgtacac

SEQ ID NO: 156 - DNA, 18 bp, Artificial Sequence
  Serratia sp.
ctttgctcaggatgtaca

SEQ ID NO: 157 - DNA, 19 bp, Serratia liquefaciens
cgatgtcttcacgcttgat

SEQ ID NO: 158 - DNA, 18 bp, Serratia liquefaciens
cgatgtcttcacgcttga

SEQ ID NO: 159 - DNA, 20 bp, Serratia liquefaciens
cacttctgagtcgaacttgg

SEQ ID NO: 160 - DNA, 18 bp, Serratia liquefaciens
cacttctgagtcgaactt

SEQ ID NO: 161 - DNA, 19 bp, Serratia marcescens
cagattcgaactgggtgtg

SEQ ID NO: 162 - DNA, 18 bp, Serratia marcescens
agattcgaactgggtgtg

SEQ ID NO: 163 - DNA, 18 bp, Serratia marcescens
catctttgctcaggatgt

SEQ ID NO: 164 - DNA, 17 bp, Serratia marcescens
atctttgctcaggatgt

SEQ ID NO: 165 - DNA, 20 bp, Serratia marcescens
ttcatctttgctcaggatgt

SEQ ID NO: 166 - DNA, 16 bp, Serratia marcescens
atctttgctcaggatg

SEQ ID NO: 167 - DNA, 19 bp, Serratia marcescens
tgtgacgaccaccttcatc

SEQ ID NO: 168 - DNA, 18 bp, Serratia marcescens
tgtgacgaccaccttcat

SEQ ID NO: 169 - DNA, 20 bp, Streptococcus agalactiae
aacgttgtcccctgcaagac

SEQ ID NO: 170 - DNA, 18 bp, Streptococcus agalactiae
aacgttgtcccctgcaag

SEQ ID NO: 171 - DNA, 17 bp, Streptococcus agalactiae
aacgttgtcccctgcaa

SEQ ID NO: 172 - DNA, 16 bp, Streptococcus agalactiae
aacgttgtcccctgca

SEQ ID NO: 173 - DNA, 20 bp, Streptococcus agalactiae
aacaccacgaagaagaacac

SEQ ID NO: 174 - DNA, 20 bp, Streptococcus agalactiae
tggtttagcaagaacttgac

SEQ ID NO: 175 - DNA, 17 bp, Streptococcus agalactiae
gtttagcaagaacttga

SEQ ID NO: 176 - DNA, 20 bp, Streptococcus agalactiae
taaacttcacctttaaattt

SEQ ID NO: 177 - DNA, 20 bp, Artificial Sequence
  Streptococcus anginosus / Streptococcus constellatus
gaagaagaacccctacgtta

SEQ ID NO: 178 - DNA, 20 bp, Artificial Sequence
  Streptococcus anginosus / Streptococcus constellatus
caagaacttgtccacgttcg

SEQ ID NO: 179 - DNA, 18 bp, Artificial Sequence
  Streptococcus anginosus / Streptococcus constellatus
caagaacttgtccacgtt

SEQ ID NO: 180 - DNA, 20 bp, Streptococcus bovis
aagaacaccaacgttatccc

SEQ ID NO: 181 - DNA, 18 bp, Streptococcus bovis
tcacgttggataccacga

SEQ ID NO: 182 - DNA, 20 bp, Streptococcus mutans
tccaccttcctctttagtaa

SEQ ID NO: 183 - DNA, 17 bp, Streptococcus mutans
accttcctctttagtaa

SEQ ID NO: 184 - DNA, 20 bp, Streptococcus salivarius
ctccggcaataccttcgtca

SEQ ID NO: 185 - DNA, 17 bp, Streptococcus salivarius
ctccggcaataccttcg

SEQ ID NO: 186 - DNA, 20 bp, Streptococcus salivarius
aagaacaccgacgttatctc

SEQ ID NO: 187 - DNA, 20 bp, Streptococcus salivarius
gaaccaggtgcagccaatac

SEQ ID NO: 188 - DNA, 18 bp, Streptococcus salivarius
aaccaggtgcagccaata

SEQ ID NO: 189 - DNA, 20 bp, Streptococcus sanguinis
tacgttgtcccctgcaagac

SEQ ID NO: 190 - DNA, 18 bp, Streptococcus sanguinis
tacgttgtcccctgcaag

SEQ ID NO: 191 - DNA, 17 bp, Streptococcus sanguinis
tacgttgtcccctgcaa

SEQ ID NO: 192 - DNA, 20 bp, Streptococcus suis
ctggtttagagataacttga

SEQ ID NO: 193 - DNA, 18 bp, Streptococcus suis
ggtttagagataacttga

SEQ ID NO: 194 - DNA, 20 bp, Vibrio vulnificus
acgtagtagggcaccaacgt

SEQ ID NO: 195 - DNA, 18 bp, Vibrio vulnificus
acgtagtagggcaccaac

SEQ ID NO: 196 - DNA, 18 bp, Vibrio vulnificus
acgtagtagcgcaccaac

SEQ ID NO: 197 - DNA, 20 bp, Yersinia enterocolitica
tmgaacctggtttagcaaga

SEQ ID NO: 198 - DNA, 18 bp, Yersinia enterocolitica
tagaacctggtttagcaa

SEQ ID NO: 199 - DNA, 18 bp, Yersinia enterocolitica
tcgaacctggtttagcaa

SEQ ID NO: 200 - DNA, 20 bp, Artificial Sequence
  Yersinia pestis / Yersinia pseudotuberculosis
ggtttgatagaacctggttt

SEQ ID NO: 201 - DNA, 18 bp, Artificial Sequence
  Yersinia pestis / Yersinia pseudotuberculosis
ggtttgatagaacctggt

SEQ ID NO: 202 - DNA, 20 bp, Artificial Sequence
  Yersinia pestis / Yersinia pseudotuberculosis
cacgctgaacatcgtcacgc

SEQ ID NO: 203 - DNA, 17 bp, Artificial Sequence
  Yersinia pestis / Yersinia pseudotuberculosis
cgctgaacatcgtcacg

SEQ ID NO: 204 - DNA, 19 bp, Citrobacter freundii complexe
gacagaagttcacgaactt

SEQ ID NO: 205 - DNA, 18 bp, Citrobacter freundii complexe
acagaagttcacgaactt

SEQ ID NO: 206 - DNA, 20 bp, Citrobacter freundii
ttccatttctaccagttcca

SEQ ID NO: 207 - DNA, 18 bp, Citrobacter freundii
tccatttctaccagttcc

SEQ ID NO: 208 - DNA, 17 bp, Citrobacter freundii
ccatttctaccagttcc

SEQ ID NO: 209 - DNA, 20 bp, Citrobacter freundii
agtgtcgtcgcccgggaaat

SEQ ID NO: 210 - DNA, 18 bp, Citrobacter freundii
tgtcgtcgcccgggaaat

SEQ ID NO: 211 - DNA, 17 bp, Citrobacter freundii
gtcgtcgcccgggaaat

SEQ ID NO: 212 - DNA, 20 bp, Citrobacter freundii
cacgaacgatcggagtgtcg

SEQ ID NO: 213 - DNA, 20 bp, Citrobacter koseri
gcagttcacgcacttccatc

SEQ ID NO: 214 - DNA, 18 bp, Citrobacter koseri
gcagttcacgcacttcca

SEQ ID NO: 215 - DNA, 19 bp, Artificial Sequence
  Citrobacter koserii / Enterobacter sakazakii
cgcacttccatctcaacca

SEQ ID NO: 216 - DNA, 18 bp, Enterobacter aerogenes
cgaacttccatctcaacc

SEQ ID NO: 217 - DNA, 20 bp, Enterobacter cloacae
tgtgctcacgagtctgaggc

SEQ ID NO: 218 - DNA, 18 bp, Enterobacter cloacae
tgctcacgagtctgaggc

SEQ ID NO: 219 - DNA, 17 bp, Enterobacter cloacae
tgctcacgagtctgagg

SEQ ID NO: 220 - DNA, 20 bp, Enterobacter cloacae
tctctaccagttccagcagc

SEQ ID NO: 221 - DNA, 18 bp, Enterobacter cloacae
tctctaccagttccagca

SEQ ID NO: 222 - DNA, 20 bp, Enterobacter cloacae
cgtcgcctgggaaatcgtac

SEQ ID NO: 223 - DNA, 17 bp, Enterobacter cloacae complexe
gaaccacgaacgattgg

SEQ ID NO: 224 - DNA, 20 bp, Enterobacter sakazakii
gtcgtactgagacagcagct

SEQ ID NO: 225 - DNA, 18 bp, Klebsiella oxytoca
aagaatccaggaagccag

SEQ ID NO: 226 - DNA, 18 bp, Klebsiella pneumoniae
aggtatccaggtggccag

SEQ ID NO: 227 - DNA, 20 bp, Listeria monocytogenes
gtggagtaatcgaacctggt

SEQ ID NO: 228 - DNA, 18 bp, Listeria monocytogenes
tggagtaatcgaacctgg

SEQ ID NO: 229 - DNA, 17 bp, Listeria monocytogenes
ggagtaatcgaacctgg

SEQ ID NO: 230 - DNA, 20 bp, Listeria monocytogenes
aaaacataagtttcagcttt

SEQ ID NO: 231 - DNA, 20 bp, Pasteurella pneumotropica
attcgaagtcagtgtgtggc

SEQ ID NO: 232 - DNA, 20 bp, Pasteurella pneumotropica
gccacacactgacttcgaat

SEQ ID NO: 233 - DNA, 20 bp, Pasteurella pneumotropica
ttcatcttttgataatacgt

SEQ ID NO: 234 - DNA, 20 bp, Pasteurella pneumotropica
acgtattatcaaaagatgaa

SEQ ID NO: 235 - DNA, 20 bp, Pasteurella pneumotropica
tgaagaatggcgtatgacga

SEQ ID NO: 236 - DNA, 18 bp, Pasteurella pneumotropica
aagaatggcgtatgacga

SEQ ID NO: 237 - DNA, 17 bp, Pasteurella pneumotropica
agaatggcgtatgacga

SEQ ID NO: 238 - DNA, 40 bp, Artificial Sequence
  Internal control tag sequence derivated from Pseudomonas aeruginosa
gtgcgcaccttccaagacctgattctcgccctgcagaact

SEQ ID NO: 239 - DNA, 30 bp, Artificial Sequence
  Internal control tag sequence derivated from Pseudomonas aeruginosa
accttccaagacctgattctcgccctgcag

SEQ ID NO: 240 - DNA, 35 bp, Artificial Sequence
  Internal control tag sequence derivated from Pseudomonas aeruginosa
ccccaaccgcctgcagcactactaccagtttcagg

SEQ ID NO: 241 - DNA, 20 bp, Salmonella choleraesuis
tgtgctcacgggtctgcggc

SEQ ID NO: 242 - DNA, 19 bp, Salmonella choleraesuis
taagaatccaggaagccag

SEQ ID NO: 243 - DNA, 18 bp, Salmonella choleraesuis
taagaatccaggaagcca

SEQ ID NO: 244 - DNA, 20 bp, Staphylococcus aureus
cagtatgtggtgtaattgaa

SEQ ID NO: 245 - DNA, 17 bp, Staphylococcus aureus
cagtatgtggtgtaatt

SEQ ID NO: 246 - DNA, 18 bp, Staphylococcus aureus
tcgtcttttgataatacg

SEQ ID NO: 247 - DNA, 17 bp, Staphylococcus aureus
cgtcttttgataatacg

SEQ ID NO: 248 - DNA, 20 bp, Staphylococcus epidermidis
tggtgtaatagaaccaggag

SEQ ID NO: 249 - DNA, 17 bp, Staphylococcus epidermidis
tgtaatagaaccaggag

SEQ ID NO: 250 - DNA, 18 bp, Staphylococcus epidermidis
ggtgtaatagaaccagga

SEQ ID NO: 251 - DNA, 20 bp, Staphylococcus epidermidis
gcgatagttagtgaagaatg

SEQ ID NO: 252 - DNA, 18 bp, Staphylococcus epidermidis
gcgatagttagtgaagaa

SEQ ID NO: 253 - DNA, 20 bp, Staphylococcus haemolyticus
ttgtgtgaggtgtgattgaa

SEQ ID NO: 254 - DNA, 21 bp, Staphylococcus haemolyticus
tatacgtctgctttaaatttt

SEQ ID NO: 255 - DNA, 20 bp, Staphylococcus haemolyticus
cgtctttagataaaacgtat

SEQ ID NO: 256 - DNA, 18 bp, Staphylococcus hominis
tacgtctgctttgaattt

SEQ ID NO: 257 - DNA, 20 bp, Staphylococcus hominis
aaacatatacgtctgctttg

SEQ ID NO: 258 - DNA, 20 bp, Staphylococcus hominis
aaacgtatacgtctgctttg

SEQ ID NO: 259 - DNA, 20 bp, Staphylococcus hominis
catcttttgataaaacgtat

SEQ ID NO: 260 - DNA, 20 bp, Staphylococcus hominis
catcttttgataaaacatat

SEQ ID NO: 261 - DNA, 20 bp, Staphylococcus hominis
cttcatcttttgataaaacg

SEQ ID NO: 262 - DNA, 20 bp, Staphylococcus saccharolyticus
ttagtgtgtggtgtgattga

SEQ ID NO: 263 - DNA, 18 bp, Staphylococcus saccharolyticus
tagtgtgtggtgtgattg

SEQ ID NO: 264 - DNA, 20 bp, Staphylococcus saccharolyticus
aaaacgtaaacttcagcttt

SEQ ID NO: 265 - DNA, 20 bp, Staphylococcus saprophyticus
cgtaaacatccgctttgaat

SEQ ID NO: 266 - DNA, 18 bp, Staphylococcus saprophyticus
cgtaaacatccgctttga

SEQ ID NO: 267 - DNA, 18 bp, Staphylococcus warneri
gtgtaattgaaccaggag

SEQ ID NO: 268 - DNA, 17 bp, Staphylococcus warneri
gtgtaattgaaccagga

SEQ ID NO: 269 - DNA, 20 bp, Staphylococcus warneri
attttgtatgtggtgtaatt

SEQ ID NO: 270 - DNA, 20 bp, Staphylococcus warneri
cgtaaacttccgctttgaat

SEQ ID NO: 271 - DNA, 17 bp, Staphylococcus warneri
gtaaacttccgctttga

SEQ ID NO: 272 - DNA, 18 bp, Staphylococcus warneri
gtgacgtccaccttcgtc

SEQ ID NO: 273 - DNA, 16 bp, Staphylococcus warneri
gtgacgtccaccttcg

SEQ ID NO: 274 - DNA, 20 bp, Streptococcus agalactiae
gcgcctgaatcaatcaattt

SEQ ID NO: 275 - DNA, 20 bp, Streptococcus bovis
tgcaatttcaagaccttgtt

SEQ ID NO: 276 - DNA, 20 bp, Streptococcus canis
gcaccagaatcaattaattt

SEQ ID NO: 277 - DNA, 20 bp, Streptococcus dysgalactiae
ccccaagcgcagcagcgtaa

SEQ ID NO: 278 - DNA, 18 bp, Streptococcus dysgalactiae
ccaagcgcagcagcgtaa

SEQ ID NO: 279 - DNA, 17 bp, Streptococcus dysgalactiae
caagcgcagcagcgtaa

SEQ ID NO: 280 - DNA, 16 bp, Streptococcus dysgalactiae
aagcgcagcagcgtaa

SEQ ID NO: 281 - DNA, 20 bp, Streptococcus dysgalactiae
aatttcaagtccttgttctc

SEQ ID NO: 282 - DNA, 17 bp, Streptococcus dysgalactiae
ttcaagtccttgttctc

SEQ ID NO: 283 - DNA, 20 bp, Streptococcus gordonii
aatcaatttcccagcaattt

SEQ ID NO: 284 - DNA, 20 bp, Streptococcus mitis
aatcaattttcctgcaatct

SEQ ID NO: 285 - DNA, 20 bp, Streptococcus oralis
aatcaattttccagcaattt

SEQ ID NO: 286 - DNA, 20 bp, Streptococcus pneumoniae
gcagcataagctggatcaag

SEQ ID NO: 287 - DNA, 20 bp, Streptococcus pneumoniae
aatcaattttcccgcaatct

SEQ ID NO: 288 - DNA, 20 bp, Streptococcus pneumoniae
aaccaacatggctatctccg

SEQ ID NO: 289 - DNA, 20 bp, Streptococcus pyogenes
ccccaagcgcagcagcataa

SEQ ID NO: 290 - DNA, 17 bp, Streptococcus pyogenes
ccccaagcgcagcagca

SEQ ID NO: 291 - DNA, 17 bp, Streptococcus pyogenes
acaaccagatcaaccgc

SEQ ID NO: 292 - DNA, 18 bp, Streptococcus pyogenes
caacaaccagatcaaccg

SEQ ID NO: 293 - DNA, 20 bp, Streptococcus sanguinis
gcacctgagtcaatcagctt

SEQ ID NO: 294 - DNA, 20 bp, Artificial Sequence
  Aspergillus sp.
aagtcacggtgaccgggggc

SEQ ID NO: 295 - DNA, 17 bp, Artificial Sequence
  Aspergillus sp.
tcacggtgaccgggggc

SEQ ID NO: 296 - DNA, 19 bp, Aspergillus flavus
gctcacgggtctgaccatc

SEQ ID NO: 297 - DNA, 20 bp, Aspergillus fumigatus
atcgtgttagctacagcacc

SEQ ID NO: 298 - DNA, 20 bp, Aspergillus fumigatus
gatgagctgcttgacaccga

SEQ ID NO: 299 - DNA, 18 bp, Aspergillus fumigatus
atgagctgcttgacaccg

SEQ ID NO: 300 - DNA, 20 bp, Aspergillus nidulans
gcaacaatgagctgacggac

SEQ ID NO: 301 - DNA, 18 bp, Aspergillus nidulans
caacaatgagctgacgga

SEQ ID NO: 302 - DNA, 18 bp, Aspergillus niger
atgagctggcggacaccg

SEQ ID NO: 303 - DNA, 18 bp, Aspergillus niger
caacgatgagctggcgga

SEQ ID NO: 304 - DNA, 20 bp, Aspergillus terreus
gagggtgaaggcaagcagag

SEQ ID NO: 305 - DNA, 18 bp, Aspergillus terreus
agggtgaaggcaagcaga

SEQ ID NO: 306 - DNA, 20 bp, Candida albicans
  misc_feature (8)..(8): n is inosine
gttggtgnatggttcaatca

SEQ ID NO: 307 - DNA, 18 bp, Candida albicans
ttggtggatggttcaatc

SEQ ID NO: 308 - DNA, 17 bp, Candida albicans
tggtggatggttcaatc

SEQ ID NO: 309 - DNA, 20 bp, Candida albicans
  misc_feature (14)..(14): n is inosine
accagtaactttancggatt

SEQ ID NO: 310 - DNA, 20 bp, Artificial Sequence
  Candida albicans / Candida dublininensis
ctttaccggatttggtttcc

SEQ ID NO: 311 - DNA, 20 bp, Artificial Sequence
  Candida albicans / Candida dublininensis
ccttaccggatttggtttcc

SEQ ID NO: 312 - DNA, 18 bp, Artificial Sequence
  Candida albicans / Candida dublininensis
ttaccggatttggtttcc

SEQ ID NO: 313 - DNA, 20 bp, Artificial Sequence
  Candida albicans / Candida dublininensis
ggtcttaccagtaactttac

SEQ ID NO: 314 - DNA, 19 bp, Artificial Sequence
  Candida albicans / Candida dublininensis
gtcttaccagtaactttac

SEQ ID NO: 315 - DNA, 18 bp, Artificial Sequence
  Candida albicans / Candida dublininensis
tggtctggttggtggttc

SEQ ID NO: 316 - DNA, 20 bp, Candida dubliniensis
  misc_feature (14)..(14): n is inosine
gttggtggaagctncaatca

SEQ ID NO: 317 - DNA, 18 bp, Candida dubliniensis
ttggtggaagcttcaatc

SEQ ID NO: 318 - DNA, 18 bp, Candida glabrata
cgatttcagcgaatctgg

SEQ ID NO: 319 - DNA, 20 bp, Candida glabrata
tgtaccaggaagcgttggtg

SEQ ID NO: 320 - DNA, 18 bp, Candida glabrata
taccaggaagcgttggtg

SEQ ID NO: 321 - DNA, 18 bp, Candida krusei
ggttggtctgacaggtgg

SEQ ID NO: 322 - DNA, 18 bp, Candida krusei
taatggcttttcggttgg

SEQ ID NO: 323 - DNA, 17 bp, Candida krusei
taatggcttttcggttg

SEQ ID NO: 324 - DNA, 20 bp, Candida parapsilosis
atgggacagctttagggttg

SEQ ID NO: 325 - DNA, 22 bp, Candida parapsilosis
accagctttagtttccttttcc

SEQ ID NO: 326 - DNA, 20 bp, Candida parapsilosis
ccttaccagctttagtttcc

SEQ ID NO: 327 - DNA, 18 bp, Candida parapsilosis
ccttaccagctttagttt

SEQ ID NO: 328 - DNA, 20 bp, Candida tropicalis
cttggtttctttttcccaac

SEQ ID NO: 329 - DNA, 18 bp, Candida tropicalis
cttggtttctttttccca

SEQ ID NO: 330 - DNA, 17 bp, Candida tropicalis
cttggtttctttttccc

SEQ ID NO: 331 - DNA, 20 bp, Candida tropicalis
ttggtcttgaaggtggttca

SEQ ID NO: 332 - DNA, 18 bp, Candida tropicalis
ggtcttgaaggtggttca

SEQ ID NO: 333 - DNA, 17 bp, Candida tropicalis
gtcttgaaggtggttca

SEQ ID NO: 334 - DNA, 40 bp, Artificial Sequence
  Internal control tag sequence derivated from pACYC184
ttgggcgctgccggcacctgtcctacgagttgcatgataa

SEQ ID NO: 335 - DNA, 30 bp, Artificial Sequence
  Internal control tag sequence derivated from pACYC184
ctgccggcacctgtcctacgagttgcatga

SEQ ID NO: 336 - DNA, 20 bp, Artificial Sequence
  Internal control tag sequence derivated from pACYC184
ccggcacctgtcctacgagt

SEQ ID NO: 337 - DNA, 20 bp, Artificial Sequence
  Internal control tag sequence derivated from pACYC184
gcgtgggtatggtggcaggc

SEQ ID NO: 338 - DNA, 35 bp, Artificial Sequence
  Internal control tag sequence derivated from pACYC184
cggcagcggtgcggactgttgtaactcagaataag

SEQ ID NO: 339 - DNA, 17 bp, Bacteroides fragilis
atcgaaactggtgttat

SEQ ID NO: 340 - DNA, 17 bp, Bacteroides fragilis
cctcggtttgggtgaag

SEQ ID NO: 341 - DNA, 17 bp, Bacteroides fragilis
aatcagttgtaacaggt

SEQ ID NO: 342 - DNA, 20 bp, Brucella melitensis
cgtcggcatcaaggcgacga

SEQ ID NO: 343 - DNA, 18 bp, Brucella melitensis
tcggcatcaaggcgacga

SEQ ID NO: 344 - DNA, 17 bp, Brucella melitensis
cggcatcaaggcgacga

SEQ ID NO: 345 - DNA, 20 bp, Brucella melitensis
cgaagaccacggttaccggc

SEQ ID NO: 346 - DNA, 17 bp, Brucella melitensis
aagaccacggttaccgg

SEQ ID NO: 347 - DNA, 20 bp, Burkholderia cepacia
cggcatcgtgaaggtcggcg

SEQ ID NO: 348 - DNA, 17 bp, Burkholderia cepacia
ggcatcgtgaaggtcgg

SEQ ID NO: 349 - DNA, 18 bp, Artificial Sequence
  Escherichia coli / Shigella sp.
agcaggaacggcttgtca

SEQ ID NO: 350 - DNA, 18 bp, Artificial Sequence
  Escherichia coli / Shigella sp.
gagaatacgtcttcgatc

SEQ ID NO: 351 - DNA, 20 bp, Artificial Sequence
  Escherichia coli / Shigella sp.
acttcttcaccaactttgat

SEQ ID NO: 352 - DNA, 18 bp, Artificial Sequence
  Escherichia coli / Shigella sp.
cttcttcaccaactttga

SEQ ID NO: 353 - DNA, 35 bp, Artificial Sequence
  Internal control tag sequence derivated from pACYC184
gcgccgccctataccttgtctgcctccccgcgttg

SEQ ID NO: 354 - DNA, 40 bp, Artificial Sequence
  Internal control tag sequence derivated from pACYC184
gacgaccatcagggacagcttcaaggatcgctcgcggctc

SEQ ID NO: 355 - DNA, 30 bp, Artificial Sequence
  Internal control tag sequence derivated from pACYC184
accatcagggacagcttcaaggatcgctcg

SEQ ID NO: 356 - DNA, 18 bp, Stenotrophomonas maltophilia
ccgtccggtgcagaagac

SEQ ID NO: 357 - DNA, 16 bp, Stenotrophomonas maltophilia
ccgtccggtgcagaag

SEQ ID NO: 358 - DNA, 17 bp, Streptomyces avermitilis
tcgtggcacggtcgtca

SEQ ID NO: 359 - DNA, 25 bp, Streptomyces avermitilis
tcgtggcacggtcgtcaccggtcgt

SEQ ID NO: 360 - DNA, 30 bp, Streptomyces avermitilis
tcgtggcacggtcgtcaccggtcgtatcga

SEQ ID NO: 361 - DNA, 19 bp, Streptomyces avermitilis
tggcacggtcgtcaccggt

SEQ ID NO: 362 - DNA, 20 bp, Streptomyces avermitilis
cgtcgacatcgtcggtatca

SEQ ID NO: 363 - DNA, 30 bp, Streptomyces avermitilis
cgtcgacatcgtcggtatcaagaccgagaa

SEQ ID NO: 364 - DNA, 21 bp, Klebsiella pneumoniae
tataggtatccaggtggccag

SEQ ID NO: 365 - DNA, 35 bp, Artificial Sequence
  Internal control tag sequence derivated from Pseudomonas aeruginosa
ggccgaggttgatgcgattgaccacggtgcccttg

SEQ ID NO: 366 - DNA, 16 bp, Burkholderia cepacia
ggcatcgtgaaggtcg

SEQ ID NO: 367 - DNA, 19 bp, Burkholderia cepacia
tcaagccgacggtgaagac

SEQ ID NO: 368 - DNA, 25 bp, Artificial Sequence
  Escherichia coli / Shigella sp.
gagcgtgcgattgacaagccgttcc

SEQ ID NO: 369 - DNA, 24 bp, Artificial Sequence
  Escherichia coli / Shigella sp.
ttctccatctccggtcgtggtacc

SEQ ID NO: 370 - DNA, 23 bp, Artificial Sequence
  Escherichia coli / Shigella sp.
catcaaagttggtgaagaagttg

SEQ ID NO: 371 - DNA, 18 bp, Artificial Sequence
  Escherichia coli / Shigella sp.
tcaaagttggtgaagaag

SEQ ID NO: 372 - DNA, 18 bp, Stenotrophomonas maltophilia
gagcgcggcgtgatcaag

SEQ ID NO: 373 - DNA, 19 bp, Stenotrophomonas maltophilia
ggcgacgaaatcgaaatcg

SEQ ID NO: 374 - DNA, 18 bp, Stenotrophomonas maltophilia
gaagaccaccgtgaccgg

SEQ ID NO: 375 - DNA, 23 bp, Bacteria
  misc_feature (12)..(12): n is inosine
actggygttganatgttccgyaa

SEQ ID NO: 376 - DNA, 23 bp, Bacteria
  misc_feature (9)..(9): n is inosine
acgtcagtngtacggaartagaa

SEQ ID NO: 377 - DNA, 18 bp, Artificial Sequence
  Escherichia coli / Shigella sp.
tgggaagcgaaaatcctg

SEQ ID NO: 378 - DNA, 19 bp, Artificial Sequence
  Escherichia coli / Shigella sp.
cagtacaggtagacttctg

(19) United States
(12) Patent Application Publication
(10) Pub. No.: US 2011/0151453 A1
(43) Pub. Date: Jun. 23, 2011

(54) NUCLEIC ACID SEQUENCES AND COMBINATION THEREOF FOR SENSITIVE AMPLIFICATION AND DETECTION OF BACTERIAL AND FUNGAL SEPSIS PATHOGENS

(76) Inventors: Michel G. Bergeron, Quebec (CA); Maurice Boissinot, St-Augustin-de-Desmaures (CA); Dominique Boudreau, Quebec (CA); Richard Giroux, Quebec (CA); Ann Hulet-Sky, Quebec (CA); Isabelle Martineau, Quebec (CA); Catherine Ouellet, Levis (CA)

(21) Appl. No.: 12/668,622

(22) PCT Filed: Jul. 11, 2008
(86) PCT No.: PCT/CA2008/001298

§ 371 Date: Jun. 18, 2010

Related U.S. Application Data
(60) Provisional application No. 60/929,749, filed on Jul. 11, 2007.

(51) Int. Cl. C12Q 1/68; C07H 21/00; C40B 40/06

(52) U.S. Cl. 435/6.11; 435/6.15; 536/23.1; 506/16

(57) ABSTRACT

The present invention relates to methods of detection, as well as assays, reagents and kits for the specific detection of clinically important bacterial and fungal species. The present invention allows for the specific detection of nucleic acids of each of these pathogens in a single assay.

### FIELD OF THE INVENTION

The present invention provides nucleic acid sequences and combinations for sensitive amplification and detection of bacterial and fungal pathogens. More particularly, the present invention relates to methods of detection of bacterial and fungal pathogens associated with bloodstream infection as well as assays, reagents and kits for their specific detection.

### BACKGROUND OF THE INVENTION

Infectious diseases are still a major cause of death worldwide. However, of the millions of microbial species inhabiting our planet, only few hundreds species are recognized as human pathogens, among which over 500 bacteria and around 300 fungi (Taylor, L. H. et al., 2001, Philos. Trans. R. Soc. Lond., B, Biol. Sci. 356:983-989). Since proper therapeutic intervention differs depending upon the species responsible for the disease, detection and identification of these microbes are key factors for controlling infections. Molecular methods relying on the detection of microbial nucleic acids offer a rapid alternative to the slower traditional culture-based techniques for the diagnosis of infectious diseases. However, using single specific molecular assays for each bacterial species is cumbersome and could exhaust precious clinical samples. One solution is to perform simultaneous tests on a single sample by combining many primers to amplify target nucleic acids in a multiplex fashion such as in the multiplex polymerase chain reaction (multiplex PCR) (Chamberlain, J. S. et al., 1988, Nucleic Acids Res. 16:11141-11156). The drawback is that such complexification of the target amplification reaction creates more opportunities to form incorrect amplicons hence reducing the yield and specificity of the amplification process. Even with careful primer design, it is difficult to overcome these limitations. The problem is even harder when very low levels of target template nucleic acids are present in the sample.

Bloodstream infections represent one of the most challenging situation since often, very few micro-organisms are present per milliliter of blood (Peters, R. P. et al., 2004, Lancet Infect Dis. 4:751-760) and these blood infections can be caused by hundreds of genetically different bacterial and fungal species.

A further limitation of widespread nucleic acid diagnostic methods is the detection technique required to detect and identify the amplification product. Detection technologies exist for real-time monitoring of the nucleic acid amplification reaction (Wittwer, C. T. et al. 1997, BioTechniques 22:130-139). However, these homogeneous methods have limited multiplexing capabilities due to the overlap between the emission spectra of the fluorescent molecules available for labelling nucleic acids. A combination of real-time fluorescence detection and post-amplification melting curve analysis detection techniques can increase the multiplexing power but so far, practical applications have been restricted to distinguishing only around 20 different targets (LightCycler® SeptiFast Test, Roche). Separation of nucleic acid amplification products by agarose gel electrophoresis followed by staining with a fluorescent intercalator dye is limited to distinguishing amplicons of different length and prone to carryover contaminations. Sequencing methods are currently too slow or too costly for clinical diagnostics. Post-amplification hybridization to different probes physically addressed onto solid (or semi-solid gels) surfaces offer very high multiplexing capability (Bodrossy, L. and Sessitsch, A., 2004, Curr. Opin. Microbiol. 7:245-254; Loy, A. and Bodrossy, L., 2006, Clin. Chim. Acta 363:106-119). However, obtaining specific and sensitive probe sequences represent a challenge due to the lack of understanding of hybridization behaviour of oligonucleotide probes which are affected by immobilization to solid support, steric hindrance, dissociation of mixed targets, etc. Nonequilibrium thermal dissociation models cannot efficiently predict which probe sequence will interact efficiently and specifically with its matched complementary sequence and under which stringency conditions (Pozhitkov, A. E. et al., 2007, Nucleic Acids Res. 35:e70).

There is thus a need for improved reagents and assays allowing the specific and sensitive detection of sepsis-associated bacterial and fungal pathogens.

The present invention seeks to meet these and other needs.

### SUMMARY OF THE INVENTION

The present invention provides nucleic acid sequences and combinations for sensitive amplification and detection of bacterial and fungal pathogens. More particularly, the present invention relates to methods of detection of bacterial and fungal pathogens associated with bloodstream infection as well as assays, reagents and kits for their specific detection.

Aspects of the invention therefore relate to primers, probes, combinations of primers or probes or combination of primers and probes allowing the specific detection of bacterial and fungal pathogens.

The primers and probes of the present invention have especially been chosen to target the most important human pathogens associated with bloodstream infection included in but not limited to the list of Table 4. The present invention thus provides oligonucleotides of from 10 to 50 nucleotides long which may be capable of specific binding to a pathogen selected from the group consisting of those listed in Table 4. These oligonucleotides may be used individually, or collectively (in groups or subgroups) in the methods and kits of the present invention.

In accordance with the present invention, some of the oligonucleotides of the present invention may be capable of binding (or preferably binds) to a genetic material of one pathogen species.

To the best of the Applicant's knowledge, the combinations of primers and/or probes presented herein have not been previously described. In accordance with an embodiment of the invention, detection of the above mentioned bacterial and fungal pathogens may be performed simultaneously. In accordance with a further embodiment of the invention, detection of the above mentioned bacterial and fungal pathogens may be performed in parallel. Of course, if desired, the detection of the above mentioned bacterial and fungal pathogens may be performed separately (i.e., in separate test tubes and/or in separate experiments).

Primers and probes sequences which are the object of this invention are derived from evolutionary conserved protein-coding genes sequence database generated as described in international patent application NO. PCT/CA00/01150 filed on Sep. 28, 2000 and published on Apr. 5, 2001 under no. WO 2001/023604A2. The present invention, discloses oligonucleotide combinations optimized to be used under uniform conditions of temperature and reagents/buffer solutions.

Some aspects of the invention also relate to methods of detection. The methods of detection may be carried out by amplification of the genetic material, by hybridization of the genetic material with oligonucleotides or by a combination of amplification and hybridization.

A significant advantage of the present invention is that the amplification step may be performed under similar or uniform amplification conditions for each pathogen species. As such, amplification of each pathogen species may be performed simultaneously.

Another significant advantage of the invention is that hybridization may also be performed under similar or uniform hybridization conditions.

Detection of the genetic material may also advantageously be performed under uniform conditions.

Thus, aspects of the invention relates to methods for detecting and/or identifying a pathogen which may include the steps of contacting a sample comprising or suspected of comprising a genetic material originating from the pathogen and; —the oligonucleotide or combination of oligonucleotides under suitable conditions of hybridization, amplification and/or detection.

More specifically, the present invention relates to optimal combinations of amplification primer sequences for efficient multiplex broad-spectrum nucleic acid amplification reaction under uniform conditions of temperature and reagents/buffer solutions for all primer combinations. These combinations may be particularly useful for diagnostic, identification and detection purposes.

Further aspects of the invention relates to combinations of the nucleic acid sequences described herein as well as kits, arrays and methods of detection.

The present invention aims at developing a nucleic acid-based test or kit to detect and identify clinically important bacterial and fungal species responsible for invasive infections such as sepsis.

### DETAILED DESCRIPTION OF THE INVENTION

The present invention relates to a method of detecting a pathogen which may comprise exposing a sample containing or suspected of containing a pathogen with oligonucleotide mixtures comprising multiple oligonucleotide species, where each oligonucleotide species may be capable of specific binding with a genetic material of a pathogen selected from the group consisting of those of Table 4. In accordance with the present invention each of the oligonucleotide mixtures may be capable of amplifying the genetic material under similar or uniform amplification conditions and/or may be capable of hybridizing to the genetic material under similar or uniform hybridization conditions.

By carrying out the method of the present invention, the pathogen(s) present in a test sample, may thus be suitably identified.

In a particular embodiment of the invention, the multiple oligonucleotide species may comprise multiple sets of primer pairs which may be capable of specific amplification of the genetic material and the method may be carried out by exposing the sample with the multiple sets of primer pairs under conditions suitable for nucleic acid amplification.

In another particular embodiment of the invention, the multiple oligonucleotide species may comprise probes. In accordance with the present invention, each probe may be capable of hybridizing with the genetic material of one or more pathogen species. The sample may be exposed with the probe under conditions suitable for hybridization.

In an embodiment of the invention, the sample may be submitted to amplification using oligonucleotide species specific for the genetic material of each pathogen.

In another embodiment of the invention, the amplification step may be performed in separate vials or containers.

In a further embodiment, the amplification of the genetic material of each pathogen may be performed simultaneously.

In accordance with the present invention, the genetic material may be RNA or DNA.

It is well known in the art that RNA can be converted into DNA by the reverse transcriptase (RT) enzyme. Alternatively, DNA can be converted into RNA when, for example, an appropriate promoter (e.g. RNA polymerase promoter) and/or other regulatory elements are in operative connection with it. Therefore, the nucleic acid template (target) used to carry out the present invention may be either DNA (e.g., a genomic fragment or a restriction fragment) or RNA, either single-stranded or double-stranded.

The nucleic acid target (genome, gene or gene fragment (e.g., a restriction fragment) of the pathogen) may be in a purified, unpurified form or in an isolated form. The nucleic acid target may be contained within a sample including for example, a biological specimen obtained from a patient, a sample obtained from the environment (soil, objects, etc.), a microbial or tissue culture, a cell line, a preparation of pure or substantially pure pathogens or pathogen mixture etc. In accordance with the present invention, the sample may be obtained from patient having or suspected of having an infection.

The nucleic acid template may also be obtained from a biological or environmental sample, such as for example a specimen from a patient suspected of having an infection or carrying a pathogen, a food or animal specimen, a soil or water specimen, etc. The template may be a genetic material originating from the pathogen described herein including the complete genome, transcript, amplification product, fragments, etc. In an embodiment, the fragment may be of 50 to 1000 bases or base pairs or of 100 to 1000 bases or base pairs more and may encompass the region of hybridization of the nucleic acids of Table 1. Of course the length of the fragment may vary and encompass any sub-combinations found between 50 and 1000 bases or base pairs.

For each target gene, multiple sequence alignments have been generated using sequence data from evolutionary conserved protein-coding gene sequences database generated as described in international patent application NO. PCT/CA00/01150. Based on this analysis, conserved genetic regions were used to design broad-range primers useful for amplification of all representative strains of each targeted microbial species, complex or genus. In some cases, primers with a narrower range were also included to ensure efficient amplification for all target species. Primer pairs for the amplification of each target species have been chosen in order to be useful for the specific, sensitive, and ubiquitous amplification of all or most members within each target species, complex or genus (Table 1). For bacterial species, the tuf gene was the principal target and the recA gene was also used to facilitate the identification of some streptococcal species. For fungal species, the target was the tef1 gene encoding the eukaryotic elongation factor EF1-Alpha.

Aspects of the invention thus relate to individual primers, primer pairs or combination of primers or primer pairs for used in the methods and kits of the present invention.

Exemplary embodiments of individual primers, primer pairs and primer combinations are found below.

The present invention provides in a first embodiment, a nucleic acid which may comprise from 0 to 5 nucleotide addition or deletion at a 5′ end of SEQ ID NO.: 1.

In another embodiment, the present invention provides nucleic acid which may comprise from 0 to 5 nucleotide addition or deletion at a 5′ end of SEQ ID NO.: 2.

In a further embodiment, the present invention provides a nucleic acid which may comprise from 0 to 5 nucleotide addition or deletion at a 5′ end of SEQ ID NO.: 3.

In yet a further embodiment, the present invention provides a nucleic acid which may comprise from 0 to 5 nucleotide addition or deletion at a 5′ end of SEQ ID NO.: 4.

In an additional embodiment, the present invention provides a nucleic acid which may comprise from 0 to 5 nucleotides addition or deletion at a 5′ end of SEQ ID NO.: 5.

In yet an additional embodiment, the present invention provides a nucleic acid which may comprise from 0 to 5 nucleotide addition or deletion at a 5′ end of SEQ ID NO.: 6.

In another exemplary embodiment, the present invention provides a nucleic acid which may comprise from 0 to 5 nucleotide addition or deletion at a 5′ end of SEQ ID NO.: 7.

In yet another exemplary embodiment, the present invention provides a nucleic acid which may comprise from 0 to 5 nucleotide addition or deletion at a 5′ end of SEQ ID NO.: 8.

In still another embodiment, the present invention provides a nucleic acid which may comprise from 0 to 5 nucleotide addition or deletion at a 5′ end of SEQ ID NO.: 9.

In an additional embodiment, the present invention provides a nucleic acid which may comprise from 0 to 5 nucleotide addition or deletion at a 5′ end of SEQ ID NO.: 10.

In still another embodiment, the present invention provides a nucleic acid which may comprise from 0 to 5 nucleotide addition or deletion at a 5′ end of SEQ ID NO.: 11.

An additional embodiment of the present invention relates to a nucleic acid which may comprise from 0 to 5 nucleotide addition or deletion at a 5′ end of SEQ ID NO.: 12.

Yet an additional exemplary embodiment of the present invention provides a nucleic acid which may comprise from 0 to 5 nucleotide addition or deletion at a 5′ end of SEQ ID NO.: 13.

A further embodiment of the invention relates to a nucleic acid which may comprise from 0 to 5 nucleotide addition or deletion at a 5′ end of SEQ ID NO.: 14.

Another embodiment of the invention relates to a nucleic acid which may comprise from 0 to 5 nucleotide addition or deletion at a 5′ end of SEQ ID NO.: 15.

Yet another embodiment of the invention relates to a nucleic acid which may comprise from 0 to 5 nucleotide addition or deletion at a 5′ end of SEQ ID NO.: 16.

An additional embodiment of the invention relates to a nucleic acid which may comprise from 0 to 5 nucleotide addition or deletion at a 5′ end of SEQ ID NO.: 17.

Still an additional embodiment of the invention relates to a nucleic acid which may comprise from 0 to 5 nucleotide addition or deletion at a 5′ end of SEQ ID NO.: 18.

In a further exemplary embodiment, the present invention provides a nucleic acid which may comprise from 0 to 5 nucleotide addition or deletion at a 5′ end of SEQ ID NO.: 19.

In yet a further exemplary embodiment, the present invention provides a nucleic acid which may comprise from 0 to 5 nucleotide addition or deletion at a 5′ end of SEQ ID NO.: 20.

In an additional exemplary embodiment, the present invention provides a nucleic acid which may comprise from 0 to 5 nucleotide addition or deletion at a 5′ end of SEQ ID NO.: 21.

In yet an additional exemplary embodiment, the present invention provides a nucleic acid which may comprise from 0 to 5 nucleotide addition or deletion at a 5′ end of SEQ ID NO.: 22.

Another embodiment of the invention relates to a nucleic acid which may comprise from 0 to 5 nucleotide addition or deletion at a 5′ end of SEQ ID NO.: 23.

Still other embodiment of the invention relates to and a nucleic acid which may comprise from 0 to 5 nucleotide addition or deletion at a 5′ end of SEQ ID NO.: 24.

A further embodiment of the invention relates to a nucleic acid which may comprise from 0 to 5 nucleotide addition or deletion at a 5′ end of SEQ ID NO.: 25.

Still a further embodiment of the invention relates to a nucleic acid which may comprise from 0 to 5 nucleotide addition or deletion at a 5′ end of SEQ ID NO.: 375.

Another embodiment of the invention relates to a nucleic acid which may comprise from 0 to 5 nucleotide addition or deletion at a 5′ end of SEQ ID NO.: 376.

In an additional embodiment of the invention relates to a nucleic acid which may comprise from 0 to 5 nucleotide addition or deletion at a 5′ end of SEQ ID NO.: 377.

In yet an additional embodiment of the invention relates to a nucleic acid which may comprise from 0 to 5 nucleotide addition or deletion at a 5′ end of SEQ ID NO.: 378.

The invention also relates to primer pairs which may comprise at least two of the nucleic acids described above.

The invention therefore relates to primer pairs. Each set of primers may comprise at least one primer capable of specific amplification of the genetic material. The tested sample may thus be exposed with the multiple sets of primer pairs under conditions suitable for nucleic acid amplification.

Exemplary embodiments of primer pairs include the following.

A primer pair comprising a nucleic acid which may comprise from 0 to 5 nucleotide addition or deletion at a 5′ end of SEQ ID NO.: 1 and a nucleic acid which may comprise from 0 to 5 nucleotide addition or deletion at a 5′ end of SEQ ID NO.: 2.

A primer pair comprising a nucleic acid which may comprise from 0 to 5 nucleotides addition or deletion at a 5′ end of SEQ ID NO.: 3 and a nucleic acid which may comprise from 0 to 5 nucleotide addition or deletion at a 5′ end of SEQ ID NO.: 4.

A primer pair comprising a nucleic acid which may comprise from 0 to 5 nucleotide addition or deletion at a 5′ end of SEQ ID NO.: 5 and a nucleic acid which may comprise from 0 to 5 nucleotide addition or deletion at a 5′ end of SEQ ID NO.: 6.

A primer pair comprising a nucleic acid which may comprise from 0 to 5 nucleotide addition or deletion at a 5′ end of SEQ ID NO.: 7 and a nucleic acid which may comprise from 0 to 5 nucleotide addition or deletion at a 5′ end of SEQ ID NO.: 8.

A primer pair comprising a nucleic acid which may comprise from 0 to 5 nucleotide addition or deletion at a 5′ end of SEQ ID NO.: 375 and a nucleic acid which may comprise from 0 to 5 nucleotide addition or deletion at a 5′ end of SEQ ID NO.: 376.

In accordance with the present invention, the above mixture of primer pairs may be used to amplify the pathogen listed in Table 4.

In an exemplary embodiment, the amplification step may be performed using a combination of primers to form a first amplification multiplex reaction targeting at least the following bacterial species: Acinetobacter baumannii, Acinetobacter Iwoffii, Aeromonas caviae, Aeromonas hydrophila, Bacillus cereus, Bacillus subtilis, Citrobacter braakii, Citrobacter freundii, Citrobacter koseri, Enterobacter aerogenes, Enterobacter cloacae, Enterobacter sakazakii, Enterococcus faecium, Gemella haemolysans, Gemella morbillorum, Haemophilus influenzae, Kingella kingae, Klebsiella oxytoca, Klebsiella pneumoniae, Morganella morganii, Neisseria gonorrhoeae, Neisseria meningitidis, Pasteurella multocida, Propionibacterium acnes, Proteus mirabilis, Providencia rettgeri, Pseudomonas aeruginosa, Salmonella choleraesuis, Serratia liquefaciens, Serratia marcescens, Streptococcus agalactiae, Streptococcus anginosus, Streptococcus bovis, Streptococcus mutans, Streptococcus salivarius, Streptococcus sanguinis, Streptococcus suis, Vibrio vulnificus, Yersinia enterocolitica, Yersinia pestis/Yersinia pseudotuberculosis, Enterococcus faecalis, Clostridium perfringens, Corynebacterium jeikeium, and Capnocytophaga canimorsus.

This combination of primers may comprise:
a) a nucleic acid comprising from 0 to 5 nucleotide addition or deletion at a 5′ end of SEQ ID NO: 1,
b) a nucleic acid comprising from 0 to 5 nucleotide addition or deletion at a 5′ end of SEQ ID NO: 2,
c) a nucleic acid comprising from 0 to 5 nucleotide addition or deletion at a 5′ end of SEQ ID NO: 3,
d) a nucleic acid comprising from 0 to 5 nucleotide addition or deletion at a 5′ end of SEQ ID NO: 4,
e) a nucleic acid comprising from 0 to 5 nucleotide addition or deletion at a 5′ end of SEQ ID NO: 5,
f) a nucleic acid comprising from 0 to 5 nucleotide addition or deletion at a 5′ end of SEQ ID NO: 6,
g) a nucleic acid comprising from 0 to 5 nucleotide addition or deletion at a 5′ end of SEQ ID NO: 7,
h) a nucleic acid comprising from 0 to 5 nucleotide addition or deletion at a 5′ end of SEQ ID NO: 8,
i) a nucleic acid comprising from 0 to 5 nucleotide addition or deletion at a 5′ end of SEQ ID NO: 375, and;
j) a nucleic acid comprising from 0 to 5 nucleotide addition or deletion at a 5′ end of SEQ ID NO: 376.

In a more specific embodiment the combination of primers used in the first multiplex reaction includes SEQ ID NO: 375 and SEQ ID NO: 376 (identified as SEQ ID NOs: 636 and 637 respectively in international patent application NO. PCT/CA00/01150) with primers SEQ ID NOs: 1 to 8.

Other exemplary embodiments of primer pairs include the following.

A primer pair comprising a nucleic acid which may comprise from 0 to 5 nucleotide addition or deletion at a 5′ end of SEQ ID NO.: 9 and a nucleic acid which may comprise from 0 to 5 nucleotide addition or deletion at a 5′ end of SEQ ID NO.: 10.

A primer pair comprising a nucleic acid which may comprise from 0 to 5 nucleotides addition or deletion at a 5′ end of SEQ ID NO.: 11 and a nucleic acid which may comprise from 0 to 5 nucleotide addition or deletion at a 5′ end of SEQ ID NO.: 12.

A primer pair comprising a nucleic acid which may comprise from 0 to 5 nucleotide addition or deletion at a 5′ end of SEQ ID NO.: 13 and a nucleic acid which may comprise from 0 to 5 nucleotide addition or deletion at a 5′ end of SEQ ID NO.: 14.

In accordance with the present invention, the above mixture of primer pairs may be used to amplify the pathogen listed in Table 4.

In an exemplary embodiment, the amplification step may be performed using a combination of primers to form a second amplification multiplex reaction targeting at least the following bacterial species: Citrobacter freundii, Citrobacter koseri, Enterobacter aerogenes, Enterobacter cloacae, Enterobacter sakazakii, Klebsiella oxytoca, Klebsiella pneumoniae, Salmonella choleraesuis, Listeria monocytogenes, Pasteurella pneumotropica, Staphylococcus aureus, Staphylococcus epidermidis, Staphylococcus haemolyticus, Staphylococcus hominis, Staphylococcus saccharolyticus, Staphylococccus saprophyticus, Staphylococcus warneri, Streptococcus dysgalactiae, Streptococcus pneumoniae, and Streptococcus pyogenes.

This combination of primers may comprise:
a) a nucleic acid comprising from 0 to 5 nucleotide addition or deletion at a 5′ end of SEQ ID NO: 9,
b) a nucleic acid comprising from 0 to 5 nucleotide addition or deletion at a 5′ end of SEQ ID NO: 10,
c) a nucleic acid comprising from 0 to 5 nucleotide addition or deletion at a 5′ end of SEQ ID NO: 11,
d) a nucleic acid comprising from 0 to 5 nucleotide addition or deletion at a 5′ end of SEQ ID NO: 12,
e) a nucleic acid comprising from 0 to 5 nucleotide addition or deletion at a 5′ end of SEQ ID NO: 13, and;
f) a nucleic acid comprising from 0 to 5 nucleotide addition or deletion at a 5′ end of SEQ ID NO: 14.
   In a more specific embodiment the combination of primers used in the second multiplex reaction includes SEQ ID NOs: 9 to 14.

Yet other exemplary embodiments of primer pairs include the following.

A primer pair comprising a nucleic acid which may comprise from 0 to 5 nucleotide addition or deletion at a 5′ end of SEQ ID NO.: 15 and a nucleic acid which may comprise from 0 to 5 nucleotide addition or deletion at a 5′ end of SEQ ID NO.: 16.

A primer pair comprising a nucleic acid which may comprise from 0 to 5 nucleotides addition or deletion at a 5′ end of SEQ ID NO.: 15 and a nucleic acid which may comprise from 0 to 5 nucleotide addition or deletion at a 5′ end of SEQ ID NO.: 17.

A primer pair comprising a nucleic acid which may comprise from 0 to 5 nucleotide addition or deletion at a 5′ end of SEQ ID NO.: 18 and a nucleic acid which may comprise from 0 to 5 nucleotide addition or deletion at a 5′ end of SEQ ID NO.: 19.

A primer pair comprising a nucleic acid which may comprise from 0 to 5 nucleotide addition or deletion at a 5′ end of SEQ ID NO.: 18 and a nucleic acid which may comprise from 0 to 5 nucleotide addition or deletion at a 5′ end of SEQ ID NO.: 20.

A primer pair comprising a nucleic acid which may comprise from 0 to 5 nucleotide addition or deletion at a 5′ end of SEQ ID NO.: 18 and a nucleic acid which may comprise from 0 to 5 nucleotide addition or deletion at a 5′ end of SEQ ID NO.: 21.

In accordance with the present invention, the above mixture of primer pairs may be used to amplify the pathogen listed in Table 4.

An additional exemplary embodiment of the present invention relates to the combination of primers to form a third amplification multiplex reaction targeting at least the following fungal species: Candida albicans, Candida glabrata, Candida parapsilosis, Candida tropicalis, Candida krusei, Aspergillus fumigatus, Aspergillus niger, Aspergillus nidulans, Aspergillus flavus, and Aspergillus terreus.

This combination of primers may comprise:
a) a nucleic acid comprising from 0 to 5 nucleotide addition or deletion at a 5′ end of SEQ ID NO: 15,
b) a nucleic acid comprising from 0 to 5 nucleotide addition or deletion at a 5′ end of SEQ ID NO: 16,
c) a nucleic acid comprising from 0 to 5 nucleotide addition or deletion at a 5′ end of SEQ ID NO: 17,
d) a nucleic acid comprising from 0 to 5 nucleotide addition or deletion at a 5′ end of SEQ ID NO: 18,
e) a nucleic acid comprising from 0 to 5 nucleotide addition or deletion at a 5′ end of SEQ ID NO: 19,
f) a nucleic acid comprising from 0 to 5 nucleotide addition or deletion at a 5′ end of SEQ ID NO: 20, and;
g) a nucleic acid comprising from 0 to 5 nucleotide addition or deletion at a 5′ end of SEQ ID NO: 21.

In a more specific embodiment the combination of primers used to form the third amplification multiplex reaction includes SEQ ID NOs: 15 to 21.

Further exemplary embodiments of primer pairs include the following.

A primer pair comprising a nucleic acid which may comprise from 0 to 5 nucleotide addition or deletion at a 5′ end of SEQ ID NO.: 22 and a nucleic acid which may comprise from 0 to 5 nucleotide addition or deletion at a 5′ end of SEQ ID NO.: 23.

A primer pair comprising a nucleic acid which may comprise from 0 to 5 nucleotide addition or deletion at a 5′ end of SEQ ID NO.: 24 and a nucleic acid which may comprise from 0 to 5 nucleotide addition or deletion at a 5′ end of SEQ ID NO.: 25.

A primer pair comprising a nucleic acid which may comprise from 0 to 5 nucleotide addition or deletion at a 5′ end of SEQ ID NO.: 26 and a nucleic acid which may comprise from 0 to 5 nucleotide addition or deletion at a 5′ end of SEQ ID NO.: 23.

A primer pair comprising a nucleic acid which may comprise from 0 to 5 nucleotide addition or deletion at a 5′ end of SEQ ID NO.: 377 and a nucleic acid which may comprise from 0 to 5 nucleotide addition or deletion at a 5′ end of SEQ ID NO.: 378.

In accordance with the present invention, the above mixture of some of primer pairs may be used to amplify the pathogen listed in Table 4.

Another exemplary embodiment of the present invention relates to a combination of primers to form amplification multiplex reaction number four (version 1) targeting at least the following bacterial species: Bacteroides fragilis, Brucella melitensis, Burkholderia cepacia, Stenotrophomonas maltophilia, and Escherichia coli-Shigella sp.

This combination of primers may comprise:
a) a nucleic acid comprising from 0 to 5 nucleotide addition or deletion at a 5′ end of SEQ ID NO: 22,
b) a nucleic acid comprising from 0 to 5 nucleotide addition or deletion at a 5′ end of SEQ ID NO: 23,
c) a nucleic acid comprising from 0 to 5 nucleotide addition or deletion at a 5′ end of SEQ ID NO: 24,
d) a nucleic acid comprising from 0 to 5 nucleotide addition or deletion at a 5′ end of SEQ ID NO: 25,
e) a nucleic acid comprising from 0 to 5 nucleotide addition or deletion at a 5′ end of SEQ ID NO: 377, and;
f) a nucleic acid comprising from 0 to 5 nucleotide addition or deletion at a 5′ end of SEQ ID NO: 378.

In a more specific embodiment, the combination of primers SEQ ID NOs: 22 to 25 with primers SEQ ID NO: 377 and SEQ ID NO: 378 (identified as SEQ ID NOs: 1661 and 1665 respectively in international patent application NO. PCT/CA00/01150) are used to form amplification multiplex reaction number four (version 1).

Although, Streptomyces avermitilis is not considered a pathogenic species, primers for its amplification were also included in this multiplex for use as control purposes and as such, SEQ ID NO: 24 and 25 may be omitted. It is to be understood herein that controls are used to validate the assays and although useful, any of the controls or related reagents thereof are optional and/or may easily be omitted or replaced by other controls.

Another exemplary embodiment of the present invention relates to a combination of primers to form amplification multiplex reaction number four (version 2) targeting at least the following bacterial species: Bacteroides fragilis, Brucella melitensis, Burkholderia cepacia, Stenotrophomonas maltophilia, and Escherichia coli-Shigella sp.

This combination of primers may comprise:
a) a nucleic acid comprising from 0 to 5 nucleotide addition or deletion at a 5′ end of SEQ ID NO: 22,
b) a nucleic acid comprising from 0 to 5 nucleotide addition or deletion at a 5′ end of SEQ ID NO: 23, and;
c) a nucleic acid comprising from 0 to 5 nucleotide addition or deletion at a 5′ end of SEQ ID NO: 26.

In a more specific embodiment the combination of primers SEQ ID NOs: 22, 23 and 26 are used to form amplification multiplex reaction number four (version 2).

It is to be understood herein that distinction among each of the bacterial or fungal species may be achieved in different manners. In an embodiment of the invention, distinction of each species may be achieved with oligonucleotide probes specific for each species.

Other aspects of the invention therefore relates to oligonucleotide capture probe sequences. These oligonucleotides may be used for example, for solid support hybridization. An advantage of these probes is that may be used under uniform hybridization conditions (e.g., stringency) to specifically detect and identify the targeted microbial species.

Yet in another embodiment, a combination of a relatively small number of probe sequences are used for the identification of bacterial and fungal species.

For example, nucleic acid hybridization probes targeting internal regions of the PCR amplicons generated using the amplification primer combinations described herein are encompassed by the present invention. The group of PCR-generated nucleic acid templates is prepared from one or more of the target microbial species mentioned above. These hybridization probes can be used either for real-time PCR detection (e.g. TaqMan probes, molecular beacons) or for solid support hybridization (e.g. microarray hybridization, bead-based capture of nucleic acids).

Exemplary embodiments of probes include the following.

A nucleic acid which may comprise from 0 to 5 nucleotide addition, deletion or combination of addition and deletion at a 5′ end and/or 3′ end thereof of any one of the probes listed in Table 2 or a complement thereof. For purpose of concision the Applicant has not provided a complete list of each specific example of such nucleic acid but it is to be understood herein the language recited is to be applied for each nucleic acid sequences individually or collectively.

Exemplary embodiments of individual probes includes the following:

A nucleic acid which may comprise from 0 to 5 nucleotide addition, deletion or combination of addition and deletion at a 5′ end and/or 3′ end thereof of SEQ ID NO.: 27 or a complement thereof.

A nucleic acid which may comprise from 0 to 5 nucleotide addition, deletion or combination of addition and deletion at a 5′ end and/or 3′ end thereof of SEQ ID NO.: 28 or a complement thereof.

A nucleic acid which may comprise from 0 to 5 nucleotide addition, deletion or combination of addition and deletion at a 5′ end and/or 3′ end thereof of SEQ ID NO.: 29 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 1.

A further embodiment combines any or all probes SEQ ID NOs: 27 to 203 of the present invention to react with the amplification products of the first amplification multiplex reaction. An exemplary embodiment of a sub-combination or probes (without the control used herein) includes SEQ ID NOs: 27 to 125 and SEQ ID NOs: 131 to 203.

A more specific embodiment combines the selected set of probes SEQ ID NOs: 27 to 44, 46 to 63, 65 to 71, 73 to 77, 79 to 97, 99 to 125, 127, 129, 131 to 203 of the present invention to react with the amplification products of amplification multiplex reaction number one. An exemplary embodiment of a sub-combination of probes (without the control used herein) includes SEQ ID NOs: 27 to 44, 46 to 63, 65 to 71, 73 to 77, 79 to 97, 99 to 125, and 131 to 203.

Other exemplary embodiments of individual probes include the following:

A nucleic acid which may comprise from 0 to 5 nucleotide addition, deletion or combination of addition and deletion at a 5′ end and/or 3′ end thereof of SEQ ID NO.: 204 or a complement thereof.

A nucleic acid which may comprise from 0 to 5 nucleotide addition, deletion or combination of addition and deletion at a 5′ end and/or 3′ end thereof of SEQ ID NO.: 205 or a complement thereof.

A nucleic acid which may comprise from 0 to 5 nucleotide addition, deletion or combination of addition and deletion at a 5′ end and/or 3′ end thereof of SEQ ID NO.: 206 or a complement thereof.

A nucleic acid which may comprise from 0 to 5 nucleotide addition, deletion or combination of addition and deletion at a 5′ end and/or 3′ end thereof of SEQ ID NO.: 207 or a complement thereof.

A nucleic acid which may comprise from 0 to 5 nucleotide addition, deletion or combination of addition and deletion at a 5′ end and/or 3′ end thereof of SEQ ID NO.: 208 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 2.

A further embodiment combines any or all probes SEQ ID NOs: 204 to 293, 364 and 365 of the present invention to react with the amplification products of the second amplification multiplex reaction. An exemplary embodiment of a sub-combination or probes (without the control used herein) includes SEQ ID NOs: 204 to 237, SEQ ID NOs: 241 to 293 and SEQ ID NO: 364.

A specific embodiment combines the selected set of probes SEQ ID NOs: 204, 208, 211, 212, 214, 215, 219, 223, 226, 227, 229, 231, 233, 236, 241, 242, 244, 246, 248, 249, 253 to 256, 261, 264 to 267, 270, 272, 279 to 281, 284 to 288, 291, 292, 364, and 365 of the present invention to react with the amplification products of amplification multiplex reaction number two. An exemplary embodiment of a sub-combination of probes (without the control used herein) includes SEQ ID NOs: 204, 208, 211, 212, 214, 215, 219, 223, 226, 227, 229, 231, 233, 236, 241, 242, 244, 246, 248, 249, 253 to 256, 261, 264 to 267, 270, 272, 279 to 281, 284 to 288, 291, 292 and 364.

Yet other exemplary embodiments of individual probes include the following:

A nucleic acid which may comprise from 0 to 5 nucleotide addition, deletion or combination of addition and deletion at a 5′ end and/or 3′ end thereof of SEQ ID NO.: 294 or a complement thereof.

A nucleic acid which may comprise from 0 to 5 nucleotide addition, deletion or combination of addition and deletion at a 5′ end and/or 3′ end thereof of SEQ ID NO.: 295 or a complement thereof.

A nucleic acid which may comprise from 0 to 5 nucleotide addition, deletion or combination of addition and deletion at a 5′ end and/or 3′ end thereof of SEQ ID NO.: 296 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 3.

A further embodiment combines any or all probes SEQ ID NOs: 294 to 338 of the present invention to react with the amplification products of the third amplification multiplex reaction. An exemplary embodiment of a sub-combination or probes (without the control used herein) includes SEQ ID NOs: 294 to 333.

Yet a further specific embodiment combines the selected set of probes SEQ ID NOs: 294, 296 to 309, 312, 314, 316, 317, 318, 320 to 323, 326 to 330, 332, and 335 of the present invention to react with the amplification products of amplification multiplex reaction number three. An exemplary embodiment of a sub-combination of probes (without the control used herein) includes SEQ ID NOs: 294, 296 to 309, 312, 314, 316, 317, 318, 320 to 323, 326 to 330 and 332.

Additional exemplary embodiments of individual probes include the following:

A nucleic acid which may comprise from 0 to 5 nucleotide addition, deletion or combination of addition and deletion at a 5′ end and/or 3′ end thereof of SEQ ID NO.: 339 or a complement thereof.

A nucleic acid which may comprise from 0 to 5 nucleotide addition, deletion or combination of addition and deletion at a 5′ end and/or 3′ end thereof of SEQ ID NO.: 340 or a complement thereof.

A nucleic acid which may comprise from 0 to 5 nucleotide addition, deletion or combination of addition and deletion at a 5′ end and/or 3′ end thereof of SEQ ID NO.: 341 or a complement thereof.

Other specific embodiment of individual probes relates to individual nucleic acids which may comprise from 0 to 5 nucleotide addition, deletion or combination of addition and deletion at a 5′ end and/or 3′ end thereof to any of those listed in Table 2 and identified for Multiplex 4.

A further embodiment combines any or all probes SEQ ID NOs: 339 to 363 and 366 to 374 of the present invention to react with the amplification products of the fourth amplification multiplex reaction. An exemplary embodiment of a sub-combination or probes (without the control used herein) includes SEQ ID NOs: 339 to 352, SEQ ID NO: 356, SEQ ID NO: 357 and SEQ ID NOs: 366 to 374.

Another specific embodiment combines the selected set of probes SEQ ID NOs: 339 to 344, 348, 353 and 366 to 374 of the present invention to react with the amplification products of amplification multiplex reaction number four. An exemplary embodiment of a sub-combination of probes (without the control used herein) includes SEQ ID NOs: 339 to 344, 348 and 366 to 374.

In another embodiment probes SEQ ID NOs: 27 to 374 of the present invention are used to react with the amplification products of any of the four amplification multiplex reactions described above.

The combination of the following probes were found to be particularly useful for detection purposes.

A nucleic acid which may comprise from 0 to 5 nucleotide addition, deletion or combination of addition and deletion at a 5′ end and/or 3′ end thereof to any of SEQ ID NOs: 27 to 44, 46 to 63, 65 to 71, 73 to 77, 79 to 97, 99 to 125, 127, 129, 131 to 203 or a complement thereof. As indicated herein, the control probes SEQ ID NO: 127 and/or 129 may be replaced or omitted.

A nucleic acid which may comprise from 0 to 5 nucleotide addition, deletion or combination of addition and deletion at a 5′ end and/or 3′ end thereof to any of SEQ ID NOs: 204, 208, 211, 212, 214, 215, 219, 223, 226, 227, 229, 231, 233, 236, 241, 242, 244, 246, 248, 249, 253 to 256, 261, 264 to 267, 270, 272, 279 to 281, 284 to 288, 291, 292, 364, and 365 or a complement thereof. As indicated herein, the control probe SEQ ID NO: 365 may be replaced or omitted.

A nucleic acid which may comprise from 0 to 5 nucleotide addition, deletion or combination of addition and deletion at a 5′ end and/or 3′ end thereof to any of SEQ ID NOs: 294, 296 to 309, 312, 314, 316, 317, 318, 320 to 323, 326 to 330, 332, and 335 or a complement thereof. As indicated herein, the control probe SEQ ID NO: 335 may be replaced or omitted.

A nucleic acid which may comprise from 0 to 5 nucleotide addition, deletion or combination of addition and deletion at a 5′ end and/or 3′ end thereof to any of SEQ ID NOs: 339 to 344, 348, 353 and 366 to 374 or a complement thereof. As indicated herein, the control probe SEQ ID NO: 353 may be replaced or omitted.

The present invention also covers detection of amplification products by hybridization with specific probes anchored onto a solid support (e.g. microarray hybridization). A specific amplification product can be formed when a test sample contains the target microbial nucleic acid. Upon amplification, a fluorescent dye (e.g., Cy-3) is incorporated into the amplicon, and detected with a fluorescence scanner. Oligonucleotide probes sequences were selected using multiple sequence alignments to identify sequences or sequence combinations unique to each bacterial and fungal species, complex or genus. To cover all or most strains of a target species or genus, several probes have been designed for the ubiquitous species-specific/genus-specific detection of the target bacterial or fungal nucleic acid sequence. In some cases, a single amplicon per species was not sufficient for proper identification. This is why for some species, more than one amplicon was used for correct identification. Loy and Bodrossy recently reviewed conditions required to obtain probe set combinations presenting the essential characteristics of specificity, sensitivity and uniformity (Loy, A. and Bodrossy, L., 2006, Clin. Chim. Acta 363:106-119). They state that the ideal properties of highly specific recognition, efficient binding and uniform thermodynamic behaviour represent conflicting goals difficult to achieve in practice. They propose to use careful design rules but they admit that the predictive value of these rules is known to be unreliable for solid support hybridization and experimental validation of the probe combinations is required. Another approach they suggest is to add redundancy in the probe combination strategy. However, adding more probes increases cost and complexity while limiting miniaturization and parallelization capacity. It is an object of the present invention to provide an optimal set of probe sequences capable of reaching the goals of specificity, sensitivity and uniformity under common hybridization conditions on solid support for the detection and identification of invasive bacterial and fungal species.

The present invention features hybridization probes chosen from the regions amplified with the PCR primer pairs described above. Probes selected for the optimal multiplex assays are listed in Table 2. However, in some embodiments one probe per target amplicon may be sufficient to detect a pathogen of interest. For example, among the probes of Table 2 used to detect Acinetobacter baumannii, an assay using only one, two, three or four probes among SEQ ID NOs: 27, 28, 29, 30 or 31 may still function. The same may also be found true for each of the pathogen listed in Table 2. Therefore, detection of the pathogens of Table 4 may be carried out with all the sepsis-associated pathogen probes of Table 2 or with subselections comprising 1, 2, 3, 4, 5, 6, 7, 8, 9 or 10 pathogen-specific probes of Table 2. As used herein the term “pathogen-specific probe” includes one or more probes which are used to detect a given pathogen. Of course additional pathogen-specific probes other than those listed in Table 2 may be used to detect the pathogen listed in Table 4.

In yet another aspect of this invention, amplification primers are labelled with a fluorophore such as Cy-3 and the generated amplicons are detected by hybridization with genus-, group (sometimes referred to as multispecies complex)- or species-specific capture probes.

As part of the design strategy, all oligonucleotides probes for hybridization and primers for DNA amplification by PCR were evaluated for their suitability for hybridization or PCR amplification by computer analysis using commercially available programs such as the Wisconsin Genetics Computer Group (GCG) program package, and the primer analysis software Oligo™ 6.7 (Molecular Biology Insights inc.). The potential suitability of the PCR primer pairs was also evaluated prior to synthesis by verifying the absence of unwanted features such potential to form dimers or internal secondary structure, or having long stretches of one nucleotide and a high proportion of guanine or cytosine residues at the 3′ end. Multiplexing PCR primers represents a challenge since the presence of several pairs of primers together in the same tube increases chances of mispairing and formation of unwanted non-specific amplification products such as primer dimers.

Nucleotide bases single letter codes have been used herein in accordance with the International Union of Biochemistry (IUB) are A: Adenine, C: Cytosine, G: Guanine, T: Thymine, U: Uridine, and I: Inosine. For sequence degeneracies the IUB codes are M: Adenine or Cytosine, R: Adenine or Guanine, W: Adenine or Thymine, S: Cytosine or Guanine, Y: Cytosine or Thymine, and K: Guanine or Thymine.

| Bases | Code |
|---|---|
| A or C | M |
| A or G | R |
| A or T | W |
| C or G | S |
| C or T | Y |
| G or T | K |
| Inosine | I |

Several primers have been designed to efficiently amplify the pathogens described herein. It is to be understood that each of the oligonucleotides individually possess their own utility as it may be possible to use such oligonucleotides for other purposes than those described herein. For example, primers of the present invention may be combined with other primers for amplification of a longer or shorter amplicon. Probes of the present invention may be combined with other probes in detection tools such as microarrays.

The oligonucleotide sequence of primers or probes may be derived from either strand of the duplex DNA. The primers or probes may consist of the bases A, G, C, or T or analogs and they may be degenerated at one or more chosen nucleotide position(s) to ensure DNA amplification for all strains of a target bacterial or fungal species. Degenerated primers are primers which have a number of possibilities at mismatch positions in the sequence in order to allow annealing to complementary sequences and amplification of a variety of related sequences. For example, the following primer AYATTAGTGCTTTTAAAGCC is an equimolar mix of the primers ACATTAGTGCTTTTAAAGCC and ATATTAGTGCTTTTAAAGCC. Degeneracies obviously reduce the specificity of the primer(s), meaning mismatch opportunities are greater, and background noise increases; also, increased degeneracy means concentration of the individual primers decreases; hence, greater than 512-fold degeneracy is preferably avoided. Thus, degenerated primers should be carefully designed in order to avoid affecting the sensitivity and/or specificity of the assay. Inosine is a modified base that can bind with any of the regular base (A, T, C or G). Inosine is used in order to minimize the number of degeneracies in an oligonucleotide.

The present invention also features hybridization probes chosen from the regions amplified with the PCR primer pairs described above, i.e., binding within the PCR amplicon amplified by the primers listed in Table 1. Exemplary embodiments of probes selected for the optimal multiplex assays are listed in Table 2. These probes can be used for detecting the selected pathogens by either hybridizing to target pathogen nucleic acids amplified with the selected primer pairs or to unamplified target pathogens nucleic acids using signal amplification methods such as ultra-sensitive biosensors. When a probe is combined with other probes for simultaneous detection of multiple pathogens, the specificity of the probe should not be substantially affected by the presence of other probes, i.e., it still hybridizes to the target pathogens nucleic acid. Preferably, a probe selected for one pathogen does not hybridize to a nucleic acid from another pathogen.

The primers or probes may be of any suitable length determined by the user. In an embodiment of the present invention, the primers and/or probes (independently from one another) may be for example, from 10 to 50 nucleotide long (inclusively), from 10 to 40, from 10 to 35, from 10 to 30, from 12 to 40, from 12 to 25 nucleotide long (inclusively), from 15 to 25 nucleotide long (inclusively), from 15 to 20 nucleotides long (inclusively), etc. Although for purpose of concision, the complete list of combination of length between 10 to 50 nucleotides long is not provided herein it is intended that each and every possible combinations that may be found between 10 to 50 nucleotides (inclusively) be covered. A few examples only of such possible combination is provided as follow, 10 to 30, 11 to 30, 10 to 29, 11 to 29, 15 to 17, 14 to 21, etc.

For the primer sequences listed in Table 1, variant sequences comprising short (up to 20% of the total length of the oligonucleotide) extension or reduction of the sequence on the 5′ side are also an object of this invention. In accordance with an embodiment of the invention the primer may thus comprise an addition of 1 to 5 nucleotides at the 5′ end thereof. Also in accordance with an embodiment of the invention the primer may comprise a deletion of 1 to 5 nucleotides at the 5′ end thereof.

For the probe sequences listed in Table 2, variant sequences comprising short (20%) extension, reduction and/or displacement of the sequence on the 5′ and/or the 3′ side compared to the target gene fragment are also an object of this invention. In accordance with an embodiment of the invention the probe may thus comprise an addition of 1 to 5 nucleotides at the 5′ end thereof. In accordance with another embodiment of the invention the probe may thus comprise an addition of 1 to 5 nucleotides at the 3′ end thereof. Also in accordance with an embodiment of the invention the probe may comprise a deletion of 1 to 5 nucleotides at the 5′ end thereof. Further in accordance with an embodiment of the invention the probe may comprise a deletion of 1 to 5 nucleotides at the 3′ end thereof.

As used herein the term “at least two” encompasses, “at least three”, “at least four”, “at least five”, “at least six”, “at least seven”, “at least eight”, “at least nine”, “at least ten”, “at least eleven”, “at least twelve”, “at least thirteen”, “at least fourteen”, “at least fifteen”, “at least sixteen”, “at least seventeen”, “at least eighteen”, “at least nineteen”, “at least twenty”, “at least twenty-one”, “at least twenty-two”, “at least twenty-three”, “at least twenty-four”, “at least twenty-five”, “at least twenty-six”, “at least twenty-seven”, “at least twenty-eight”, etc.

In another embodiment of the invention, the primers and/or probe (independently from one another) may be at least 10 nucleotides long, at least 11 nucleotides long, at least 12 nucleotides long, at least 13 nucleotides long, at least 14 nucleotides long, at least 15 nucleotides long, at least 16 nucleotides long, at least 17 nucleotides long, at least 18 nucleotides long, at least 19 nucleotides long, at least 20 nucleotides long, at least 21 nucleotides long, at least 22 nucleotides long, at least 23 nucleotides long, at least 24 nucleotides long, at least 25 nucleotides long, at least 26 nucleotides long, etc.

The primers and/or probes described in Table 1 and Table 2 may thus comprise additional nucleotides at their 5′ end and/or 3′ end. The identity of these nucleotides may vary. In some instances, the nucleotide may be chosen among the conventional A, T, G, or C bases while in other instances, the nucleotide may be a modified nucleotide as known in the art. However, in an embodiment of the invention, the additional nucleotide may correspond to the nucleotide found in any of the corresponding gene sequence found in public databases.

As used herein the term “comprising from 0 to 5 additional nucleotides at a 5′ end and/or 3′ end thereof” means that the oligonucleotide or nucleic acid may have either, a) 0, 1, 2, 3, 4 or 5 additional nucleotide at its 5′ end, b) 0, 1, 2, 3, 4 or 5 additional nucleotide at its 3′ end or c) 0, 1, 2, 3, 4 or 5 additional nucleotide at its 5′ end and 0, 1, 2, 3, 4 or 5 additional nucleotide at its 3′ end.

As used herein the term “comprising from 0 to 5 nucleotides deletion at a 5′ end and/or 3′ end thereof” means that the oligonucleotide or nucleic acid may have either, a) 0, 1, 2, 3, 4 or 5 nucleotide deleted at its 5′ end, b) 0, 1, 2, 3, 4 or 5 nucleotide deleted at its 3′ end or c) 0, 1, 2, 3, 4 or 5 nucleotide deleted at its 5′ end and 0, 1, 2, 3, 4 or 5 nucleotide deleted at its 3′ end.

As used herein the term “comprising from 0 to 5 additional nucleotides at one of a 5′ end or 3′ end and/or a deletion of from 0 to 5 nucleotides at the other of a 5′ end or 3′ end” means that the oligonucleotide or nucleic acid may have either, a) 0, 1, 2, 3, 4 or 5 additional nucleotide at its 5′ end and 0, 1, 2, 3, 4 or 5 nucleotides deleted at its 3′ end or b) 0, 1, 2, 3, 4 or 5 additional nucleotide at its 3′ end and 0, 1, 2, 3, 4 or 5 nucleotides deleted at its 5′ end, c) 0, 1, 2, 3, 4 or 5 additional nucleotide at its 5′ end and 0, 1, 2, 3, 4 or 5 additional nucleotides at its 3′ end or d) 0, 1, 2, 3, 4 or 5 nucleotide deleted at its 5′ end and 0, 1, 2, 3, 4 or 5 nucleotides deleted at its 3′ end.

The term “comprising from 0 to 5” also encompasses “comprising from 1 to 5”, “comprising from 2 to 5”, “comprising from 3 to 5”; “comprising from 4 to 5”, “comprising from 0 to 4”, “comprising from 1 to 4”; “comprising from 2 to 4”, “comprising from 3 to 4”, “comprising from 0 to 3” “comprising from 1 to 3”; “comprising from 2 to 3”, “comprising from 0 to 2”, “comprising from 0 to 1”, “comprising 0”, “comprising 1”, “comprising 2”, “comprising 3”, “comprising 4”, or “comprising 5”.

As used herein the term “complement” with respect to nucleic acid molecules refers to a molecule that is able of base pairing with another nucleic acid molecule with for example a perfect (e.g., 100%) match over a portion thereof.

In accordance with the present invention, the primers and/or probes may be labelled. In an embodiment of the invention, the primers may be labelled with a fluorophore therefore providing a labelled target amplicon. In another embodiment, the probes may be labelled with a fluorophore.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³2P), phosphorescent labels, enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, each of which is hereby incorporated by reference in its entirety for all purposes. Fluorescent labels may easily be added during an in vitro transcription reaction and thus represent an interesting avenue.

In addition to the specific oligonucleotides mentioned herein, the methods and kits may further comprise controls, such as control primers, control probes, control samples, etc. Although exemplary embodiments of controls have been provided in herein, a person of skill in the art will understand that any type of controls may be used to validate the methods.

As illustrated in Table 3, a significant proportion of designed primer and probe sequences were not retained for the final multiplex combinations due to their poor performance during the experimental validation procedure. Only those listed in Table 1 or Table 2 have been retained.

It is to be understood herein that the separation of the amplification reactions into four multiplexes has been found to conveniently work. However, the amplification may be separated into more than four reactions. For example, although less convenient, each of the multiplex 1, 2, 3 or 4 could be subdivided in 2, 3 or 4 distinct amplification reactions where relevant for a total of up to 16 reactions.

One method which is currently used for amplifying genetic material is the polymerase chain reaction (PCR) or the reverse transcriptase polymerase chain reaction (RT-PCR). However, in some instances, the nucleic acids may be in a sufficient amount that amplification is not required.

As the method was designed to use similar experimental conditions, the PCR amplification for each multiplex can be performed using the same thermal cycling profile thereby allowing the amplification of all the nucleic acid targets at the same time in a single apparatus (e.g., thermocycler).

Although nucleic acid amplification is often performed by PCR or RT-PCR, other methods exist. Non-limiting examples of such method include quantitative polymerase chain reaction (Q-PCR), ligase chain reaction (LCR), transcription-mediated amplification (TMA), self-sustained sequence replication (3SR), nucleic acid sequence-based amplification (NASBA), strand displacement amplification (SDA), recombinase polymerase amplification (RPA), loop-mediated isothermal amplification (LAMP), helicase-dependent amplification (HDA), helicase-dependent isothermal DNA amplification (tHDA), branched DNA (bDNA), cycling probe technology (CPT), solid phase amplification (SPA), rolling circle amplification technology (RCA), real-time RCA, solid phase RCA, RCA coupled with molecular padlock probe (MPP/RCA), aptamer based RCA (aptamer-RCA), anchored SDA, primer extension preamplification (PEP), degenerate oligonucleotide primed PCR (DOP-PCR), sequence-independent single primer amplification (SISPA), linker-adaptor PCR, nuclease dependent signal amplification (NDSA), ramification amplification (RAM), multiple displacement amplification (MDA), real-time RAM, and whole genome amplification (WGA) (Westin, L. et al., 2000, Nat. Biotechnol. 18:199-204; Notomi, T. et al., 2000, Nucleic Acids Res. 28:e63; Vincent, M. et al., 2004, EMBO reports 5:795-800; Piepenburg, O. et al., 2006, PLoS Biology 4:E204; Yi, J. et al., 2006, Nucleic Acids Res. 34:e81; Zhang, D. et al., 2006, Clin. Chim. Acta 363:61-70; McCarthy, E. L. et al., 2007, Biosens. Biotechnol. 22:126-1244; Zhou, L. et al., 2007, Anal. Chem. 79:7492-7500; Coskun, S. and Alsmadi, O., 2007, Prenat. Diagn. 27:297-302; Biagini, P. et al., 2007, J. Gen. Virol. 88:2629-2701; Gill, P. et al., 2007, Diagn. Microbiol. Infect. Dis. 59:243-249; Lasken, R. S. and Egholm, M., 2003, Trends Biotech. 21:531-535).

The scope of this invention is not limited to the use of amplification by PCR technologies, but rather includes the use of any nucleic acid amplification method or any other procedure which may be used to increase the sensitivity and/or the rapidity of nucleic acid-based diagnostic tests. The scope of the present invention also covers the use of any nucleic acid amplification and detection technology including real-time or post-amplification detection technologies, any amplification technology combined with detection, any hybridization nucleic acid chips or array technologies, any amplification chips or combination of amplification and microarray hybridization technologies. Amplification and/or detection using a microfluidic system or a micro total analysis system (μTAS) is under the scope of this invention. Detection and identification by any nucleic acid sequencing method is also under the scope of the present invention.

### Detection of Amplification Products

It should also be understood herein that the scope of the invention is not limited to a specific detection technology. Classically, detection of amplified nucleic acids is performed by standard ethidium bromide-stained agarose gel electrophoresis. Briefly, 10 μL of the amplification mixture are resolved by electrophoresis in a 2% agarose gel containing 0.25 μg/mL of ethidium bromide. The amplicons are then visualized under a UV transilluminator. Amplicon size is estimated by comparison with a molecular weight ladder. It is however clear that other method for the detection of specific amplification products, which may be faster and more practical for routine diagnosis, may be used. Such methods may be based on the detection of fluorescence after or during amplification.

One simple method for monitoring amplified DNA is to measure its rate of formation by measuring the increase in fluorescence of intercalating agents such as ethidium bromide or SYBR® Green I (Molecular Probes). If a more specific detection is required, fluorescence-based technologies can monitor the appearance of a specific product during the nucleic acid amplification reaction. The use of dual-labeled fluorogenic probes such as in the TaqMan™ system (Applied Biosystems) which utilizes the 5′-3′ exonuclease activity of the Taq polymerase is a good example (Livak K. J. et al., 1995, PCR Methods Appl. 4:357-362). TaqMan™ probes are used during amplification and this “real-time” detection is performed in a closed vessel hence eliminating post-PCR sample handling and consequently preventing the risk of amplicon carryover.

Several other fluorescence-based detection methods can be performed in real-time. Examples of such fluorescence-based methods include the use of adjacent hybridization probes (Wittwer, C. T. et al., 1997, BioTechniques 22:130-138), molecular beacon probes (Tyagi S. and Kramer F. R., 1996, Nat. Biotech. 14:303-308) and scorpion probes (Whitcombe, D. et al., 1999, Nat. Biotechnol. 17:804-807). Adjacent hybridization probes are designed to be internal to the amplification primers. The 3′ end of one probe is labelled with a donor fluorophore while the 5′ end of an adjacent probe is labelled with an acceptor fluorophore. When the two probes are specifically hybridized in closed proximity (spaced by 1 to 5 nucleotides) the donor fluorophore which has been excited by an external light source emits light that is absorbed by a second acceptor that emit more fluorescence and yields a fluorescence resonance energy transfer (FRET) signal. Molecular beacon probes possess a stem-and-loop structure where the loop is the probe and at the bottom of the stem a fluorescent moiety is at one end while a quenching moiety is at the other end. The molecular beacons undergo a fluorogenic conformational change when they hybridize to their targets hence separating the fluorophore from its quencher. The FRET principle has been used for real-time detection of PCR amplicons in an air thermal cycler equipped with a built-in fluorometer (Wittwer, C. T. et al., 1997, BioTechniques 22:130-138). Apparatus for real-time detection of PCR amplicons are capable of rapid PCR cycling combined with either fluorescent intercalating agents such as SYBR® Green I or FRET detection. Methods based on the detection of fluorescence are particularly promising for utilization in routine diagnosis as they are very simple, rapid and quantitative.

An exemplary embodiment of amplification conditions is provided in the Example section. However, as used herein the term “amplification condition” refers to temperature and/or incubation time suitable to obtain a detectable amount of the target. Therefore, the term “similar amplification conditions” means that the assay may be performed, if desired, under similar temperature for each target. The term “similar amplification conditions” also means that the assay may be performed, if desired, under similar incubation time for each target. The term “similar amplification conditions” may in some instances also refer to the number of amplification cycles. However, it is well known in the art that number of cycles is not always critical. For example, some samples may be removed before the others or left for additional amplification cycles. In other instances, the term “similar amplification conditions” may also refer to the nature of buffer and amplification reagents used (enzyme, nucleotides, salts, etc.). The term “similar amplification conditions” also means that the conditions (e.g., time, buffer, number of cycles, temperature, or other parameters) may be varied slightly or may be the same.

Exemplary embodiments of detection conditions are provided in the Example section. However, as used herein the term “similar detection condition” refers to temperature and/or incubation time, nature of the signal detected (e.g., fluorescence emission, emission spectra, etc.) or other parameters suitable to obtain a detectable signal. The term “similar detection conditions” also means that the conditions may be varied slightly or may be the same.

Exemplary embodiments of hybridization conditions are provided in the Example section. As used herein the term “similar hybridization conditions” means that the hybridization assay may be performed, if desired, under similar temperature for each target. The term “similar hybridization conditions” also means that the assay may be performed, if desired, under similar incubation time for each target. The term “similar hybridization conditions” may also refer to the nature of the hybridization solution used (salts, stringency, etc.). The term “similar hybridization conditions” also means that the conditions (e.g., time, solution, temperature, or other parameters) may be varied slightly or may be the same.

Amplicon detection may thus be performed by hybridization using species-specific internal DNA probes hybridizing to an amplification product. Such probes may be designed to specifically hybridize to amplicons using the primers described herein. The oligonucleotide probes may be labeled with biotin or with digoxigenin or with any other reporter molecule. In a preferred embodiment, the primers described in the present invention are labeled with Cy3 fluorophores. Hybridization onto a solid support is amenable to miniaturization. However, hybridization in liquid assays or onto solid or semi-solid support, is encompassed herewith.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so.

Detection may also be performed by hybridization technology. For example, detection and identification of pathogens may be performed by sequencing. Simultaneous amplification and detection of nucleic acid material may also be performed using real-time PCR. Detection in liquid assays or solid phase assays (chips, arrays, beads, films, membranes etc.) is also encompassed herewith.

Microarrays of oligonucleotides represent a technology that is highly useful for multiparametric assays. Available low to medium density arrays (Heller, M. J. et al., pp. 221-224. In: Harrison, D. J., and van den Berg, A., 1998, Micro total analysis systems '98, Kluwer Academic Publisher, Dordrecht) could specifically capture fluorescent-labelled amplicons. Detection methods for hybridization are not limited to fluorescence; potentiometry, colorimetry and plasmon resonance are some examples of alternative detection methods. In addition to detection by hybridization, nucleic acid microarrays could be used to perform rapid sequencing by hybridization. Mass spectrometry could also be applicable for rapid identification of the amplicon or even for sequencing of the amplification products (Chiu, N. H. and Cantor, C. R., 1999, Clin. Chem. 45:1578; Berkenkamp, S. et al., 1998, Science 281:260-262).

Probes (i.e., capture probes) targeting internal regions of the PCR amplicons generated using the amplification primer sets described above were therefore designed.

Capture probes can be used either for real-time PCR detection (e.g. TaqMan probes, molecular beacons), for solid support hybridization (e.g. microarray hybridization, magnetic bead-based capture of nucleic acids) or else.

Exemplary embodiments of probes are provided in Table 2. However, a person of skill in the art will understand that other probes may be designed to detect the PCR amplicons generated using the primer pairs of Table 1 although with various efficiency or specificity. As such, the identity of the probe is not limited to the list provided in Table 2 but also extend to any probe which may be capable of specific binding with other regions of the PCR amplicon, including the sense or antisense strand of the PCR amplicon.

For the future of the assay format, integration of steps including sample preparation, genetic amplification, detection, and data analysis into a μTAS are also considered (Anderson, R. C. et al., pp. 11-16. In: Harrison, D. J., and van den Berg, A., 1998, Micro total analysis systems '98, Kluwer Academic Publisher, Dordrecht). In yet another embodiment, the probes described in this invention could be used without the need of prior PCR amplification. Promising ultra-sensitive detection technologies such as the use of polymeric biosensors based on the optical properties of the nucleic acid/polymer complex (Najari, A. et al., 2006, Anal. Chem. 78:7896-7899; Doré, K. et al., 2006, J. Fluoresc. 16:259-265; Ho, H.-A. et al., 2005 J. Am. Chem. Soc. 127:12673-12676; Doré, K. et al., 2004, J. Am. Chem. Soc. 126:4240-4244; Ho, H.-A. et al., 2002, Angew. Chem. Int. Ed. 41:1548-1551) could allow capture and detection of target pathogen species using hybridization probes, without the need for prior PCR amplification.

### Multiplex PCR Amplification

PCR reactions may be performed in mixture containing template genomic DNA preparation obtained for each of the microbial species and diluted at the desired concentration, a buffer suitable for amplification using desired polymerases, primers at a predetermined concentration, dinucleotide triphosphate (dNTPs) mix and DNA polymerase. In order to minimize nucleic acid contamination levels from reagents and solutions, stock solutions may be filtered and solutions may be sterilized and exposed to UV (e.g., using a Spectrolinker™XL-1000 (Spectronics Corp.) between 9999 and 40 000 μJ/cm²). UV exposure may be adjusted as described in patent application WO 03087402A1. An internal control designed to monitor amplification efficiency may be added in the multiplex assay(s). Amplification runs may also include no template (negative) control reactions. Amplification may be performed in any thermal cycler. The amplification conditions typically include a step of denaturation of the nucleic acid where suitable denaturation conditions are used, a step of hybridization (annealing) where suitable hybridization conditions are used, a step of extension where suitable extension conditions by the polymerase are used. The amplicons were typically melted between a range of 60° to 95° C. As known by the person skilled in the art, reaction chemistry and cycling conditions may vary and may be optimized for different PCR reagents combinations and thermocycling devices.

### Microarray Hybridization

Typically, double-stranded amplification products are denatured at 95° C. for 1 to 5 min, and then cooled on ice prior to hybridization. Since double-stranded amplicons tend to reassociate with their complementary strand instead of hybridizing with the probes, an exemplary embodiment of the invention uses single-stranded nucleic acids for hybridization. One such method to produce single-stranded amplicons is to digest one strand with the exonuclease from phage Lambda. Preferential digestion of one strand can be achieved by using a 5′-phosphorylated primer for the complementary strand and a fluorescently-labelled primer for the target strand (Boissinot K. et al., 2007, Clin. Chem. 53:2020-2023). Briefly, amplicons generated with such modified primers were digested by adding 10 units of Lambda exonuclease (New-England Biolabs) directly to PCR reaction products and incubating them at 37° C. for 5 min. Such digested amplification products can be readily used for microarray hybridization without any prior heat treatment.

Microarrays are typically made by pinspotting oligonucleotide probes onto a glass slide surface but the person skilled in the art knows that other surfaces and other methods to attach probes onto surfaces exist and are also covered by the present invention. Lateral flow microarrays represent an example of recent rapid solid support hybridization technology (Carter, D. J. and Cary, R. B., 2007, Nucleic Acids Res. 35:e74). For the illustrative example described below, oligonucleotide probes modified with a 5′ amino-linker were suspended in Microspotting solution plus (TeleChem International) and spotted at 30 μM on Super Aldehyde slides (Genetix) using a VIRTEK SDDC-2 Arrayer (Bio-Rad Laboratories). In addition to DNA or RNA oligonucleotides, nucleotide analogs such as peptide nucleic acids (PNA), locked nucleic acids (LNA) and phosphorothioates can be used as probes and are also the object of this invention.

Typically hybridization of the target nucleic acid is performed under moderate to high stringency conditions. Such high stringency conditions allow a higher specificity of the interaction between the probe and target. Hybridization may be performed at room temperature (19-25° C.) using probes attached to a solid support and hybridization solution containing amplicons. Active hybridization may be achieved using a microfluidic device, where the hybridization solution containing the amplicon are flowed above the microarray. Washing step may be performed with solutions allowing hybridization at varying stringencies. The microfluidic version of the procedure is typically performed within 15 min including the washing and rinsing steps. A person of skill in the art is well aware that nucleic acid hybridization and washing conditions can be modified and still achieve comparable levels of sensitivity and specificity as long as the overall process results in comparable stringency for nucleic acid recognition.

An advantage of the present invention is that all microarray hybridizations and washing procedures may be performed under uniform conditions for all probes using the four multiplex amplification combinations.

Slides may be scanned and the hybridization signals may be quantified using suitable apparatus such as a ScanArray 4000XL (PerkinElmer) or a G2505B Microarray Scanner (Agilent) and Genepix 6 (MDS Analytical Technologies). All hybridization signals may be corrected for background signal and expressed as a percentage of a control oligonucleotide signal.

Identification of hybridized species may be performed using previously obtained reference hybridization data, from which are determined specific probe patterns and hybridization statistics. Probe patterns may readily identify hybridized species since a specific probe pattern is a set of one or more probes that will all generate a unique hybridization signal together for a given species. By contrast, hybridization statistics allow for probabilistic inference (either Bayesian or other inference methods) of what species are more likely to have hybridized. Positive hybridization signals as well as negative hybridization signals can be taken into account for microarray data analysis. Further analytical refinements such as machine learning methods could also be used for interpreting hybridization data.

Other aspects of the invention relate to kits which may comprise an oligonucleotide described herein.

In an exemplary embodiment, the kit may comprise a plurality of oligonucleotides for the specific amplification of a genetic material from a pathogen selected from the group consisting of Acinetobacter baumannii, Acinetobacter Iwoffii, Aeromonas caviae, Aeromonas hydrophila, Bacillus cereus, Bacillus subtilis, Citrobacter braakii, Citrobacter freundii, Citrobacter koseri, Enterobacter aerogenes, Enterobacter cloacae, Enterobacter sakazakii, Enterococcus faecium, Gemella haemolysans, Gemella morbillorum, Haemophilus influenzae, Kingella kingae, Klebsiella oxytoca, Klebsiella pneumoniae, Morganella morganii, Neisseria gonorrhoeae, Neisseria meningitidis, Pasteurella multocida, Propionibacterium acnes, Proteus mirabilis, Providencia rettgeri, Pseudomonas aeruginosa, Salmonella choleraesuis, Serratia liquefaciens, Serratia marcescens, Streptococcus agalactiae, Streptococcus anginosus, Streptococcus bovis, Streptococcus mutans, Streptococcus salivarius, Streptococcus sanguinis, Streptococcus suis, Vibrio vulnificus, Yersinia enterocolitica, Yersinia pestis/Yersinia pseudotuberculosis, Enterococcus faecalis, Clostridium perfringens, Corynebacterium jeikeium, and Capnocytophaga canimorsus.

In another exemplary embodiment, the kit may comprise a plurality of oligonucleotides for the specific amplification of a genetic material from a pathogen selected from the group consisting of Citrobacter freundii, Citrobacter koseri, Enterobacter aerogenes, Enterobacter cloacae, Enterobacter sakazakii, Klebsiella oxytoca, Klebsiella pneumoniae, Salmonella choleraesuis, Listeria monocytogenes, Pasteurella pneumotropica, Staphylococcus aureus, Staphylococcus epidermidis, Staphylococcus haemolyticus, Staphylococcus hominis, Staphylococcus saccharolyticus, Staphylococccus saprophyticus, Staphylococcus warneri, Streptococcus dysgalactiae, Streptococcus pneumoniae, and Streptococcus pyogenes.

In a further exemplary embodiment, the kit may comprise a plurality of oligonucleotides for the specific amplification of a genetic material from a pathogen selected from the group consisting of Candida albicans, Candida glabrata, Candida parapsilosis, Candida tropicalis, Candida krusei, Aspergillus fumigatus, Aspergillus niger, Aspergillus nidulans, Aspergillus flavus, and Aspergillus terreus.

In yet another exemplary embodiment, the kit may comprise a plurality of oligonucleotides for the specific amplification of a genetic material from a pathogen selected from the group consisting of Bacteroides fragilis, Brucella melitensis, Burkholderia cepacia, Stenotrophomonas maltophilia, Escherichia coli and Shigella sp.

In accordance with the present invention, the kit may comprise oligonucleotides for the amplification of each of the pathogen species or one of the four group listed above.

Also in accordance with the present invention, the kit may further comprise in a separate container or attached to a solid support, an oligonucleotide for the detection of each of the pathogen species.

In accordance with the present invention, the oligonucleotides may be provided in separate containers where each may comprise individual oligonucleotides. The container may also comprise a specific primer pair. The oligonucleotides may be provided in a single container comprising a mixture of oligonucleotides for amplification of each desired genetic material.

In another aspect, the present invention relates to a kit comprising probes for the detection of the pathogen species listed in Table 4. In accordance with an embodiment of the invention, the kit may comprise probes for the detection of each of the pathogen species listed in Table 4. In accordance with another embodiment of the invention, the kit may comprise probes which are particularly useful for detection/identification purposes.

The present invention relates in a further aspect to an array which may comprise a solid substrate (support) and a plurality of positionally distinguishable probes attached to the solid substrate (support). Each probe comprises a different nucleic acid sequence and may be capable of specific binding to a pathogen selected from the group consisting of those listed in Table 4.

In accordance with the present invention, each probe may independently comprise from 10 to 50 nucleotides.

More particular aspects of the invention relate to an array which may comprise:
a) at least one member selected from the group consisting of an oligonucleotide comprising from 0 to 5 nucleotide addition and/or deletion to SEQ ID NO: 27 to SEQ ID NO: 125, SEQ ID NO: 131 to SEQ ID NO. 202 or SEQ ID NO: 203 or to a complement thereof and wherein the addition and/or deletion is located at a 5′ end and/or 3′ end of the nucleic acid sequence;
b) at least one member selected from the group consisting of an oligonucleotide comprising from 0 to 5 nucleotide addition and/or deletion to SEQ ID NO: 204 to SEQ ID NO: 237, SEQ ID NO: 241 to SEQ ID NO. 293 or SEQ ID NO: 364 or to a complement thereof and wherein the addition and/or deletion is located at a 5′ end and/or 3′ end of the nucleic acid sequence;
c) at least one member selected from the group consisting of an oligonucleotide comprising from 0 to 5 nucleotide addition and/or deletion to SEQ ID NO: 294 to SEQ ID NO: 332 or SEQ ID NO: 333 or to a complement thereof and wherein the addition and/or deletion is located at a 5′ end and/or 3′ end of the nucleic acid sequence;
d) at least one member selected from the group consisting of an oligonucleotide comprising from 0 to 5 nucleotide addition and/or deletion to SEQ ID NO: 339 to SEQ ID NO: 352, SEQ ID NO: 356, SEQ ID NO: 357, SEQ ID NO: 366 to SEQ ID NO: 373 or SEQ ID NO: 374 or to a complement thereof and wherein the addition and/or deletion is located at a 5′ end and/or 3′ end of the nucleic acid sequence;
wherein each oligonucleotide is attached to a solid support and wherein each oligonucleotide is located at an addressable position.

It has been found that subgroups of probes are suitable to carry the detection. For example, in a specific embodiment the oligonucleotide may be selected from the group consisting of:
a) an oligonucleotide having or consisting of the sequence selected from the group consisting of SEQ ID NO: 27 to SEQ ID NO: 44, SEQ ID NO: 46 to SEQ ID NO: 63, SEQ ID NO: 65 to SEQ ID NO: 71, SEQ ID NO: 73 to SEQ ID NO: 77, SEQ ID NO: 79 to SEQ ID NO: 97, SEQ ID NO: 99 to SEQ ID NO: 125, SEQ ID NO: 131 to SEQ ID NO. 202 and SEQ ID NO: 203;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

In another particular embodiment, the oligonucleotide may be selected from the group consisting of:
a) an oligonucleotide having or consisting of the sequence selected from the group consisting of SEQ ID NO: 204, SEQ ID NO: 208, SEQ ID NO: 211, SEQ ID NO: 212, SEQ ID NO: 214, SEQ ID NO: 215, SEQ ID NO: 219, SEQ ID NO: 223, SEQ ID NO: 226, SEQ ID NO: 227, SEQ ID NO: 229, SEQ ID NO: 231, SEQ ID NO: 233, SEQ ID NO: 236, SEQ ID NO: 241, SEQ ID NO: 242, SEQ ID NO: 244, SEQ ID NO: 246, SEQ ID NO: 248, SEQ ID NO: 249, SEQ ID NO: 253 to SEQ ID NO: 256, SEQ ID NO: 261, SEQ ID NO: 264 to SEQ ID NO: 267, SEQ ID NO: 270, SEQ ID NO: 272, SEQ ID NO: 279 to SEQ ID NO: 281, SEQ ID NO: 284 to SEQ ID NO: 288, SEQ ID NO: 291, SEQ ID NO: 292 and SEQ ID NO: 364;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

In yet another particular embodiment, the oligonucleotide may be selected from the group consisting of:
a) an oligonucleotide having or consisting of the sequence selected from the group consisting of SEQ ID NO: 294, SEQ ID NO: 296 to SEQ ID NO: 309, SEQ ID NO: 312, SEQ ID NO: 314, SEQ ID NO: 316, SEQ ID NO: 317, SEQ ID NO: 318, SEQ ID NO: 320 to SEQ ID NO: 323, SEQ ID NO: 326 to SEQ ID NO: 330 and SEQ ID NO: 332;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

In another particular embodiment, the oligonucleotide may be selected from the group consisting of:
a) an oligonucleotide having or consisting of the sequence selected from the group consisting of SEQ ID NO: 339 to SEQ ID NO: 344, SEQ ID NO: 348, SEQ ID NO: 366 to SEQ ID NO: 373 and SEQ ID NO: 374;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

The present invention method for the diagnosis of a bloodstream infection in an individual in need, the method comprising detecting the presence or absence of a pathogen from a sample obtained from the individual with oligonucleotides capable of specific binding with genetic material of a pathogen selected from the group consisting of those listed in Table 4, wherein the genetic material is detected with any one or all of SEQ ID NO: 375, SEQ ID NO: 376, SEQ ID NO: 377 or SEQ ID NO: 378 and with an oligonucleotide selected from the group consisting of any one of SEQ ID NO: 1 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373 and SEQ ID NO: 374. The presence of the pathogen in the test sample (presence of the genetic material of the pathogen) may thus be indicative of a bloodstream infection associated with the pathogen detected. By carrying out the method of the present invention, the pathogen(s) present in a test sample, may thus be suitably identified. As such, appropriate treatment of the patient may be initiated.

In accordance with the present invention, the genetic material may be detected with an oligonucleotide selected from the group consisting of any one of SEQ ID NO: 1 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373 and SEQ ID NO: 374.

The present invention also relates in an additional aspect to a library of oligonucleotides comprising at least two oligonucleotides described herein.

In accordance with the present invention, each oligonucleotide may be provided in a separate container or may be attached to a solid support.

In an exemplary embodiment of the invention, the library may comprise,
a) an oligonucleotide having or consisting of the sequence selected from the group consisting of SEQ ID NO: 27 to SEQ ID NO: 125, SEQ ID NO: 131 to SEQ ID NO: 202 or SEQ ID NO: 203;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and/or a deletion of from 0 to 5 nucleotides at the other of a 5′ end or 3′ end thereof, and;
e) a complement of any one of the above.

In another exemplary embodiment of the invention, the library may comprise,
a) an oligonucleotide having or consisting of the sequence selected from the group consisting of SEQ ID NO: 204 to SEQ ID NO: 237, SEQ ID NO: 241 to SEQ ID NO: 293 or SEQ ID NO: 364;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and/or a deletion of from 0 to 5 nucleotides at the other of a 5′ end or 3′ end thereof, and;
e) a complement of any one of the above.

In a further exemplary embodiment of the invention, the library may comprise,
a) an oligonucleotide having or consisting of the sequence selected from the group consisting of SEQ ID NO: 294 to SEQ ID NO: 332 or SEQ ID NO: 333;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and/or a deletion of from 0 to 5 nucleotides at the other of a 5′ end or 3′ end thereof, and;
e) a complement of any one of the above.

In an additional exemplary embodiment of the invention, the library may comprise:
a) an oligonucleotide having or consisting of the sequence selected from the group consisting of SEQ ID NO: 339 to SEQ ID NO: 352, SEQ ID NO: 356, SEQ ID NO: 357, SEQ ID NO: 366 to SEQ ID NO: 373 or SEQ ID NO: 374;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and/or a deletion of from 0 to 5 nucleotides at the other of a 5′ end or 3′ end thereof, and;
e) a complement of any one of the above.

In accordance with the present invention, the oligonucleotide of the library may comprise a label.

In accordance with the present invention, the oligonucleotide of the library may be attached to a solid support.

The present invention is illustrated in further details by the following non-limiting examples.

### EXAMPLES

### Example 1

### Amplification and detection of 73 sepsis-associated Bacterial and Fungal Species

The four multiplex PCR assays were tested using the DNA amplification apparatus Rotor-Gene™ (Corbett Life Science). These multiplex PCR tests incorporate primers specific to tuf, recA, and/or tef1 gene sequences. All PCR reactions were performed in a 25 μL mixture containing 1 μL of purified template genomic DNA preparation previously obtained for each of the 73 species (Table 4) tested and diluted at the desired concentrations, 1×PC2 buffer (Ab Peptides, inc.), (1×PC2 is 50 mM Tris-HCl at pH 9.1, 16 mM (NH₄)₂SO₄, 3.5 mM MgCl₂, 0.150 mg/mL Bovine serum albumin), supplemented with MgCl₂ (Promega) so the final magnesium chloride concentration is 4.5 mM, supplemented with bovine serum albumin fraction V (Sigma) so the final BSA concentration is 2.15 mg/mL, 0.4 to 1.2 μM of each HPLC-purified primers (optimal concentration for each primer was adjusted to ensure maximum amplification yield), 0.2 mM of the four dinucleotide triphosphate (dNTPs) mix (GE Healthcare) and 0.05 U/μL of Klentaq® DNA polymerase (Ab Peptides, inc), coupled with TaqStart® antibody for the Hot Start procedure (Clontech). Whenever possible, to minimize nucleic acid contamination levels from reagents and solutions, stock solutions were filtered on 0.1 μm polyethersulfone membranes (Pall). In addition to 0.1 μm filtration, water and TE were also autoclaved. 8-methoxypsoralen (8-Mop) (Sigma) was added to the reaction master mix at 0.13 μg/μL and exposed to UV illumination in a Spectrolinker™XL-1000 (Spectronics Corp.) at 30 000 μJ/cm2 in order to control DNA contamination. For each of the four multiplex combinations, 10 to 25 copies of an internal control designed to monitor amplification efficiency was added following the UV treatment. These controls are built using a tag sequence not related to the targeted genes flanked by sequences complementary to two of the primer sequences present in the multiplex mixture. Design and use of such amplification internal controls have been previously described (Ke, D. et al., 2000, Clin. Chem. 46:324-331; Hoorfar, J. et al., 2004, APMIS 112:808-814; Hoorfar J. et al., 2004, J. Clin. Microbiol. 42:1863-1868). All amplification runs also included no template (negative) control reactions in which DNA-free water or TE 1× were used as template. For post-PCR detection of amplicons directly in the thermocycler apparatus, the PCR mixture described above was supplement with 1× SYBR® Green (Molecular Probes), and the different amplicons were distinguished by melting curves analysis. Uniform cycling conditions for the Rotor-Gene™ apparatus were: 1 min at 95° C., followed by 40 cycles of 1 sec at 95° C., 10 sec at 60° C., and 20 sec at 72° C. The amplicons were melted between a range of 60° to 95° C. The analytical sensitivity of the multiplex PCR assays was determined by testing a range between 10 000 and 3 genome copies equivalent for the 73 species (Table 4).

Multiplex number one comprised primers SEQ ID NOs: 375 and 376 (corresponding to SEQ ID NOs: 636 and 637 of international patent application NO. PCT/CA00/01150) and SEQ ID NOs: 1 to 8. All primers were used at 1 μM except for SEQ ID NOs: 3 and 4 which were at 0.4 μM.

Multiplex number two comprised primers SEQ ID NOs: 9 to 14. All primers were used at 1.2 μM except for SEQ ID NOs: 9 and 10 which were at 1 μM.

Multiplex number three comprised primers SEQ ID NOs: 15 to 21. Primers SEQ ID NOs: 15 to 17 were used at 1 μM and SEQ ID NOs: 18 to 21 were used at 0.8 μM.

Multiplex number four (version 1) comprised primers SEQ ID NOs: 22 to 25 and primers SEQ ID NOs: 377 and 378 (corresponding to SEQ ID NOs: 1661 and 1665 of international patent application NO. PCT/CA00/01150). All primers were used at 0.6 μM except for SEQ ID NOs: 22 and 23 which were at 1.0 μM.

Results of these experiments indicate that the detection limit for the 73 bacterial and fungal species tested (Table 4) ranged from 3 to 50 copies of microbial genome per PCR reaction. Furthermore, for each multiplex PCR combinations, the specificity of the PCR assays was verified using 10 000 copies of concentrated human genomic DNA. No amplification product could be detected.

The above conditions thus allowed the amplification and detection of 73 sepsis-associated bacterial and fungal species with combinations of PCR primers in four multiplex formats using uniform amplification conditions coupled with post-PCR SYBR Green I melting curve analysis for amplicon detection.

### Example 2

### Detection and Identification of 73 Bacterial and Fungal Species Using Microarrays

PCR were carried out as in Example 1, except that for each primer pair, one primer was phosphorylated at its 5′ end while the other member of the pair was labelled with Cy-3 at its 5′ end. Amplicons generated with such modified primers were digested by adding 10 units of Lambda exonuclease (New-England Biolabs) directly to PCR reaction products and incubating them at 37° C. for 5 min (Boissinot K. et al., 2007, Clin. Chem. 53:2020-2023). Such digested amplification products were readily used for microarray hybridization without any prior heat treatment. 4.8 μL of digested amplicons were diluted in hybridization solution so that the resulting solution is 6×SSPE (OmniPur; EM Sciences), 0.03% polyvinylpyrrolidone, 30% formamide, 5 nM hybridization control Cy3-labelled oligonucleotide bbc1 (GAGTATGGTCTGCCTATCCT), 0.5 μM hybridization control Cy5-labelled oligonucleotide bbc2 (ACACTGCGATGCGTGATGTA) in a total volume of 20 μL. The whole 20 μL volume was subjected to passive hybridization. Passive hybridization (1 h) was performed at room temperature (19-25° C.) using a glass lifterslip (Erie Scientific) apposed to the microarray slide with 20 μL of hybridization solution containing amplicons. Each probe was thus spotted to a specific and identifiable location. Washing step was performed in 0.2× SSPE containing 0.1% Sodium dodecyl-sulfate, followed by rinsing in 0.2× SSPE. Slides were scanned using a ScanArray 4000XL (PerkinElmer) or a G2505B Microarray Scanner (Agilent) and the hybridization signals were quantified using Genepix 6 (MDS Analytical Technologies). All hybridization signals were corrected for background signal and were then expressed as a percentage of a control oligonucleotide signal.

Amplicons produced by multiplex PCR number one were hybridized on microarray using probe combinations SEQ ID NOs: 27 to 203.

Amplicons produced by multiplex PCR number two were hybridized on microarray using probe combinations SEQ ID NOs: 204 to 293.

Amplicons produced by multiplex PCR number three were hybridized on microarray using probe combinations SEQ ID NOs: 294 to 338.

Amplicons produced by multiplex PCR number four (version 1) were hybridized on microarray using probe combinations SEQ ID NOs: 339 to 363.

Results of these experiments indicate that the analytical sensitivity with the microarray detection ranged from 10 to 50 copies of microbial genome per PCR reaction for each of the 73 bacterial and fungal species tested with the four multiplex PCR combinations either by using hybridization pattern analysis and/or statistical inference analysis of hybridization signals.

Specificity with the microarray detection was verified by the amplification of each of the 73 bacterial and fungal species with the four multiplex PCR combinations using concentrated (1 to 5 ng) genomic DNA. Identification of the template DNA is realized either by using hybridization pattern analysis and/or statistical inference analysis of hybridization signals. At some high concentration of target nucleic acids, it was sometimes not always easy to distinguish between closely related Enterobacteriaceae species. Therefore, robustness of identification might be improved by selecting more discriminant (see Examples 3-5) sequences regions to distinguish between Escherichia coli, Citrobacter freundii and Salmonella choleraesuis.

The specificity of the assay was verified with 10 000 copies of concentrated human genomic DNA as described in Example 1 and no hydridization signal could be detected with the human templates.

Therefore, the capture probes used for microarray hybridization allowed specific, sensitive, and ubiquitous detection as well as identification of amplicons generated by PCR from the 73 bacterial and fungal species tested, under the above experimental conditions.

### Example 3

### Assay Improvement—Amplification of Pathogens' Nucleic Acids

The four multiplex PCR assays were carried out as described in Example 1 except that primers combination in multiplex four (version 1) was modified to improve specific detection of Escherichia coli using probe combinations on microarray (see Example 4). PCR were also carried out with a higher internal control copy number (25 to 40 copies) to increase the hybridization signal on microarrays (see example 4). The analytical sensitivity of the multiplex PCR assays was determined by testing a range between 10 000 and 10 genome copies equivalent for each species.

All multiplex PCR comprised the same primer combinations described in Example 1 except for multiplex number four where primers SEQ ID NOs: 24 and 25 were omitted in the primer combination and primers SEQ ID NOs: 377 and 378 were replaced by primer SEQ ID NO: 26. All primers were used at 1 μM. Detection was performed as described in Example 1.

Results of these experiments indicate that the detection limit for the 73 bacterial and fungal species tested ranged from 10 to 50 copies of microbial genome per PCR reaction. For each multiplex PCR combination, the specificity of the PCR assay was verified using 10 000 copies of concentrated human genomic DNA. No amplification product could be detected.

The four multiplex PCR assays allowed the sensitive and ubiquitous amplification of 73 bacterial and fungal species when coupled with post-PCR SYBR Green I melting curve analysis for amplicon detection.

### Example 4

### Assay Improvement—Detection of Pathogens' Nucleic Acids Using Microarrays

PCR were carried out as described in Example 3, except that for each primer pair, one primer was phosphorylated at its 5′ end while the other member of the pair was labelled with Cy-3 at its 5′ end. Digestion of the amplicons by Lambda exonuclease, passive hybridization on microarray and signal acquisition were carried out as in Example 2.

Amplicons produced by multiplex PCR number one were hybridized on microarray using probe combinations SEQ ID NOs: 27 to 203.

Amplicons produced by multiplex PCR number two were hybridized on microarray using probe combinations SEQ ID NOs: 204 to 293, 364 and 365.

Amplicons produced by multiplex PCR number three were hybridized on microarray using probe combinations SEQ ID NOs: 294 to 338.

Amplicons produced by multiplex PCR number four (version 2) were hybridized on microarray using probe combinations SEQ ID NOs: 339 to 363 and 366 to 374.

Results of these experiments indicate that the analytical sensitivity with the microarray detection was 10 to 100 copies of microbial genome for each of the 73 bacterial and fungal species tested with the four multiplex PCR combinations either by using hybridization pattern analysis and/or statistical inference analysis of hybridization signals.

The specificity with the microarray detection was verified by the amplification of each of the 73 bacterial and fungal species with the four multiplex PCR combinations using concentrated (1 to 5 ng) genomic DNA. Identification of the template DNA is realized either by using hybridization pattern analysis and/or statistical inference analysis of hybridization signals.

The specificity of the assay was verified with 10 000 copies of concentrated human genomic DNA as described in Example 1 and no hydridization signal could be detected with the human templates.

The specificity of the assay was also verified with 130 other closely related pathogenic species. 1 ng of genomic DNA was added to multiplexes PCR reaction and hybridized on their specific microarray when an amplicon was detected by post-PCR SYBR Green I melting curve analysis. Cross-hybridization signals have been included in the hybridization pattern analysis and/or statistical inference analysis of hybridization signals to improved identification of bacterial and fungal species targeted by the assay.

The capture probes used in microarray hybridization allowed specific, sensitive, and ubiquitous detection as well as identification of amplicons generated by PCR from the 73 bacterial and fungal species tested.

### Example 5

### Detection and Identification of Pathogens Using a Microfluidic Hybridization Automated System and Microarrays

In an exemplary embodiment, active hydridization with only multiplex 3 and multiplex 4 (version 2) was performed.

PCR were carried out as described in Example 3, except that for each primer pair, one primer was phosphorylated at its 5′ end while the other member of the pair was labelled with Cy-3 at its 5′ end. Digestion of amplicon by Lambda exonuclease was carried out as in Example 2. Such digested amplification products were readily used for microarray hybridization without any prior heat treatment. 4.8 μL of digested amplicons were diluted in hybridization solution so that the resulting solution is 6×SSPE (OmniPur; EM Sciences), 0.03% polyvinylpyrrolidone, 30% formamide, 5 nM hybridization control Cy3-labelled oligonucleotide bbc1 (GAGTATGGTCTGCCTATCCT), 0.5 μM hybridization control Cy5-labelled oligonucleotide bbc2 (ACACTGCGATGCGTGATGTA) in a total volume of 20 μL. 2 μL was subjected to active hybridization. Active hybridization (5 min) was achieved using a CD-based poly-dimethylsiloxane microfluidic device, flowing the solution above the microarray at room temperature (19-25° C.) as previously described (Peytavi, R. et al., 2005, Clin. Chem. 51:1836-1844). Washing step was performed in 0.2× SSPE containing 0.1% Sodium dodecyl-sulfate, followed by rinsing in 0.2× SSPE. The microfluidic version of the procedure can be performed within 15 min including the wash and rinse steps. Slides were scanned using a ScanArray 4000XL (PerkinElmer) or a G2505B Microarray Scanner (Agilent) and the hybridization signals were quantified using Genepix 6 (MDS Analytical Technologies). All hybridization signals were corrected for background signal and were then expressed as a percentage of a control oligonucleotide signal.

Amplicons produced by multiplex PCR number three were hybridized on microarray using probe combinations SEQ ID NOs: 294, 296 to 309, 312, 314, 316, 317, 318, 320 to 323, 326 to 330, 332 and 335.

Amplicons produced by multiplex PCR number four (version 2) were hybridized on microarray using probe combinations SEQ ID NOs: 339 to 344, 348, 353, and 366 to 374.

Analytical sensitivity with the microarray detection was 10 copies of microbial genome for each of the 10 fungal species amplified by multiplex PCR three and 10 to 25 copies of microbial genome for each of the 5 bacterial species amplified by multiplex PCR four (version 2).

Specificity with the microarray detection was verified by amplification of 5 bacterial and 10 fungal species with the multiplex PCR number three and four (version 2) using concentrated (1 to 5 ng) genomic DNA. Identification of the template DNA was realized either by using hybridization pattern analysis and/or statistical inference analysis of hybridization signals.

The specificity of the assay was verified with 40 other closely related pathogenic species. 1 ng of genomic DNA was added to the PCR reactions and hybridized on their respective microarray when an amplicon was detected by post-PCR SYBR Green I melting curve analysis. Cross-hybridization signals have been included in the hybridization pattern analysis and/or statistical inference analysis of hybridization signals to improved identification of bacterial and fungal species targeted by the assay.

The capture probes used in microarray hybridization allowed specific, sensitive, and ubiquitous detection as well as identification of amplicons generated by PCR from the 5 bacterial and 10 fungal species tested using the automated CD-based microfluidic hybridization system.

### Example 6

### Identification of Pathogens from Spiked Blood

Specific identification of the most important bloodstream infection pathogens from spiked blood was carried out by multiplex PCR. These pathogens were detected with microfluidic hybridization automated system using microarray and a limited set of probe sequence combinations described below.

Blood samples were spiked with various amounts of culture cells from selected bacterial and fungal pathogens causing bloodstream infection, i.e., Acinetobacter baumannii, Bacteroides fragilis, Citrobacter freundii, Citrobacter koseri, Enterobacter aerogenes, Enterobacter cloacae, Enterococcus faecalis, Enterococcus faecium, Escherichia coli, Haemophilus influenzae, Klebsiella oxytoca, Klebsiella pneumoniae, Proteus mirabilis, Pseudomonas aeruginosa, Serratia marcescens, Staphylococcus aureus, Staphylococcus epidermidis, Staphylococcus haemolyticus, Staphylococcus hominis, Staphylococcus wameri, Stenotrophomonas maltophilia, Streptococcus agalactiae, Streptococcus anginosus, Streptococcus dysgalactiae, Streptococcus mutans, Streptococcus pneumoniae, Streptococcus pyogenes, Streptococcus sanguinis, Aspergillus fumigatus, Candida albicans, Candida glabrata, Candida krusei, Candida parapsilosis, Candida tropicalis.

DNA was extracted by adding 15 mL of lysis solution containing 100 mg/mL of Saponin from Quillaja bark in TE1× to 5 mL of spiked blood sample and mixed for 10 seconds using a vortex set at maximum speed. Subsequently, the solution was centrifuged at 10 000 g for 5 minutes, and the supernatant was discarded. Then, 10 mL of lysis solution was added to the pellet and mixed for 10 seconds using a vortex set at maximal speed. The suspension was then centrifuged at 10 000 g for 5 minutes and the supernatant was discarded. The pellet was washed twice with TE 1× for samples containing bacteria or PBS 1× for samples containing yeast cells. 50 μL of TE 1× (rinsing/harvesting solution) was added to the washed pellet. The washed pellet and TE1× were mixed for 15 seconds using a vortex set at maximum speed. The pellet was removed by using a micropipette tip. The remaining suspension containing the microbial cells was mechanically lysed with glass beads to extract microbial nucleic acids by using the BD GeneOhm™ Lysis Kit (BD Diagnostics-GeneOhm).

PCR were carried out as described in Example 3, except that for each primer pair, one primer was phosphorylated at its 5′ end while the other member of the pair was labelled with Cy-3 at its 5′ end. Digestion of the amplicon by Lambda exonuclease, active hybridization on microarray and signal acquisition were carried out as in Example 2.

Amplicons produced by multiplex PCR number one were hybridized on microarray using probe combinations SEQ ID NOs: 27 to 44, 46 to 63, 65 to 71, 73 to 77, 79 to 97, 99 to 125, 127, 129, and 131 to 203.

Amplicons produced by multiplex PCR number two were hybridized on microarray using probe combinations SEQ ID NOs: 204, 208, 211, 212, 214, 215, 219, 223, 226, 227, 229, 231, 233, 236, 241, 242, 244, 246, 248, 249, 253 to 256, 261, 264 to 267, 270, 272, 279 to 281, 284 to 288, 291, 292, 364, and 365.

Amplicons produced by multiplex PCR number three were hybridized on microarray using probe combinations SEQ ID NOs: 294, 296 to 309, 312, 314, 316, 317, 318, 320 to 323, 326 to 330, 332, and 335.

Amplicons produced by multiplex PCR number four (version 2) were hybridized on microarray using probe combinations SEQ ID NOs: 339 to 344, 348, 353, and 366 to 374.

For 25/28 bacterial species and 4/6 fungal species tested by active microarray hybridization, it was possible to identify the source of the template DNA with a sensitivity of ≦30 CFU/mL of blood while for 3/28 bacterial species and 2/6 fungal species the sensitivity level was ≧31 CFU/mL of blood. Hybridization pattern analysis and/or statistical inference analysis of hybridization signals was performed as described in Example 5.

For each multiplex PCR combination, specificity of the assay was verified using blood samples without spiked microbial cells as described above. No hybridization signal could be detected from these samples.

The capture probes used in this microarray hybridization allowed specific, sensitive, and ubiquitous detection as well as identification of amplicons generated by PCR from various amounts of culture cells spiked in blood samples using the automated CD-based microfluidic hybridization system.

Although the present invention has been described herein by way of exemplary embodiments, it can be modified without departing from the scope and the nature of the invention.

The present description refers to a number of documents, the content of which is herein incorporated by reference in their entirety.

TABLE 1
List of selected amplification primers for the four
multiplex combinations

| | | Ref. No. in | | |
|---|---|---|---|---|
| Multiplex | SEQ ID | WO 2001/ | | Target or source |
| combination | NO. | 023604A2 | Sequence | species |
| Multiplex #1 | 375 | 636 | ACTGGYGTTGAIATGTTCCGYAA | Broad-spectrum * |
| | 376 | 637 | ACGTCAGTIGTACGGAARTAGAA | Broad-spectrum * |
| | 1 | | ACAGGTGTTGAAATGTTCCGTAA | Enterococcus |
| | | | | faecalis |
| | 2 | | ACGTCTGTTGTACGGAAGTAGAA | Enterococcus |
| | | | | faecalis |
| | 3 | | CAGGAATCGAAATGTTCAGAAAG | Clostridium |
| | | | | perfringens |
| | 4 | | ACGTCTGTTGTTCTGAAGTAGAA | Clostridium |
| | | | | perfringens |
| | 5 | | ACCTCCATCGAGATGTTCAACAA | Corynebacterium |
| | | | | jeikeium |
| | 6 | | GGTGGTGCGGAAGTAGAA | Corynebacterium |
| | | | | jeikeium |
| | 7 | | ACAGGAGTTGAGATGTTCCGTAA | Capnocytophaga |
| | | | | canimorsus |
| | 8 | | ACGTCAGTTGTACGAACATAGAA | Capnocytophaga |
| | | | | canimorsus |
| Multiplex #2 | 9 | | GGTWGTIGCTGCGACTGACGG | Broad-spectrum * |
| | 10 | | TCAATCGCACGCTCTGGTTC | Broad-spectrum * |
| | 11 | | AACGTGGTCAAGTWTTAGC | Staphylococcus sp. |
| | 12 | | GTACGGAARTAGAATTGWGG | Staphylococcus sp. |
| | 13 | | GTGGRATIGCIGCCTTTATCG | Streptococcus sp. |
| | 14 | | ATIGCCTGRCTCATCATACG | Streptococcus sp. |
| Multiplex #3 | 15 | | CAAGATGGAYTCYGTYAAITGGGA | Candida sp. |
| | 16 | | CATCTTGCAATGGCAATCTCAATG | Candida sp. |
| | 17 | | CATCTTGTAATGGTAATCTTAATG | Candida krusei |
| | 18 | | GTTCCAGACYICCAAGTATGAG | Aspergillus sp. |
| | 19 | | ATTTCGTTGTAACGATCCTCGGA | Aspergillus sp. |
| | 20 | | GATTTCGTTGTAACGATCCTGAGA | Aspergillus flavus |
| | 21 | | ATTTCGTTGTAACGGTCCTCAGA | Aspergillus terreus |
| Multiplex #4 | 22 | | TGATGCCGRTIGAAGACGTG | Broad-spectrum * |
| | 23 | | AGYTTGCGGAACATTTCAAC | Broad-spectrum * |
| | 24 | | GGCCAGTCCGTCCTCG | Streptomyces |
| | | | | avermitilis |
| | 25 | | GATGCCGGTGACCGTGGT | Streptomyces |
| | | | | avermitilis |
| | 377 | 1661 | TGGGAAGCGAAAATCCTG | Escherichia coli + |
| | | | | Shigella sp. |
| | 378 | 1665 | CAGTACAGGTAGACTTCTG | Escherichia coli + |
| | | | | Shigella sp. |
| | 26 | | GTGGGAAGCGAAAATCCTG | Escherichia coli + |
| | | | | Shigella sp. |

* Broad-spectrum primers where chosen for their capacity to amplify many bacterial species.

TABLE 2
List of selected hybridization probes

| SED ID | | Target species | Preferred |
|---|---|---|---|
| NO. | Sequence | (designed for) | Multiplex |
| 27 | TACTTCTGCGTCGAATTTAG | Acinetobacter baumannii | Multiplex #1 |
| 28 | ACTTCTGCGTCGAATTTA | Acinetobacter baumannii | Multiplex #1 |
| 29 | CTTCTGCGTCGAATTTA | Acinetobacter baumannii | Multiplex #1 |
| 30 | GTAACCATTTAAGAATGGAG | Acinetobacter baumannii | Multiplex #1 |
| 31 | AACCATTTAAGAATGGAG | Acinetobacter baumannii | Multiplex #1 |
| 32 | CACGAAGAAGAACACCACAG | Acinetobacter lwoffii | Multiplex #1 |
| 33 | GAAGAAGAACACCACAG | Acinetobacter lwoffii | Multiplex #1 |
| 34 | TTCACGCTTCACGCCACGCA | Aeromonas caviae | Multiplex #1 |
| 35 | TCACGCTTCACGCCACGC | Aeromonas caviae | Multiplex #1 |
| 36 | CGGTAGCCCTTGAAGAAC | Aeromonas caviae | Multiplex #1 |
| 37 | GGTAGCCCTTGAAGAAC | Aeromonas caviae | Multiplex #1 |
| 38 | CAGTGCACCGATGTTCTCGC | Aeromonas hydrophila | Multiplex #1 |
| 39 | ACGCAGCAGTGCACCGATGT | Aeromonas hydrophila | Multiplex #1 |
| 40 | ACGCAGCAGTGCACCGAT | Aeromonas hydrophila | Multiplex #1 |
| 41 | GAAGAACGGGGTATGACGAC | Aeromonas hydrophila | Multiplex #1 |
| 42 | AGAACGGGGTATGACGAC | Aeromonas hydrophila | Multiplex #1 |
| 43 | GAACGGGGTATGACGAC | Aeromonas hydrophila | Multiplex #1 |
| 44 | ACAGAACCGCTTTTTGCAAG | Bacillus anthracis/Bacillus | Multiplex #1 |
| | | cereus | |
| 45 | TGAATTTAGCGTGAGCTTTT | Bacillus anthracis/Bacillus | Multiplex #1 |
| | | cereus | |
| 46 | AGATAATACGAAAACTTCAG | Bacillus anthracis/Bacillus | Multiplex #1 |
| | | cereus | |
| 47 | AGATAATACGAAAACTTC | Bacillus anthracis/Bacillus | Multiplex #1 |
| | | cereus | |
| 48 | TTGAATTTGCTGTGTGGAGT | Bacillus subtilis | Multiplex #1 |
| 49 | TGAATTTGCTGTGTGGAG | Bacillus subtilis | Multiplex #1 |
| 50 | TGCTTCACCACGGTCAAGGA | Capnocytophaga canimorsus | Multiplex #1 |
| 51 | CTTCACCACGGTCAAGGA | Capnocytophaga canimorsus | Multiplex #1 |
| 52 | TTGATTTCAGTTTTATCGAT | Capnocytophaga canimorsus | Multiplex #1 |
| 53 | TTCTTCACGCTTGATACCAC | Citrobacter braakii | Multiplex #1 |
| 54 | TTCTTCACGCTTGATACC | Citrobacter braakii | Multiplex #1 |
| 55 | TTCTTCACGCTTGATAC | Citrobacter braakii | Multiplex #1 |
| 56 | CGGCTTGATAGAGCCCGGCT | Citrobacter braakii/Klebsiella | Multiplex #1 |
| | | oxytoca | |
| 57 | CGGCTTGATAGAGCCCGG | Citrobacter braakii/Klebsiella | Multiplex #1 |
| | | oxytoca | |
| 58 | CGGCTTGATAGAGCCCG | Citrobacter braakii/Klebsiella | Multiplex #1 |
| | | oxytoca | |
| 59 | CGGCTTGATAGAGCCC | Citrobacter braakii/Klebsiella | Multiplex #1 |
| | | oxytoca | |
| 60 | CCCGGCTTAGCCAGTACC | Citrobacter freundii complex | Multiplex #1 |
| 61 | ATTGTTCCAACTTGAGCTAA | Clostridium perfringens | Multiplex #1 |
| 62 | ATTGTTCCAACTTGAGCT | Clostridium perfringens | Multiplex #1 |
| 63 | TGCGGGGTGTACTCGCCCGG | Corynebacterium jeikeium | Multiplex #1 |
| 64 | TGCGGGGTGTACTCGCCC | Corynebacterium jeikeium | Multiplex #1 |
| 65 | TGCGGGGTGTACTCGCC | Corynebacterium jeikeium | Multiplex #1 |
| 66 | TGCGGGGTGTACTCGC | Corynebacterium jeikeium | Multiplex #1 |
| 67 | GGCTTGATGCTGCCCGGCTT | Enterobacter aerogenes | Multiplex #1 |
| 68 | GGCTTGATGCTGCCCGGC | Enterobacter aerogenes | Multiplex #1 |
| 69 | GCCTGGCTTCGCCAGAAC | Enterobacter cloacae complex | Multiplex #1 |
| 70 | GGCTTGATTGAGCCTGGC | Enterobacter cloacae complex | Multiplex #1 |
| 71 | GGCTTGATTGAGCCTGG | Enterobacter cloacae | Multiplex #1 |
| 72 | GTTCTCGCCCGCACGGCCTT | Enterobacter sakazakii | Multiplex #1 |
| 73 | TCTCGCCCGCACGGCCTT | Enterobacter sakazakii | Multiplex #1 |
| 74 | TCTCGCCCGCACGGCCT | Enterobacter sakazakii | Multiplex #1 |
| 75 | TTCTCGCCCGCACGGC | Enterobacter sakazakii | Multiplex #1 |
| 76 | CACCTACGTTCTCGCCCGC | Enterobacter sakazakii | Multiplex #1 |
| 77 | CCTACGTTCTCGCCCGC | Enterobacter sakazakii | Multiplex #1 |
| 78 | GTGATTGTAGCTGGTTTAGC | Enterococcus faecalis | Multiplex #1 |
| 79 | GTGATTGTAGCTGGTTTA | Enterococcus faecalis | Multiplex #1 |
| 80 | TTTTGTGTGTGGAGTGATT | Enterococcus faecalis | Multiplex #1 |
| 81 | TACTTCAGCTTTGAATTTTG | Enterococcus faecalis | Multiplex #1 |
| 82 | GAGCGTAGTCTAACAATTT | Enterococcus faecium | Multiplex #1 |
| 83 | AGCGTAGTCTAACAATTT | Enterococcus faecium | Multiplex #1 |
| 84 | GTGTGATTGTACCTGGTTTA | Enterococcus faecium/ | Multiplex #1 |
| | | Enterococcus hirae | |
| 85 | TGTGATTGTACCTGGTT | Enterococcus faecium/ | Multiplex #1 |
| | | Enterococcus hirae | |
| 86 | TTCTTCTTTTGTCAACACGT | Enterococcus faecium/ | Multiplex #1 |
| | | Enterococcus hirae | |
| 87 | CTTCTTTTGTCAACACG | Enterococcus faecium/ | Multiplex #1 |
| | | Enterococcus hirae | |
| 88 | GCTTGATGGTGCCCGGCTTA | Escherichia coli, Escherichia | Multiplex #1 |
| | | fergusonii, Shigella sp., | |
| | | Salmonella choleraesuis | |
| 89 | CTTGATGGTGCCCGGCTT | Escherichia coli, Escherichia | Multiplex #1 |
| | | fergusonii, Shigella sp., | |
| | | Salmonella choleraesuis | |
| 90 | ACGTTCGATGTCTTCACGAG | Gemella haemolysans | Multiplex #1 |
| 91 | GTTCGATGTCTTCACGAG | Gemella haemolysans | Multiplex #1 |
| 92 | TTCGATGTCTTCACGAG | Gemella haemolysans | Multiplex #1 |
| 93 | ACATCAGCTACGAATTGAGT | Gemella morbillorum | Multiplex #1 |
| 94 | CATCAGCTACGAATTGAG | Gemella morbillorum | Multiplex #1 |
| 95 | ATCAGCTACGAATTGAG | Gemella morbillorum | Multiplex #1 |
| 96 | ACCGATGTTTTCACCTGCAC | Haemophilus influenzae | Multiplex #1 |
| 97 | CGATGTTTTCACCTGCA | Haemophilus influenzae | Multiplex #1 |
| 98 | CGATGTTTTCACCTGC | Haemophilus influenzae | Multiplex #1 |
| 99 | TTGAACCTGGTTTCGCTAAT | Haemophilus influenzae | Multiplex #1 |
| 100 | TGAACCTGGTTTCGCTAA | Haemophilus influenzae | Multiplex #1 |
| 101 | CACGCAACAATACACCAACG | Kingella kingae | Multiplex #1 |
| 102 | CACGCAATAATACACCAACG | Kingella kingae | Multiplex #1 |
| 103 | CTTCAGCTTCAAATTTAGTG | Kingella kingae | Multiplex #1 |
| 104 | TTCTTCTTTGCTCAACACAT | Kingella kingae | Multiplex #1 |
| 105 | TTCTTCTTTGCTCAATACAT | Kingella kingae | Multiplex #1 |
| 106 | TGCGGCTTGATAGAGCCC | Klebsiella oxytoca | Multiplex #1 |
| 107 | TTGGACAGGATATAAACTTC | Klebsiella oxytoca | Multiplex #1 |
| 108 | AGTGTGACGGCCGCCTTCGT | Klebsiella oxytoca | Multiplex #1 |
| 109 | CGGGTTGATGGTGCCCGGCT | Klebsiella pneumoniae | Multiplex #1 |
| 110 | GGGTTGATGGTGCCCGGC | Klebsiella pneumoniae | Multiplex #1 |
| 111 | GGTTGATGGTGCCCGGC | Klebsiella pneumoniae | Multiplex #1 |
| 112 | GTTGATGGTGCCCGGC | Klebsiella pneumoniae | Multiplex #1 |
| 113 | CAGAACACCGACGTTCTCAC | Morganella morganii | Multiplex #1 |
| 114 | GAACACCGACGTTCTCA | Morganella morganii | Multiplex #1 |
| 115 | TTCGATTTCTTCACGCTTGG | Morganella morganii | Multiplex #1 |
| 116 | CGATTTCTTCACGCTTGG | Morganella morganii | Multiplex #1 |
| 117 | GATTTCTTCACGCTTGG | Morganella morganii | Multiplex #1 |
| 118 | GTTGGCGAAAAACGGGGTAT | Neisseria gonorrhoeae | Multiplex #1 |
| 119 | TTGGCGAAAAACGGGGTA | Neisseria gonorrhoeae | Multiplex #1 |
| 120 | TCTTCTTTGCTCAGTACGTA | Neisseria meningitidis | Multiplex #1 |
| 121 | CTTCTTTGCTCAGTACGT | Neisseria meningitidis | Multiplex #1 |
| 122 | CGGTAGTTGGCGAAGAACGG | Neisseria meningitidis | Multiplex #1 |
| 123 | CGGTAGTTGGCGAAGAAC | Neisseria meningitidis | Multiplex #1 |
| 124 | GGTAGTTGGCGAAGAAC | Neisseria meningitidis | Multiplex #1 |
| 125 | TTTTGATAACACGTAAACTT | Pasteurella multocida | Multiplex #1 |
| 126 | CTGGTCGGCATAGGACGGAGC | Internal control tag sequence* | Multiplex #1 |
| | TTCGCGGTGGATGCCCCAG | | |
| 127 | GCATAGGACGGAGCTTCGCGG | Internal control tag sequence* | Multiplex #1 |
| | TGGATGCCC | | |
| 128 | GGACGGAGCTTCGCGGTGGA | Internal control tag sequence* | Multiplex #1 |
| 129 | GCGCCGCCGAACAGGCCTAC | Internal control tag sequence* | Multiplex #1 |
| | CTTGCCGCCCTTGGC | | |
| 130 | ATGATCCGGCCCAGGGTCGC | Internal control tag sequence | Multiplex #1 |
| 131 | CATGCCGCGAACGACATCCT | Propionibacterium acnes | Multiplex #1 |
| 132 | GGCTGTAGTGGGAGAAGAAC | Propionibacterium acnes | Multiplex #1 |
| 133 | ACCTACGTTCTCACCTGCAC | Proteus mirabilis | Multiplex #1 |
| 134 | TTCACGTTTTGTACCACGCA | Proteus mirabilis | Multiplex #1 |
| 135 | CACGTTTTGTACCACGCA | Proteus mirabilis | Multiplex #1 |
| 136 | CAGTACTTGTCCACGTTCGA | Proteus mirabilis | Multiplex #1 |
| 137 | CAAATTTGTTGTGTGGGTT | Proteus mirabilis | Multiplex #1 |
| 138 | CAAATTTGTTGTGTGGG | Proteus mirabilis | Multiplex #1 |
| 139 | AGCCTTTGAAGAATGGAG | Proteus mirabilis | Multiplex #1 |
| 140 | CTACGTTCTCACCTGCAC | Proteus mirabillis | Multiplex #1 |
| 141 | CTACGTTCTCACCTGCA | Proteus mirabillis | Multiplex #1 |
| 142 | ACCTGGTTTTGCCAGTACTT | Providencia rettgeri | Multiplex #1 |
| 143 | ACCTGGTTTTGCCAGTAC | Providencia rettgeri | Multiplex #1 |
| 144 | ACCTGGTTTTGCCAGTA | Providencia rettgeri | Multiplex #1 |
| 145 | GCAGCAGGATACCAACGTTC | Pseudomonas aeruginosa | Multiplex #1 |
| 146 | CAGCAGGATACCAACGT | Pseudomonas aeruginosa | Multiplex #1 |
| 147 | AGCAGGATACCAACGT | Pseudomonas aeruginosa | Multiplex #1 |
| 148 | GCCACGCTCTACGTCTTCAC | Pseudomonas aeruginosa | Multiplex #1 |
| 149 | GCCACGCTCTACGTCTTC | Pseudomonas aeruginosa | Multiplex #1 |
| 150 | GCCACGCTCTACGTCTT | Pseudomonas aeruginosa | Multiplex #1 |
| 151 | GCCACGCTCTACGTCT | Pseudomonas aeruginosa | Multiplex #1 |
| 152 | GGCTTGATGGTGCCCGGC | Salmonella choleraesuis | Multiplex #1 |
| 153 | GGCTTGATGGTGCCCGG | Salmonella choleraesuis | Multiplex #1 |
| 154 | GGCTTGATGGTGCCCG | Salmonella choleraesuis | Multiplex #1 |
| 155 | CTTTGCTCAGGATGTACAC | Serratia sp. | Multiplex #1 |
| 156 | CTTTGCTCAGGATGTACA | Serratia sp. | Multiplex #1 |
| 157 | CGATGTCTTCACGCTTGAT | Serratia liquefaciens | Multiplex #1 |
| 158 | CGATGTCTTCACGCTTGA | Serratia liquefaciens | Multiplex #1 |
| 159 | CACTTCTGAGTCGAACTTGG | Serratia liquefaciens | Multiplex #1 |
| 160 | CACTTCTGAGTCGAACTT | Serratia liquefaciens | Multiplex #1 |
| 161 | CAGATTCGAACTGGGTGTG | Serratia marcescens | Multiplex #1 |
| 162 | AGATTCGAACTGGGTGTG | Serratia marcescens | Multiplex #1 |
| 163 | CATCTTTGCTCAGGATGT | Serratia marcescens | Multiplex #1 |
| 164 | ATCTTTGCTCAGGATGT | Serratia marcescens | Multiplex #1 |
| 165 | TTCATCTTTGCTCAGGATGT | Serratia marcescens | Multiplex #1 |
| 166 | ATCTTTGCTCAGGATG | Serratia marcescens | Multiplex #1 |
| 167 | TGTGACGACCACCTTCATC | Serratia marcescens | Multiplex #1 |
| 168 | TGTGACGACCACCTTCAT | Serratia marcescens | Multiplex #1 |
| 169 | AACGTTGTCCCCTGCAAGAC | Streptococcus agalactiae | Multiplex #1 |
| 170 | AACGTTGTCCCCTGCAAG | Streptococcus agalactiae | Multiplex #1 |
| 171 | AACGTTGTCCCCTGCAA | Streptococcus agalactiae | Multiplex #1 |
| 172 | AACGTTGTCCCCTGCA | Streptococcus agalactiae | Multiplex #1 |
| 173 | AACACCACGAAGAAGAACAC | Streptococcus agalactiae | Multiplex #1 |
| 174 | TGGTTTAGCAAGAACTTGAC | Streptococcus agalactiae | Multiplex #1 |
| 175 | GTTTAGCAAGAACTTGA | Streptococcus agalactiae | Multiplex #1 |
| 176 | TAAACTTCACCTTTAAATTT | Streptococcus agalactiae | Multiplex #1 |
| 177 | GAAGAAGAACCCCTACGTTA | Streptococcus anginosus/ | Multiplex #1 |
| | | Streptococcus constellatus | |
| 178 | CAAGAACTTGTCCACGTTCG | Streptococcus anginosus/ | Multiplex #1 |
| | | Streptococcus constellatus | |
| 179 | CAAGAACTTGTCCACGTT | Streptococcus anginosus/ | Multiplex #1 |
| | | Streptococcus constellatus | |
| 180 | AAGAACACCAACGTTATCCC | Streptococcus bovis | Multiplex #1 |
| 181 | TCACGTTGGATACCACGA | Streptococcus bovis | Multiplex #1 |
| 182 | TCCACCTTCCTCTTTAGTAA | Streptococcus mutans | Multiplex #1 |
| 183 | ACCTTCCTCTTTAGTAA | Streptococcus mutans | Multiplex #1 |
| 184 | CTCCGGCAATACCTTCGTCA | Streptococcus salivarius | Multiplex #1 |
| 185 | CTCCGGCAATACCTTCG | Streptococcus salivarius | Multiplex #1 |
| 186 | AAGAACACCGACGTTATCTC | Streptococcus salivarius | Multiplex #1 |
| 187 | GAACCAGGTGCAGCCAATAC | Streptococcus salivarius | Multiplex #1 |
| 188 | AACCAGGTGCAGCCAATA | Streptococcus salivarius | Multiplex #1 |
| 189 | TACGTTGTCCCCTGCAAGAC | Streptococcus sanguinis | Multiplex #1 |
| 190 | TACGTTGTCCCCTGCAAG | Streptococcus sanguinis | Multiplex #1 |
| 191 | TACGTTGTCCCCTGCAA | Streptococcus sanguinis | Multiplex #1 |
| 192 | CTGGTTTAGAGATAACTTGA | Streptococcus suis | Multiplex #1 |
| 193 | GGTTTAGAGATAACTTGA | Streptococcus suis | Multiplex #1 |
| 194 | ACGTAGTAGGGCACCAACGT | Vibrio vulnificus | Multiplex #1 |
| 195 | ACGTAGTAGGGCACCAAC | Vibrio vulnificus | Multiplex #1 |
| 196 | ACGTAGTAGCGCACCAAC | Vibrio vulnificus | Multiplex #1 |
| 197 | TMGAACCTGGTTTAGCAAGA | Yersinia enterocolitica | Multiplex #1 |
| 198 | TAGAACCTGGTTTAGCAA | Yersinia enterocolitica | Multiplex #1 |
| 199 | TCGAACCTGGTTTAGCAA | Yersinia enterocolitica | Multiplex #1 |
| 200 | GGTTTGATAGAACCTGGTTT | Yersinia pestis/Yersinia | Multiplex #1 |
| | | pseudotuberculosis | |
| 201 | GGTTTGATAGAACCTGGT | Yersinia pestis/Yersinia | Multiplex #1 |
| | | pseudotuberculosis | |
| 202 | CACGCTGAACATCGTCACGC | Yersinia pestis/Yersinia | Multiplex #1 |
| | | pseudotuberculosis | |
| 203 | CGCTGAACATCGTCACG | Yersinia pestis/Yersinia | Multiplex #1 |
| | | pseudotuberculosis | |
| 204 | GACAGAAGTTCACGAACTT | Citrobacter complex | Multiplex #2 |
| 205 | ACAGAAGTTCACGAACTT | Citrobacter complex | Multiplex #2 |
| 206 | TTCCATTTCTACCAGTTCCA | Citrobacter freundii | Multiplex #2 |
| 207 | TCCATTTCTACCAGTTCC | Citrobacter freundii | Multiplex #2 |
| 208 | CCATTTCTACCAGTTCC | Citrobacter freundii | Multiplex #2 |
| 209 | AGTGTCGTCGCCCGGGAAAT | Citrobacter freundii | Multiplex #2 |
| 210 | TGTCGTCGCCCGGGAAAT | Citrobacter freundii | Multiplex #2 |
| 211 | GTCGTCGCCCGGGAAAT | Citrobacter freundii | Multiplex #2 |
| 212 | CACGAACGATCGGAGTGTCG | Citrobacter freundii | Multiplex #2 |
| 213 | GCAGTTCACGCACTTCCATC | Citrobacter koseri | Multiplex #2 |
| 214 | GCAGTTCACGCACTTCCA | Citrobacter koseri | Multiplex #2 |
| 215 | CGCACTTCCATCTCAACCA | Citrobacter koserii/ | Multiplex #2 |
| | | Enterobacter sakazakii | |
| 216 | CGAACTTCCATCTCAACC | Enterobacter aerogenes | Multiplex #2 |
| 217 | TGTGCTCACGAGTCTGAGGC | Enterobacter cloacae | Multiplex #2 |
| 218 | TGCTCACGAGTCTGAGGC | Enterobacter cloacae | Multiplex #2 |
| 219 | TGCTCACGAGTCTGAGG | Enterobacter cloacae | Multiplex #2 |
| 220 | TCTCTACCAGTTCCAGCAGC | Enterobacter cloacae | Multiplex #2 |
| 221 | TCTCTACCAGTTCCAGCA | Enterobacter cloacae | Multiplex #2 |
| 222 | CGTCGCCTGGGAAATCGTAC | Enterobacter cloacae | Multiplex #2 |
| 223 | GAACCACGAACGATTGG | Enterobacter cloacae complex | Multiplex #2 |
| 224 | GTCGTACTGAGACAGCAGCT | Enterobacter sakazakii | Multiplex #2 |
| 225 | AAGAATCCAGGAAGCCAG | Klebsiella oxytoca | Multiplex #2 |
| 226 | AGGTATCCAGGTGGCCAG | Klebsiella pneumoniae | Multiplex #2 |
| 227 | GTGGAGTAATCGAACCTGGT | Listeria monocytogenes | Multiplex #2 |
| 228 | TGGAGTAATCGAACCTGG | Listeria monocytogenes | Multiplex #2 |
| 229 | GGAGTAATCGAACCTGG | Listeria monocytogenes | Multiplex #2 |
| 230 | AAAACATAAGTTTCAGCTTT | Listeria monocytogenes | Multiplex #2 |
| 231 | ATTCGAAGTCAGTGTGTGGC | Pasteurella pneumotropica | Multiplex #2 |
| 232 | GCCACACACTGACTTCGAAT | Pasteurella pneumotropica | Multiplex #2 |
| 233 | TTCATCTTTTGATAATACGT | Pasteurella pneumotropica | Multiplex #2 |
| 234 | ACGTATTATCAAAAGATGAA | Pasteurella pneumotropica | Multiplex #2 |
| 235 | TGAAGAATGGCGTATGACGA | Pasteurella pneumotropica | Multiplex #2 |
| 236 | AAGAATGGCGTATGACGA | Pasteurella pneumotropica | Multiplex #2 |
| 237 | AGAATGGCGTATGACGA | Pasteurella pneumotropica | Multiplex #2 |
| 238 | GTGCGCACCTTCCAAGACCTG | Internal control tag sequence* | Multiplex #2 |
| | ATTCTCGCCCTGCAGAACT | | |
| 239 | ACCTTCCAAGACCTGATTCTCG | Internal control tag sequence* | Multiplex #2 |
| | CCCTGCAG | | |
| 240 | CCCCAACCGCCTGCAGCACTA | Internal control tag sequence* | Multiplex #2 |
| | CTACCAGTTTCAGG | | |
| 241 | TGTGCTCACGGGTCTGCGGC | Salmonella choleraesuis | Multiplex #2 |
| 242 | TAAGAATCCAGGAAGCCAG | Salmonella choleraesuis | Multiplex #2 |
| 243 | TAAGAATCCAGGAAGCCA | Salmonella choleraesuis | Multiplex #2 |
| 244 | CAGTATGTGGTGTAATTGAA | Staphylococcus aureus | Multiplex #2 |
| 245 | CAGTATGTGGTGTAATT | Staphylococcus aureus | Multiplex #2 |
| 246 | TCGTCTTTTGATAATACG | Staphylococcus aureus | Multiplex #2 |
| 247 | CGTCTTTTGATAATACG | Staphylococcus aureus | Multiplex #2 |
| 248 | TGGTGTAATAGAACCAGGAG | Staphylococcus epidermidis | Multiplex #2 |
| 249 | TGTAATAGAACCAGGAG | Staphylococcus epidermidis | Multiplex #2 |
| 250 | GGTGTAATAGAACCAGGA | Staphylococcus epidermidis | Multiplex #2 |
| 251 | GCGATAGTTAGTGAAGAATG | Staphylococcus epidermidis | Multiplex #2 |
| 252 | GCGATAGTTAGTGAAGAA | Staphylococcus epidermidis | Multiplex #2 |
| 253 | TTGTGTGAGGTGTGATTGAA | Staphylococcus haemolyticus | Multiplex #2 |
| 254 | TATACGTCTGCTTTAAATTTT | Staphylococcus haemolyticus | Multiplex #2 |
| 255 | CGTCTTTAGATAAAACGTAT | Staphylococcus haemolyticus | Multiplex #2 |
| 256 | TACGTCTGCTTTGAATTT | Staphylococcus hominis | Multiplex #2 |
| 257 | AAACATATACGTCTGCTTTG | Staphylococcus hominis | Multiplex #2 |
| 258 | AAACGTATACGTCTGCTTTG | Staphylococcus hominis | Multiplex #2 |
| 259 | CATCTTTTGATAAAACGTAT | Staphylococcus hominis | Multiplex #2 |
| 260 | CATCTTTTGATAAAACATAT | Staphylococcus hominis | Multiplex #2 |
| 261 | CTTCATCTTTTGATAAAACG | Staphylococcus hominis | Multiplex #2 |
| 262 | TTAGTGTGTGGTGTGATTGA | Staphylococcus | Multiplex #2 |
| | | saccharolyticus | |
| 263 | TAGTGTGTGGTGTGATTG | Staphylococcus | Multiplex #2 |
| | | saccharolyticus | |
| 264 | AAAACGTAAACTTCAGCTTT | Staphylococcus | Multiplex #2 |
| | | saccharolyticus | |
| 265 | CGTAAACATCCGCTTTGAAT | Staphylococcus saprophyticus | Multiplex #2 |
| 266 | CGTAAACATCCGCTTTGA | Staphylococcus saprophyticus | Multiplex #2 |
| 267 | GTGTAATTGAACCAGGAG | Staphylococcus warneri | Multiplex #2 |
| 268 | GTGTAATTGAACCAGGA | Staphylococcus warneri | Multiplex #2 |
| 269 | ATTTTGTATGTGGTGTAATT | Staphylococcus warneri | Multiplex #2 |
| 270 | CGTAAACTTCCGCTTTGAAT | Staphylococcus warneri | Multiplex #2 |
| 271 | GTAAACTTCCGCTTTGA | Staphylococcus warneri | Multiplex #2 |
| 272 | GTGACGTCCACCTTCGTC | Staphylococcus warneri | Multiplex #2 |
| 273 | GTGACGTCCACCTTCG | Staphylococcus warneri | Multiplex #2 |
| 274 | GCGCCTGAATCAATCAATTT | Streptococcus agalactiae | Multiplex #2 |
| 275 | TGCAATTTCAAGACCTTGTT | Streptococcus bovis | Multiplex #2 |
| 276 | GCACCAGAATCAATTAATTT | Streptococcus canis | Multiplex #2 |
| 277 | CCCCAAGCGCAGCAGCGTAA | Streptococcus dysgalactiae | Multiplex #2 |
| 278 | CCAAGCGCAGCAGCGTAA | Streptococcus dysgalactiae | Multiplex #2 |
| 279 | CAAGCGCAGCAGCGTAA | Streptococcus dysgalactiae | Multiplex #2 |
| 280 | AAGCGCAGCAGCGTAA | Streptococcus dysgalactiae | Multiplex #2 |
| 281 | AATTTCAAGTCCTTGTTCTC | Streptococcus dysgalactiae | Multiplex #2 |
| 282 | TTCAAGTCCTTGTTCTC | Streptococcus dysgalactiae | Multiplex #2 |
| 283 | AATCAATTTCCCAGCAATTT | Streptococcus gordonii | Multiplex #2 |
| 284 | AATCAATTTTCCTGCAATCT | Streptococcus mitis | Multiplex #2 |
| 285 | AATCAATTTTCCAGCAATTT | Streptococcus oralis | Multiplex #2 |
| 286 | GCAGCATAAGCTGGATCAAG | Streptococcus pneumoniae | Multiplex #2 |
| 287 | AATCAATTTTCCCGCAATCT | Streptococcus pneumoniae | Multiplex #2 |
| 288 | AACCAACATGGCTATCTCCG | Streptococcus pneumoniae | Multiplex #2 |
| 289 | CCCCAAGCGCAGCAGCATAA | Streptococcus pyogenes | Multiplex #2 |
| 290 | CCCCAAGCGCAGCAGCA | Streptococcus pyogenes | Multiplex #2 |
| 291 | ACAACCAGATCAACCGC | Streptococcus pyogenes | Multiplex #2 |
| 292 | CAACAACCAGATCAACCG | Streptococcus pyogenes | Multiplex #2 |
| 293 | GCACCTGAGTCAATCAGCTT | Streptococcus sanguinis | Multiplex #2 |
| 294 | AAGTCACGGTGACCGGGGGC | Aspergillus sp. | Multiplex #3 |
| 295 | TCACGGTGACCGGGGGC | Aspergillus sp. | Multiplex #3 |
| 296 | GCTCACGGGTCTGACCATC | Aspergillus flavus | Multiplex #3 |
| 297 | ATCGTGTTAGCTACAGCACC | Aspergillus fumigatus | Multiplex #3 |
| 298 | GATGAGCTGCTTGACACCGA | Aspergillus fumigatus | Multiplex #3 |
| 299 | ATGAGCTGCTTGACACCG | Aspergillus fumigatus | Multiplex #3 |
| 300 | GCAACAATGAGCTGACGGAC | Aspergillus nidulans | Multiplex #3 |
| 301 | CAACAATGAGCTGACGGA | Aspergillus nidulans | Multiplex #3 |
| 302 | ATGAGCTGGCGGACACCG | Aspergillus niger | Multiplex #3 |
| 303 | CAACGATGAGCTGGCGGA | Aspergillus niger | Multiplex #3 |
| 304 | GAGGGTGAAGGCAAGCAGAG | Aspergillus terreus | Multiplex #3 |
| 305 | AGGGTGAAGGCAAGCAGA | Aspergillus terreus | Multiplex #3 |
| 306 | GTTGGTGIATGGTTCAATCA | Candida albicans | Multiplex #3 |
| 307 | TTGGTGGATGGTTCAATC | Candida albicans | Multiplex #3 |
| 308 | TGGTGGATGGTTCAATC | Candida albicans | Multiplex #3 |
| 309 | ACCAGTAACTTTAICGGATT | Candida albicans | Multiplex #3 |
| 310 | CTTTACCGGATTTGGTTTCC | Candida albicans/Candida | Multiplex #3 |
| | | dublininensis | |
| 311 | CCTTACCGGATTTGGTTTCC | Candida albicans/Candida | Multiplex #3 |
| | | dublininensis | |
| 312 | TTACCGGATTTGGTTTCC | Candida albicans/Candida | Multiplex #3 |
| | | dublininensis | |
| 313 | GGTCTTACCAGTAACTTTAC | Candida albicans/Candida | Multiplex #3 |
| | | dublininensis | |
| 314 | GTCTTACCAGTAACTTTAC | Candida albicans/Candida | Multiplex #3 |
| | | dublininensis | |
| 315 | TGGTCTGGTTGGTGGTTC | Candida albicans/Candida | Multiplex #3 |
| | | dublininensis | |
| 316 | GTTGGTGGAAGCTICAATCA | Candida dubliniensis | Multiplex #3 |
| 317 | TTGGTGGAAGCTTCAATC | Candida dubliniensis | Multiplex #3 |
| 318 | CGATTTCAGCGAATCTGG | Candida glabrata | Multiplex #3 |
| 319 | TGTACCAGGAAGCGTTGGTG | Candida glabrata | Multiplex #3 |
| 320 | TACCAGGAAGCGTTGGTG | Candida glabrata | Multiplex #3 |
| 321 | GGTTGGTCTGACAGGTGG | Candida krusei | Multiplex #3 |
| 322 | TAATGGCTTTTCGGTTGG | Candida krusei | Multiplex #3 |
| 323 | TAATGGCTTTTCGGTTG | Candida krusei | Multiplex #3 |
| 324 | ATGGGACAGCTTTAGGGTTG | Candida parapsilosis | Multiplex #3 |
| 325 | ACCAGCTTTAGTTTCCTTTTCC | Candida parapsilosis | Multiplex #3 |
| 326 | CCTTACCAGCTTTAGTTTCC | Candida parapsilosis | Multiplex #3 |
| 327 | CCTTACCAGCTTTAGTTT | Candida parapsilosis | Multiplex #3 |
| 328 | CTTGGTTTCTTTTTCCCAAC | Candida tropicalis | Multiplex #3 |
| 329 | CTTGGTTTCTTTTTCCCA | Candida tropicalis | Multiplex #3 |
| 330 | CTTGGTTTCTTTTTCCC | Candida tropicalis | Multiplex #3 |
| 331 | TTGGTCTTGAAGGTGGTTCA | Candida tropicalis | Multiplex #3 |
| 332 | GGTCTTGAAGGTGGTTCA | Candida tropicalis | Multiplex #3 |
| 333 | GTCTTGAAGGTGGTTCA | Candida tropicalis | Multiplex #3 |
| 334 | TTGGGCGCTGCCGGCACCTGT | Internal control tag sequence* | Multiplex #3 |
| | CCTACGAGTTGCATGATAA | | |
| 335 | CTGCCGGCACCTGTCCTACGA | Internal control tag sequence* | Multiplex #3 |
| | GTTGCATGA | | |
| 336 | CCGGCACCTGTCCTACGAGT | Internal control tag sequence* | Multiplex #3 |
| 337 | GCGTGGGTATGGTGGCAGGC | Internal control tag sequence* | Multiplex #3 |
| 338 | CGGCAGCGGTGCGGACTGTT | Internal control tag sequence* | Multiplex #3 |
| | GTAACTCAGAATAAG | | |
| 339 | ATCGAAACTGGTGTTAT | Bacteroides fragilis | Multiplex #4 |
| 340 | CCTCGGTTTGGGTGAAG | Bacteroides fragilis | Multiplex #4 |
| 341 | AATCAGTTGTAACAGGT | Bacteroides fragilis | Multiplex #4 |
| 342 | CGTCGGCATCAAGGCGACGA | Brucella melitensis | Multiplex #4 |
| 343 | TCGGCATCAAGGCGACGA | Brucella melitensis | Multiplex #4 |
| 344 | CGGCATCAAGGCGACGA | Brucella melitensis | Multiplex #4 |
| 345 | CGAAGACCACGGTTACCGGC | Brucella melitensis | Multiplex #4 |
| 346 | AAGACCACGGTTACCGG | Brucella melitensis | Multiplex #4 |
| 347 | CGGCATCGTGAAGGTCGGCG | Burkholderia cepacia | Multiplex #4 |
| 348 | GGCATCGTGAAGGTCGG | Burkholderia cepacia | Multiplex #4 |
| 349 | AGCAGGAACGGCTTGTCA | Escherichia coli/Shigella sp. | Multiplex #4 |
| 350 | GAGAATACGTCTTCGATC | Escherichia coli/Shigella sp. | Multiplex #4 |
| 351 | ACTTCTTCACCAACTTTGAT | Escherichia coli/Shigella sp. | Multiplex #4 |
| 352 | CTTCTTCACCAACTTTGA | Escherichia coli/Shigella sp. | Multiplex #4 |
| 353 | GCGCCGCCCTATACCTTGTCT | Internal control tag sequence | Multiplex #4 |
| | GCCTCCCCGCGTTG | | |
| 354 | GACGACCATCAGGGACAGCTT | Internal control tag sequence | Multiplex #4 |
| | CAAGGATCGCTCGCGGCTC | | |
| 355 | ACCATCAGGGACAGCTTCAAG | Internal control tag sequence | Multiplex #4 |
| | GATCGCTCG | | |
| 356 | CCGTCCGGTGCAGAAGAC | Stenotrophomonas maltophilia | Multiplex #4 |
| 357 | CCGTCCGGTGCAGAAG | Stenotrophomonas maltophilia | Multiplex #4 |
| 358 | TCGTGGCACGGTCGTCA | Streptomyces avermitilis | Multiplex #4 |
| 359 | TCGTGGCACGGTCGTCACCGG | Streptomyces avermitilis | Multiplex #4 |
| | TCGT | | |
| 360 | TCGTGGCACGGTCGTCACCGG | Streptomyces avermitilis | Multiplex #4 |
| | TCGTATCGA | | |
| 361 | TGGCACGGTCGTCACCGGT | Streptomyces avermitilis | Multiplex #4 |
| 362 | CGTCGACATCGTCGGTATCA | Streptomyces avermitilis | Multiplex #4 |
| 363 | CGTCGACATCGTCGGTATCAA | Streptomyces avermitilis | Multiplex #4 |
| | GACCGAGAA | | |
| 364 | TATAGGTATCCAGGTGGCCAG | Klebsiella pneumoniae | Multiplex #2 |
| 365 | GGCCGAGGTTGATGCGATTGA | Internal control tag sequence* | Multiplex #2 |
| | CCACGGTGCCCTTG | | |
| 366 | GGCATCGTGAAGGTCG | Burkholderia cepacia | Multiplex #4 |
| 367 | TCAAGCCGACGGTGAAGAC | Burkholderia cepacia | Multiplex #4 |
| 368 | GAGCGTGCGATTGACAAGCCG | Escherichia coli/Shigella sp. | Multiplex #4 |
| | TTCC | | |
| 369 | TTCTCCATCTCCGGTCGTGGT | Escherichia coli/Shigella sp. | Multiplex #4 |
| | ACC | | |
| 370 | CATCAAAGTTGGTGAAGAAGTT | Escherichia coli/Shigella sp. | Multiplex #4 |
| | G | | |
| 371 | TCAAAGTTGGTGAAGAAG | Escherichia coli/Shigella sp. | Multiplex #4 |
| 372 | GAGCGCGGCGTGATCAAG | Stenotrophomonas maltophilia | Multiplex #4 |
| 373 | GGCGACGAAATCGAAATCG | Stenotrophomonas maltophilia | Multiplex #4 |
| 374 | GAAGACCACCGTGACCGG | Stenotrophomonas maltophilia | Multiplex #4 |

*The internal control template allows to verify the efficiency of each PCR amplification and/or microarray hybridization as well as to ensure that there is no significant inhibition of the nucleic acid amplification and/or detection processes. This internal control template may be preferably present in each PCR reaction.

TABLE 3
Number of designed and retained primers and probes for the present
invention.

| | Designed | Retained* |
|---|---|---|
| Primers - Bacteria | 85 | 19 |
| Primers - Fungi | 23 | 7 |
| Probes - Bacteria | 412 | 306 |
| Probes - Fungi | 90 | 45 |

*Primers and probes retained for the final multiplex combinations.

TABLE 4
List of the 73 tested bacterial and fungal species commonly associated
with bloodstream infection.

| | |
|---|---|
| Acinetobacter baumannii | Listeria monocytogenes |
| Acinetobacter lwoffii | Morganella morganii |
| Aeromonas caviae | Neisseria gonorrhoeae |
| Aeromonas hydrophila | Neisseria meningitidis |
| Aspergillus flavus | Pasteurella multocida |
| Aspergillus nidulans | Pasteurella pneumotropica |
| Aspergillus niger | Propionibacterium acnes |
| Aspergillus terreus | Proteus mirabillis |
| Bacillus anthracis/Bacillus cereus^{a} | Providencia rettgeri |
| Bacillus subtilis | Pseudomonas aeruginosa |
| Bacteroides fragilis | Salmonella choleraesuis |
| Brucella melitensis | Serratia liquefaciens |
| Burkholderia cepacia | Serratia marcescens |
| Candida albicans/Candida dubliniensis^{a} | Staphylococcus aureus |
| Candida glabrata | Staphylococcus epidermidis |
| Candida krusei | Staphylococcus haemolyticus |
| Candida parapsilosis | Staphylococcus hominis |
| Candida tropicalis | Staphylococcus saccharolyticus |
| Capnocytophaga canimorsus | Staphylococcus warneri |
| Citrobacter braakii | Stenotrophomonas maltophilia |
| Citrobacter freundii | Streptococcus agalactiae |
| Clostridium perfringens | Streptococcus anginosus |
| Corynebacterium jeikeium | Streptococcus bovis |
| Enterobacter aerogenes | Streptococcus constellatus |
| Enterobacter cloacae | Streptococcus dysgalactiae |
| Enterobacter sakazakii | Streptococcus mutans |
| Enterococcus faecalis | Streptococcus pneumoniae |
| Enterococcus faecium | Streptococcus pyogenes |
| Escherichia coli/Shigella sp. | Streptococcus salivarius |
| Gemella haemolysans | Streptococcus sanguinis |
| Gemella morbillorum | Streptococcus suis |
| Haemophilus influenzae | Vibrio vulnificus |
| Kingella kingae | Yersinia enterocolitica |
| Klebsiella oxytoca | Yersinia pestis/Yersinia |
| | pseudotuberculosis^{a} |
| Klebsiella pneumoniae | |

^{a}These phenotypic species are part of the same genetic species. Therefore, distinction of these phenotypic species using molecular probes may not be possible.

1.-57. (canceled)

58. A method of detecting a pathogen, the method comprising exposing a sample containing or suspected of containing a pathogen with oligonucleotide mixtures comprising multiple oligonucleotide species, wherein each oligonucleotide species is capable of specific binding with a genetic material of a pathogen selected from the group consisting of:
A) Acinetobacter baumannii, Acinetobacter Iwoffii, Aeromonas caviae, Aeromonas hydrophile, Bacillus cereus, Bacillus subtilis, Citrobacter braakii, Citrobacter freundii, Citrobacter koseri, Enterobacter aerogenes, Enterobacter cloacae, Enterobacter sakazakii, Enterococcus faecium, Gemella haemolysans, Gemella morbillorum, Haemophilus influenzae, Kingella kingae, Klebsiella oxytoca, Klebsiella pneumoniae, Morganella morganii, Neisseria gonorrhoeae, Neisseria meningitidis, Pasteurella multocida, Propionibacterium acnes, Proteus mirabilis, Providencia rettgeri, Pseudomonas aeruginosa, Salmonella choleraesuis, Serratia liquefaciens, Serratia marcescens, Streptococcus agalactiae, Streptococcus anginosus, Streptococcus bovis, Streptococcus mutans, Streptococcus salivarius, Streptococcus sanguinis, Streptococcus suis, Vibrio vulnificus, Yersinia enterocolitica, Yersinia pestis/Yersinia pseudotuberculosis, Enterococcus faecalis, Clostridium perfringens, Corynebacterium jeikeium, and Capnocytophaga canimorsus;
B) Citrobacter freundii, Citrobacter koseri, Enterobacter aerogenes, Enterobacter cloacae, Enterobacter sakazakii, Klebsiella oxytoca, Klebsiella pneumoniae, Salmonella choleraesuis, Listeria monocytogenes, Pasteurella pneumotropica, Staphylococcus aureus, Staphylococcus epidermidis, Staphylococcus haemolyticus, Staphylococcus hominis, Staphylococcus saccharolyticus, Staphylococccus saprophyticus, Staphylococcus warneri, Streptococcus dysgalactiae, Streptococcus pneumoniae, and Streptococcus pyogenes;
C) Candida albicans, Candida glabrata, Candida parapsilosis, Candida tropicalis, Candida krusei, Aspergillus fumigatus, Aspergillus niger, Aspergillus nidulans, Aspergillus flavus, and Aspergillus terreus;
D) Bacteroides fragilis, Brucella melitensis, Burkholderia cepacia, Stenotrophomonas maltophilia, Escherichia coli and Shigella sp; and
wherein for the pathogen of A), amplification is performed in the same vial or container with a combination of primers selected from the group consisting of:
a) a nucleic acid comprising from 0 to 5 nucleotide addition or deletion at a 5′ end of SEQ ID NO: 1,
b) a nucleic acid comprising from 0 to 5 nucleotide addition or deletion at a 5′ end of SEQ ID NO: 2,
c) a nucleic acid comprising from 0 to 5 nucleotide addition or deletion at a 5′ end of SEQ ID NO: 3,
d) a nucleic acid comprising from 0 to 5 nucleotide addition or deletion at a 5′ end of SEQ ID NO: 4,
e) a nucleic acid comprising from 0 to 5 nucleotide addition or deletion at a 5′ end of SEQ ID NO: 5,
f) a nucleic acid comprising from 0 to 5 nucleotide addition or deletion at a 5′ end of SEQ ID NO: 6,
g) a nucleic acid comprising from 0 to 5 nucleotide addition or deletion at a 5′ end of SEQ ID NO: 7,
h) a nucleic acid comprising from 0 to 5 nucleotide addition or deletion at a 5′ end of SEQ ID NO: 8,
i) a nucleic acid comprising from 0 to 5 nucleotide addition or deletion at a 5′ end of SEQ ID NO: 375, and
j) a nucleic acid comprising from 0 to 5 nucleotide addition or deletion at a 5′ end of SEQ ID NO: 376;
wherein for the pathogen of B), amplification is performed in the same vial or container with a combination of primers selected from the group consisting of:
k) a nucleic acid comprising from 0 to 5 nucleotide addition or deletion at a 5′ end of SEQ ID NO: 9,
l) a nucleic acid comprising from 0 to 5 nucleotide addition or deletion at a 5′ end of SEQ ID NO: 10,
m) a nucleic acid comprising from 0 to 5 nucleotide addition or deletion at a 5′ end of SEQ ID NO: 11,
n) a nucleic acid comprising from 0 to 5 nucleotide addition or deletion at a 5′ end of SEQ ID NO: 12,
o) a nucleic acid comprising from 0 to 5 nucleotide addition or deletion at a 5′ end of SEQ ID NO: 13, and
p) a nucleic acid comprising from 0 to 5 nucleotide addition or deletion at a 5′ end of SEQ ID NO: 14;
wherein for the pathogen of C), amplification is performed in the same vial or container with a combination of primers selected from the group consisting of:
q) a nucleic acid comprising from 0 to 5 nucleotide addition or deletion at a 5′ end of SEQ ID NO: 15,
r) a nucleic acid comprising from 0 to 5 nucleotide addition or deletion at a 5′ end of SEQ ID NO: 16,
s) a nucleic acid comprising from 0 to 5 nucleotide addition or deletion at a 5′ end of SEQ ID NO: 17,
t) a nucleic acid comprising from 0 to 5 nucleotide addition or deletion at a 5′ end of SEQ ID NO: 18,
u) a nucleic acid comprising from 0 to 5 nucleotide addition or deletion at a 5′ end of SEQ ID NO: 19,
v) a nucleic acid comprising from 0 to 5 nucleotide addition or deletion at a 5′ end of SEQ ID NO: 20, and
w) a nucleic acid comprising from 0 to 5 nucleotide addition or deletion at a 5′ end of SEQ ID NO: 21;
wherein for the pathogen of D), amplification is performed in the same vial or container with a combination of primers selected from the group consisting of:
x) a nucleic acid comprising from 0 to 5 nucleotide addition or deletion at a 5′ end of SEQ ID NO: 22,
y) a nucleic acid comprising from 0 to 5 nucleotide addition or deletion at a 5′ end of SEQ ID NO: 23,
z) a nucleic acid comprising from 0 to 5 nucleotide addition or deletion at a 5′ end of SEQ ID NO: 24,
aa) a nucleic acid comprising from 0 to 5 nucleotide addition or deletion at a 5′ end of SEQ ID NO: 25,
bb) a nucleic acid comprising from 0 to 5 nucleotide addition or deletion at a 5′ end of SEQ ID NO: 26,
cc) a nucleic acid comprising from 0 to 5 nucleotide addition or deletion at a 5′ end of SEQ ID NO: 377, and
dd) a nucleic acid comprising from 0 to 5 nucleotide addition or deletion at a 5′ end of SEQ ID NO: 378.

59. The method of claim 58, wherein the multiple oligonucleotide species comprise multiple sets of primer pairs capable of specific amplification of the genetic material and wherein the sample is exposed with the multiple sets of primer pairs under conditions suitable for nucleic acid amplification.

60. The method of claim 58, wherein the multiple oligonucleotide species comprises probes, each probe being capable of hybridizing with the genetic material of one or more pathogen species and wherein the sample is exposed with the probe under conditions suitable for hybridization.

61. The method of claim 59, wherein the probe is selected from the group consisting of a nucleic acid comprising from 0 to 5 nucleotide addition, deletion or combination of addition and deletion at a 5′ end and/or 3′ end thereof of any one of SEQ ID NO: 27 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373, or SEQ ID NO: 374, complement and combination thereof.

62. The method of claim 58, wherein the amplification of the genetic material of each pathogen is performed simultaneously.

63. The method of claim 58, wherein the genetic material is RNA or DNA.

64. An oligonucleotide selected from the group consisting of:
a) an oligonucleotide comprising or consisting of the sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8;
b) an oligonucleotide comprising or consisting of the sequence selected from the group consisting of SEQ ID NO: 9, SEQ ID NO: 10, SEQ ID NO: 11, SEQ ID NO: 12, SEQ ID NO: 13 and SEQ ID NO: 14;
c) an oligonucleotide comprising or consisting of the sequence selected from the group consisting of SEQ ID NO: 15, SEQ ID NO: 16, SEQ ID NO: 17, SEQ ID NO: 18, SEQ ID NO: 19, SEQ ID NO: 20 and SEQ ID NO: 21;
d) an oligonucleotide comprising or consisting of the sequence selected from the group consisting of SEQ ID NO: 22, SEQ ID NO: 23, SEQ ID NO: 24, SEQ ID NO: 25 and SEQ ID NO: 26;
e) the oligonucleotide of any one of a) to d) comprising from 0 to 5 additional nucleotides at a 5′ end thereof;
f) the oligonucleotide of any one of a) to d) comprising from 0 to 5 nucleotides deletion at a 5′ end thereof; and
g) a complement of any one of the above.

65. The oligonucleotide of claim 60, wherein said oligonucleotide comprises a label.

66. A kit for detecting a pathogen comprising an oligonucleotide according to claim 64.

67. A mixture, combination or composition of oligonucleotides for detecting a pathogen, comprising:
A) SEQ ID NO: 375, SEQ ID NO: 376 or combination thereof and an oligonucleotide selected from the group consisting of:
a) a nucleic acid comprising from 0 to 5 nucleotide addition or deletion at a 5′ end of SEQ ID NO: 1,
b) a nucleic acid comprising from 0 to 5 nucleotide addition or deletion at a 5′ end of SEQ ID NO: 2,
c) a nucleic acid comprising from 0 to 5 nucleotide addition or deletion at a 5′ end of SEQ ID NO: 3,
d) a nucleic acid comprising from 0 to 5 nucleotide addition or deletion at a 5′ end of SEQ ID NO: 4,
e) a nucleic acid comprising from 0 to 5 nucleotide addition or deletion at a 5′ end of SEQ ID NO: 5,
f) a nucleic acid comprising from 0 to 5 nucleotide addition or deletion at a 5′ end of SEQ ID NO: 6,
g) a nucleic acid comprising from 0 to 5 nucleotide addition or deletion at a 5′ end of SEQ ID NO: 7,
h) a nucleic acid comprising from 0 to 5 nucleotide addition or deletion at a 5′ end of SEQ ID NO: 8,
i) a complement of any one of a) to h), and
j) combination of any one of a) to h);
B)
k) a nucleic acid comprising from 0 to 5 nucleotide addition or deletion at a 5′ end of SEQ ID NO: 9,
l) a nucleic acid comprising from 0 to 5 nucleotide addition or deletion at a 5′ end of SEQ ID NO: 10,
m) a nucleic acid comprising from 0 to 5 nucleotide addition or deletion at a 5′ end of SEQ ID NO: 11,
n) a nucleic acid comprising from 0 to 5 nucleotide addition or deletion at a 5′ end of SEQ ID NO: 12,
o) a nucleic acid comprising from 0 to 5 nucleotide addition or deletion at a 5′ end of SEQ ID NO: 13,
p) a nucleic acid comprising from 0 to 5 nucleotide addition or deletion at a 5′ end of SEQ ID NO: 14,
q) a complement of any one of k) to p), and;
r) combination of any one of k) to p);
C)
s) a nucleic acid comprising from 0 to 5 nucleotide addition or deletion at a 5′ end of SEQ ID NO: 15,
t) a nucleic acid comprising from 0 to 5 nucleotide addition or deletion at a 5′ end of SEQ ID NO: 16,
u) a nucleic acid comprising from 0 to 5 nucleotide addition or deletion at a 5′ end of SEQ ID NO: 17,
v) a nucleic acid comprising from 0 to 5 nucleotide addition or deletion at a 5′ end of SEQ ID NO: 18,
w) a nucleic acid comprising from 0 to 5 nucleotide addition or deletion at a 5′ end of SEQ ID NO: 19,
x) a nucleic acid comprising from 0 to 5 nucleotide addition or deletion at a 5′ end of SEQ ID NO: 20,
y) a nucleic acid comprising from 0 to 5 nucleotide addition or deletion at a 5′ end of SEQ ID NO: 21,
z) a complement of any one of s) to y), and
aa) combination of any one of s) to y);
D) SEQ ID NO: 377, SEQ ID NO: 378 and combination thereof and an oligonucleotide selected from the group consisting of:
bb) a nucleic acid comprising from 0 to 5 nucleotide addition or deletion at a 5′ end of SEQ ID NO: 22,
cc) a nucleic acid comprising from 0 to 5 nucleotide addition or deletion at a 5′ end of SEQ ID NO: 23,
dd) a nucleic acid comprising from 0 to 5 nucleotide addition or deletion at a 5′ end of SEQ ID NO: 24,
ee) a nucleic acid comprising from 0 to 5 nucleotide addition or deletion at a 5′ end of SEQ ID NO: 25,
ff) a complement of any one of bb) to ee), and
gg) combination of any one of bb) to ee);
E)
hh) a nucleic acid comprising from 0 to 5 nucleotide addition or deletion at a 5′ end of SEQ ID NO: 22,
ii) a nucleic acid comprising from 0 to 5 nucleotide addition or deletion at a 5′ end of SEQ ID NO: 23,
jj) a nucleic acid comprising from 0 to 5 nucleotide addition or deletion at a 5′ end of SEQ ID NO: 26,
kk) a complement of any one of hh) to jj), and
ll) combination of any one of hh) to jj).

68. An oligonucleotide for detecting a pathogen, wherein said oligonucleotide is selected from the group consisting of:
a) an oligonucleotide having or consisting of the sequence selected from the group consisting of any one of SEQ ID NO: 27 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373, or SEQ ID NO: 374;
b) the nucleic acid of a) comprising from 0 to 5 additional nucleotides at a 5′ end and/or 3′ end thereof,
c) the nucleic acid of a) comprising from 0 to 5 nucleotides deletion at a 5′ end and/or 3′ end thereof,
d) a nucleic acid of a) comprising from 0 to 5 additional nucleotides at one of a 5′ end or 3′ end and a deletion of from 0 to 5 nucleotides at the other of a 5′ end or 3′ end thereof, and;
e) a complement of any one of the above.

69. The oligonucleotide of claim 68, wherein said oligonucleotide is selected from the group consisting of:
a) an oligonucleotide having or consisting of the sequence selected from the group consisting of SEQ ID NO: 27 to SEQ ID NO: 125, SEQ ID NO: 131 to SEQ ID NO. 202 or SEQ ID NO: 203;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

70. The oligonucleotide of claim 68, wherein said oligonucleotide is selected from the group consisting of:
a) an oligonucleotide having or consisting of the sequence selected from the group consisting of SEQ ID NO: 204 to SEQ ID NO: 237, SEQ ID NO: 241 to SEQ ID NO. 293 or SEQ ID NO: 364;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

71. The oligonucleotide of claim 68, wherein said oligonucleotide is selected from the group consisting of:
a) an oligonucleotide having or consisting of the sequence selected from the group consisting of SEQ ID NO: 294 to SEQ ID NO: 332 or SEQ ID NO: 333;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

72. The oligonucleotide of claim 68, wherein said oligonucleotide is selected from the group consisting of:
a) an oligonucleotide having or consisting of the sequence selected from the group consisting of SEQ ID NO: 339 to SEQ ID NO: 352, SEQ ID NO: 356, SEQ ID NO: 357, SEQ ID NO: 366 to SEQ ID NO: 373 or SEQ ID NO: 374;
b) the oligonucleotide of a) wherein the oligonucleotide comprises from 0 to 5 additional nucleotides at a 5′ end and/or 3′ end thereof,
c) the oligonucleotide of a) wherein the oligonucleotide comprises a deletion of from 0 to 5 nucleotides at a 5′ end and/or 3′ end thereof,
d) the oligonucleotide of a) wherein the oligonucleotide comprises from 0 to 5 additional nucleotides at one of a 5′ end or 3′ end and a deletion of from 0 to 5 nucleotides at the other of a 5′ end or 3′ end thereof, and;
e) a complement of any one of the above.

73. A solid support comprising a plurality of oligonucleotides attached thereto, wherein each oligonucleotide comprises a different nucleic acid sequence and is capable of specific binding to a pathogen selected from the group consisting of:

| | |
|---|---|
| Acinetobacter baumannii, | Klebsiella pneumoniae, |
| Acinetobacter lwoffii, | Listeria monocytogenes, |
| Aeromonas caviae, | Morganella morganii, |
| Aeromonas hydrophila, | Neisseria gonorrhoeae, |
| Aspergillus flavus, | Neisseria meningitidis, |
| Aspergillus nidulans, | Pasteurella multocida, |
| Aspergillus niger, | Pasteurella pneumotropica, |
| Aspergillus terreus, | Propionibacterium acnes, |
| Bacillus anthracis, | Proteus mirabillis, |
| Bacillus cereus, | Providencia rettgeri, |
| Bacillus subtilis, | Pseudomonas aeruginosa, |
| Bacteroides fragilis, | Salmonella choleraesuis, |
| Brucella melitensis, | Serratia liquefaciens, |
| Burkholderia cepacia, | Serratia marcescens, |
| Candida albicans, | Staphylococcus aureus, |
| Candida dubliniensis, | Staphylococcus epidermidis, |
| Candida glabrata, | Staphylococcus haemolyticus, |
| Candida krusei, | Staphylococcus hominis, |
| Candida parapsilosis, | Staphylococcus saccharolyticus, |
| Candida tropicalis, | Staphylococcus warneri, |
| Capnocytophaga canimorsus, | Stenotrophomonas maltophilia, |
| Citrobacter braakii, | Streptococcus agalactiae, |
| Citrobacter freundii, | Streptococcus anginosus, |
| Clostridium perfringens, | Streptococcus bovis, |
| Corynebacterium jeikeium, | Streptococcus constellatus, |
| Enterobacter aerogenes, | Streptococcus dysgalactiae, |
| Enterobacter cloacae, | Streptococcus mutans, |
| Enterobacter sakazakii, | Streptococcus pneumoniae, |
| Enterococcus faecalis, | Streptococcus pyogenes, |
| Enterococcus faecium, | Streptococcus salivarius, |
| Escherichia coli, | Streptococcus sanguinis, |
| Shigella sp., | Streptococcus suis, |
| Gemella haemolysans, | Vibrio vulnificus, |
| Gemella morbillorum, | Yersinia enterocolitica, |
| Haemophilus influenzae, | Yersinia pestis, |
| Kingella kingae, | Yersinia pseudotuberculosis and; |
| Klebsiella oxytoca, | |

each of said oligonucleotide independently comprising from 10 to 50 nucleotides.

74. The solid support of claim 73, wherein said plurality of oligonucleotides is selected from the group consisting of:
a) an oligonucleotide having or consisting of the sequence selected from the group consisting of SEQ ID NO: 27 to SEQ ID NO: 44, SEQ ID NO: 46 to SEQ ID NO: 63, SEQ ID NO: 65 to SEQ ID NO: 71, SEQ ID NO: 73 to SEQ ID NO: 77, SEQ ID NO: 79 to SEQ ID NO: 97, SEQ ID NO: 99 to SEQ ID NO: 125, SEQ ID NO: 131 to SEQ ID NO. 202 and SEQ ID NO: 203;
b) an oligonucleotide having or consisting of the sequence selected from the group consisting of SEQ ID NO: 204, SEQ ID NO: 208, SEQ ID NO: 211, SEQ ID NO: 212, SEQ ID NO: 214, SEQ ID NO: 215, SEQ ID NO: 219, SEQ ID NO: 223, SEQ ID NO: 226, SEQ ID NO: 227, SEQ ID NO: 229, SEQ ID NO: 231, SEQ ID NO: 233, SEQ ID NO: 236, SEQ ID NO: 241, SEQ ID NO: 242, SEQ ID NO: 244, SEQ ID NO: 246, SEQ ID NO: 248, SEQ ID NO: 249, SEQ ID NO: 253 to SEQ ID NO: 256, SEQ ID NO: 261, SEQ ID NO: 264 to SEQ ID NO: 267, SEQ ID NO: 270, SEQ ID NO: 272, SEQ ID NO: 279 to SEQ ID NO: 281, SEQ ID NO: 284 to SEQ ID NO: 288, SEQ ID NO: 291, SEQ ID NO: 292 and SEQ ID NO: 364;
c) an oligonucleotide having or consisting of the sequence selected from the group consisting of SEQ ID NO: 294, SEQ ID NO: 296 to SEQ ID NO:309, SEQ ID NO: 312, SEQ ID NO: 314, SEQ ID NO: 316, SEQ ID NO:317, SEQ ID NO: 318, SEQ ID NO: 320 to SEQ ID NO: 323, SEQ ID NO:326 to SEQ ID NO: 330 and SEQ ID NO: 332;
d) an oligonucleotide having or consisting of the sequence selected from the group consisting of SEQ ID NO: 339 to SEQ ID NO: 344, SEQ ID NO:348, SEQ ID NO: 366 to SEQ ID NO: 373 and SEQ ID NO: 374;
e) the oligonucleotide of any one of a) to d), wherein the oligonucleotide comprises from 0 to 5 additional nucleotides at a 5′ end and/or 3′ end thereof,
f) the oligonucleotide of any one of a) to d), wherein the oligonucleotide comprises a deletion of from 0 to 5 nucleotides at a 5′ end and/or 3′ end thereof,
g) the oligonucleotide of any one of a) to d), wherein the oligonucleotide comprises from 0 to 5 additional nucleotides at one of a 5′ end or 3′ end and a deletion of from 0 to 5 nucleotides at the other of a 5′ end or 3′ end thereof, and
h) a complement of any one of a) to g).

75. A method for the diagnosis of a bloodstream infection in an individual in need, the method comprising detecting the presence or absence of a pathogen from a sample obtained from the individual with oligonucleotides capable of specific binding with genetic material of a pathogen selected from the group consisting of:

| | |
|---|---|
| Acinetobacter baumannii, | Klebsiella pneumoniae, |
| Acinetobacter lwoffii, | Listeria monocytogenes, |
| Aeromonas caviae, | Morganella morganii, |
| Aeromonas hydrophila, | Neisseria gonorrhoeae, |
| Aspergillus flavus, | Neisseria meningitidis, |
| Aspergillus nidulans, | Pasteurella multocida, |
| Aspergillus niger, | Pasteurella pneumotropica, |
| Aspergillus terreus, | Propionibacterium acnes, |
| Bacillus anthracis, | Proteus mirabillis, |
| Bacillus cereus, | Providencia rettgeri, |
| Bacillus subtilis, | Pseudomonas aeruginosa, |
| Bacteroides fragilis, | Salmonella choleraesuis, |
| Brucella melitensis, | Serratia liquefaciens, |
| Burkholderia cepacia, | Serratia marcescens, |
| Candida albicans, | Staphylococcus aureus, |
| Candida dubliniensis, | Staphylococcus epidermidis, |
| Candida glabrata, | Staphylococcus haemolyticus, |
| Candida krusei, | Staphylococcus hominis, |
| Candida parapsilosis, | Staphylococcus saccharolyticus, |
| Candida tropicalis, | Staphylococcus warneri, |
| Capnocytophaga canimorsus, | Stenotrophomonas maltophilia, |
| Citrobacter braakii, | Streptococcus agalactiae, |
| Citrobacter freundii, | Streptococcus anginosus, |
| Clostridium perfringens, | Streptococcus bovis, |
| Corynebacterium jeikeium, | Streptococcus constellatus, |
| Enterobacter aerogenes, | Streptococcus dysgalactiae, |
| Enterobacter cloacae, | Streptococcus mutans, |
| Enterobacter sakazakii, | Streptococcus pneumoniae, |
| Enterococcus faecalis, | Streptococcus pyogenes, |
| Enterococcus faecium, | Streptococcus salivarius, |
| Escherichia coli, | Streptococcus sanguinis, |
| Shigella sp., | Streptococcus suis, |
| Gemella haemolysans, | Vibrio vulnificus, |
| Gemella morbillorum, | Yersinia enterocolitica, |
| Haemophilus influenzae, | Yersinia pestis, |
| Kingella kingae, | Yersinia pseudotuberculosis and; |
| Klebsiella oxytoca, | |

wherein the genetic material is detected with an oligonucleotide selected from the group consisting of any one of SEQ ID NO: 1 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373 and SEQ ID NO: 374, and wherein the presence of the pathogen is indicative of a bloodstream infection associated with the pathogen detected.

76. The method of claim 75, wherein the genetic material is detected with any one or all of SEQ ID NO: 375, SEQ ID NO: 376, SEQ ID NO: 377 or SEQ ID NO: 378 and with an oligonucleotide selected from the group consisting of any one of SEQ ID NO: 1 to SEQ ID NO: 125, SEQ ID NO: 131 to SEQ ID NO: 237, SEQ ID NO: 241 to SEQ ID NO: 333, SEQ ID NO: 339 to SEQ ID NO: 352, SEQ ID NO: 356, SEQ ID NO: 357, SEQ ID NO: 364, SEQ ID NO: 366 to SEQ ID NO: 373 and SEQ ID NO: 374.

77. The method of claim 75, wherein the genetic material is detected with SEQ ID NO: 26 and/or with SEQ ID NO: 378.
